(19) United States
(12) Patent Application Publication
(10) Pub. No.: US 2008/0139628 A1
(43) Pub. Date: Jun. 12, 2008

(54) Compositions For Treating Flushing And Lipid-Associated Disorders Comprising Niacin Receptor Partial Agonists

(75) Inventors: Dominic P. Behan, San Diego, CA (US); Daniel T. Connolly, Solana Beach, CA (US); Jeremy G. Richman, San Diego, CA (US)

Correspondence Address: FISH & RICHARDSON P.C., P.O. BOX 1022, MINNEAPOLIS, MN, 55440-1022, US

(73) Assignee: ARENA PHARMACEUTICALS, INC., San Diego, CA (US)

(21) Appl. No.: 11/718,539

(22) PCT Filed: Nov. 1, 2005
(86) PCT No.: PCT/US05/39560

§ 371 Date: Oct. 4, 2007

Related U.S. Application Data
(60) Provisional application No. 60/625,536, filed on Nov. 5, 2004.

(51) Int. Cl. A61K 31/4406; C07D 403/04; A61K 31/41; A61P 3/00; C07D 231/14; A61K 31/415

(52) U.S. Cl. 514/356; 548/254; 514/381; 548/374.1; 514/406

(57) ABSTRACT

The invention provides a method of reducing flushing induced by niacin or a niacin analog in a subject, comprising administering to said subject an effective flush reducing amount of a niacin receptor partial agonist. In addition, the invention provides a method of reducing flushing induced by niacin or a niacin analog in a subject, comprising administering to said subject an effective flush reducing amount of a niacin receptor partial agonist and an effective lipid altering amount of niacin or a niacin analog. The invention further provides a method of reducing flushing induced by niacin or a niacin analog in a subject, comprising administering to said subject an effective flush reducing amount of a niacin receptor partial agonist and subsequently administering to said subject an effective lipid altering amount of niacin or a niacin analog.

### FIELD OF THE INVENTION

The present invention relates generally to treatment of lipid-associated disorders such as atherosclerosis and, more specifically, to compositions and methods for prevention of flushing induced by niacin therapy.

### BACKGROUND OF THE INVENTION

Atherosclerosis is a process where deposits of fatty substances, cholesterol and other substances build up in the inner lining of an artery. This buildup is called plaque. Plaques that rupture cause blood clots to form that can block blood flow to the heart (heart attack) or the brain (stroke). Heart attack is the number one cause of death for both men and women in the United States and stroke is the number three cause of death [see, for example, Nature Medicine, Special Focus on Atherosclerosis, (2002) 8:1209-1262]. Abnormally high levels of circulating lipids are a major predisposing factor in development of atherosclerosis. Elevated levels of low density lipoprotein (LDL) cholesterol, elevated levels of triglycerides, or low levels of high density lipoprotein (HDL) cholesterol are, independently, risk factors for atherosclerosis and associated pathologies.

Niacin (nicotinic acid, pyridine-3-carboxylic acid, vitamin B3) is a water-soluble vitamin required by the human body for health, growth and reproduction. Niacin is also one of the oldest used drugs for the treatment of lipid-associated disorders. It is a valuable drug in that it favorably affects virtually all of the lipid parameters listed above [Goodman and Gilman's Pharmacological Basis of Therapeutics, editors Harmon J G and Limbird L E, Chapter 36, Mahley R W and Bersot T P (2001) pages 971-1002]. The benefits of niacin in the treatment or prevention of atherosclerotic cardiovascular disease have been documented in six major clinical trials [Guyton J R (1998) Am J Cardiol 82:18U-23U]. Structure and synthesis of analogs or derivatives of niacin are discussed throughout the Merck Index, An Encyclopedia of Chemicals, Drugs, and Biologicals, Tenth Edition (1983).

Unfortunately, the doses of niacin required to alter serum lipid levels can be quite large and at these dosages adverse side effects are frequent. Side effects can include gastrointestinal disturbances, liver toxicity, and disruption of glucose metabolism and uric acid levels. However, the most frequent and prominent side effect of niacin therapy is intense flushing, often accompanied by cutaneous itching, tingling and warmth. Although the flushing reaction is generally harmless, it is sufficiently unpleasant that patient compliance is markedly reduced. Often, 30-40% of patients cease taking niacin treatment within days after initiating therapy.

Efforts have been undertaken to develop niacin analogs, dosage forms and treatment protocols which minimize the cutaneous flush reaction while maintaining therapeutic efficacy. However, to date, these efforts have resulted in compounds or methods that only partially reduce the cutaneous flush reaction. In addition, these compounds or methods can result in other side effects. For example, compounds such as aspirin can be administered before administering niacin in an attempt to reduce flushing. However, at best, aspirin only results in a partial reduction of flushing in some patients, and the gastrointestinal side effects of aspirin limit its use. In addition, extended or sustained release formulations of niacin have been developed that reportedly have a lower incidence of flushing. However, these extended or sustained release formulations have been shown to result in liver toxicity which is a more severe side effect than flushing.

Thus, there exists a need for compounds and methods that safely reduce or eliminate flushing induced by niacin or a niacin analog. The present invention satisfies this need and provides related advantages as well.

### SUMMARY OF THE INVENTION

The invention provides a method of reducing flushing induced by niacin or a niacin analog in a subject, comprising administering to said subject an effective flush reducing amount of a niacin receptor partial agonist. In one embodiment, said flushing is induced by niacin and in another embodiment, said flushing is induced by a niacin analog. In one embodiment, said niacin analog is a structural analog of niacin and in another embodiment, said niacin analog is a functional analog of niacin. In one embodiment, said niacin receptor partial agonist comprises a compound of Formula (I):

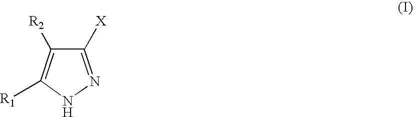

or a pharmaceutically acceptable salt thereof, wherein: X is a carboxyl or a tetrazol-5-yl group; R₁ is iso-propyl, 3-fluoro-benzyl, 3-chloro-benzyl, or 3-bromo-benzyl; and R₂ is H; or R₁ and R₂ together with the two pyrazole ring carbons to which they are bonded form a 5-membered carbocyclic ring optionally substituted with ethyl or a 5-membered heterocyclic ring optionally substituted with methyl. For example, said niacin receptor partial agonist can comprise a compound selected from the group consisting of: 3-(1H-Tetrazol-5-yl)-1,4,5,6-tetrahydro-cyclopentapyrazole; 5-(3-Fluoro-benzyl)-1H-pyrazole-3-carboxylic acid; 5-(3-Chloro-benzyl)-1H-pyrazole-3-carboxylic acid; 5-(3-Bromo-benzyl)-1H-pyrazole-3-carboxylic acid; 6-Methyl-3-(1H-tetrazol-5-yl)-4,6-dihydro-1H-furo[3,4-c]pyrazole; 3-(1H-Tetrazol-5-yl)-4,6-dihydro-1H-thieno[3,4-c]pyrazole; 3-(1H-Tetrazol-5-yl)-1,4-dihydro-cyclopentapyrazole; 3-(1H-Tetrazol-5-yl)-1,6-dihydro-cyclopentapyrazole; 3-(1H-Tetrazol-5-yl)-2,6-dihydro-4H-furo[3,4-c]pyrazole; 5-Ethyl-3-(1H-tetrazol-5-yl)-2,4,5,6-tetrahydro-cyclopentapyrazole; and 5-(5-Isopropyl-1H-pyrazol-3-yl)-1H-tetrazole; or a pharmaceutically acceptable salt thereof.

The invention further provides a method of reducing flushing induced by niacin or a niacin analog in a subject, comprising administering to said subject an effective flush reducing amount of a niacin receptor partial agonist and an effective lipid altering amount of niacin or a niacin analog. In one embodiment, said flushing is induced by niacin and in another embodiment, said flushing is induced by a niacin analog. In one embodiment, said niacin analog is a structural analog of niacin and in another embodiment, said niacin analog is a functional analog of niacin. In a further embodiment, said lipid altering amount of niacin or a niacin analog is at least 500 mg per day. In one embodiment, said niacin receptor partial agonist comprises a compound of Formula (I):

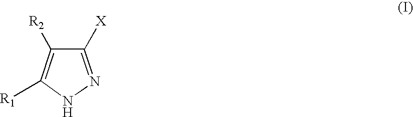

or a pharmaceutically acceptable salt thereof, wherein: X is a carboxyl or a tetrazol-5-yl group; R₁ is iso-propyl, 3-fluoro-benzyl, 3-chloro-benzyl, or 3-bromo-benzyl; and R₂ is H; or R₁ and R₂ together with the two pyrazole ring carbons to which they are bonded form a 5-membered carbocyclic ring optionally substituted with ethyl or a 5-membered heterocyclic ring optionally substituted with methyl. For example, said niacin receptor partial agonist can comprise a compound selected from the group consisting of: 3-(1H-Tetrazol-5-yl)-1,4,5,6-tetrahydro-cyclopentapyrazole; 5-(3-Fluoro-benzyl)-1H-pyrazole-3-carboxylic acid; 5-(3-Chloro-benzyl)-1H-pyrazole-3-carboxylic acid; 5-(3-Bromo-benzyl)-1H-pyrazole-3-carboxylic acid; 6-Methyl-3-(1H-tetrazol-5-yl)-4,6-dihydro-1H-furo[3,4-c]pyrazole; 3-(1H-Tetrazol-5-yl)-4,6-dihydro-1H-thieno[3,4-c]pyrazole; 3-(1H-Tetrazol-5-yl)-1,4-dihydro-cyclopentapyrazole; 3-(1H-Tetrazol-5-yl)-1,6-dihydro-cyclopentapyrazole; 3-(1H-Tetrazol-5-yl)-2,6-dihydro-4H-furo[3,4-c]pyrazole; 5-Ethyl-3-(1H-tetrazol-5-yl)-2,4,5,6-tetrahydro-cyclopentapyrazole; and 5-(5-Isopropyl-1H-pyrazol-3-yl)-1H-tetrazole; or a pharmaceutically acceptable salt thereof.

In addition, the invention provides a method of reducing flushing induced by niacin or a niacin analog in a subject, comprising administering to said subject an effective flush reducing amount of a niacin receptor partial agonist and subsequently administering to said subject an effective lipid altering amount of niacin or a niacin analog. In one embodiment, said flushing is induced by niacin and in another embodiment, said flushing is induced by a niacin analog. In one embodiment, said niacin analog is a structural analog of niacin and in another embodiment, said niacin analog is a functional analog of niacin. In a further embodiment, said lipid altering amount of niacin or a niacin analog is at least 500 mg per day. In one embodiment, said niacin receptor partial agonist comprises a compound of Formula (I):

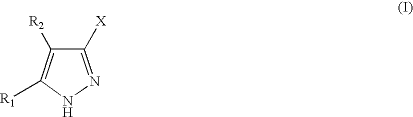

or a pharmaceutically acceptable salt thereof, wherein: X is a carboxyl or a tetrazol-5-yl group; R₁ is iso-propyl, 3-fluoro-benzyl, 3-chloro-benzyl, or 3-bromo-benzyl; and R₂ is H; or R₁ and R₂ together with the two pyrazole ring carbons to which they are bonded form a 5-membered carbocyclic ring optionally substituted with ethyl or a 5-membered heterocyclic ring optionally substituted with methyl. For example, said niacin receptor partial agonist can comprise a compound selected from the group consisting of: 3-(1H-Tetrazol-5-yl)-1,4,5,6-tetrahydro-cyclopentapyrazole; 5-(3-Fluoro-benzyl)-1H-pyrazole-3-carboxylic acid; 5-(3-Chloro-benzyl)-1H-pyrazole-3-carboxylic acid; 5-(3-Bromo-benzyl)-1H-pyrazole-3-carboxylic acid; 6-Methyl-3-(1H-tetrazol-5-yl)-4,6-dihydro-1H-furo[3,4-c]pyrazole; 3-(1H-Tetrazol-5-yl)-4,6-dihydro-1H-thieno[3,4-c]pyrazole; 3-(1H-Tetrazol-5-yl)-1,4-dihydro-cyclopentapyrazole; 3-(1H-Tetrazol-5-yl)-1,6-dihydro-cyclopentapyrazole; 3-(1H-Tetrazol-5-yl)-2,6-dihydro-4H-furo[3,4-c]pyrazole; 5-Ethyl-3-(1H-tetrazol-5-yl)-2,4,5,6-tetrahydro-cyclopentapyrazole; and 5-(5-Isopropyl-1H-pyrazol-3-yl)-1H-tetrazole; or a pharmaceutically acceptable salt thereof.

The invention also provides a method for preventing or treating a lipid-associated disorder in a subject, comprising administering to said subject an effective flush reducing amount of a niacin receptor partial agonist and an effective lipid altering amount of niacin or a niacin analog. In one embodiment, said flushing is induced by niacin and in another embodiment, said flushing is induced by a niacin analog. In one embodiment, said niacin analog is a structural analog of niacin and in another embodiment, said niacin analog is a functional analog of niacin. In a further embodiment, said lipid altering amount of niacin or a niacin analog is at least 500 mg per day. In one embodiment, said niacin receptor partial agonist comprises a compound of Formula (I):

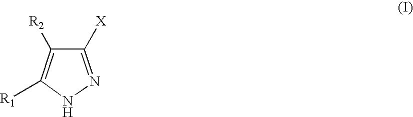

or a pharmaceutically acceptable salt thereof, wherein: X is a carboxyl or a tetrazol-5-yl group; R₁ is iso-propyl, 3-fluoro-benzyl, 3-chloro-benzyl, or 3-bromo-benzyl; and R₂ is H; or R₁ and R₂ together with the two pyrazole ring carbons to which they are bonded form a 5-membered carbocyclic ring optionally substituted with ethyl or a 5-membered heterocyclic ring optionally substituted with methyl. For example, said niacin receptor partial agonist can comprise a compound selected from the group consisting of: 3-(1H-Tetrazol-5-yl)-1,4,5,6-tetrahydro-cyclopentapyrazole; 5-(3-Fluoro-benzyl)-1H-pyrazole-3-carboxylic acid; 5-(3-Chloro-benzyl)-1H-pyrazole-3-carboxylic acid; 5-(3-Bromo-benzyl)-1H-pyrazole-3-carboxylic acid; 6-Methyl-3-(1H-tetrazol-5-yl)-4,6-dihydro-1H-furo[3,4-c]pyrazole; 3-(1H-Tetrazol-5-yl)-4,6-dihydro-1H-thieno[3,4-c]pyrazole; 3-(1H-Tetrazol-5-yl)-1,4-dihydro-cyclopentapyrazole; 3-(1H-Tetrazol-5-yl)-1,6-dihydro-cyclopentapyrazole; 3-(1H-Tetrazol-5-yl)-2,6-dihydro-4H-furo[3,4-c]pyrazole; 5-Ethyl-3-(1H-tetrazol-5-yl)-2,4,5,6-tetrahydro-cyclopentapyrazole; and 5-(5-Isopropyl-1H-pyrazol-3-yl)-1H-tetrazole; or a pharmaceutically acceptable salt thereof. In another embodiment, said method further comprises administering to said subject at least one agent selected from the group consisting of α-glucosidase inhibitor, aldose reductase inhibitor, biguanide, HMG-CoA reductase inhibitor, squalene synthesis inhibitor, fibrate, LDL catabolism enhancer, angiotensin converting enzyme inhibitor, insulin secretion enhancer and thiazolidinedione.

The invention further provides a method for preventing or treating a lipid-associated disorder in a subject, comprising administering to said subject an effective flush reducing amount of a niacin receptor partial agonist and subsequently administering to said subject an effective lipid altering amount of niacin or a niacin analog. In one embodiment, said flushing is induced by niacin and in another embodiment, said flushing is induced by a niacin analog. In one embodiment, said niacin analog is a structural analog of niacin and in another embodiment, said niacin analog is a functional analog of niacin. In a further embodiment, said lipid altering amount of niacin or a niacin analog is at least 500 mg per day. In one embodiment, said niacin receptor partial agonist comprises a compound of Formula (I):

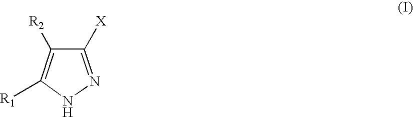

or a pharmaceutically acceptable salt thereof, wherein: X is a carboxyl or a tetrazol-5-yl group; R₁ is iso-propyl, 3-fluoro-benzyl, 3-chloro-benzyl, or 3-bromo-benzyl; and R₂ is H; or R₁ and R₂ together with the two pyrazole ring carbons to which they are bonded form a 5-membered carbocyclic ring optionally substituted with ethyl or a 5-membered heterocyclic ring optionally substituted with methyl. For example, said niacin receptor partial agonist can comprise a compound selected from the group consisting of: 3-(1H-Tetrazol-5-yl)-1,4,5,6-tetrahydro-cyclopentapyrazole; 5-(3-Fluoro-benzyl)-1H-pyrazole-3-carboxylic acid; 5-(3-Chloro-benzyl)-1H-pyrazole-3-carboxylic acid; 5-(3-Bromo-benzyl)-1H-pyrazole-3-carboxylic acid; 6-Methyl-3-(1H-tetrazol-5-yl)-4,6-dihydro-1H-furo[3,4-c]pyrazole; 3-(1H-Tetrazol-5-yl)-4,6-dihydro-1H-thieno[3,4-c]pyrazole; 3-(1H-Tetrazol-5-yl)-1,4-dihydro-cyclopentapyrazole; 3-(1H-Tetrazol-5-yl)-1,6-dihydro-cyclopentapyrazole; 3-(1H-Tetrazol-5-yl)-2,6-dihydro-4H-furo[3,4-c]pyrazole; 5-Ethyl-3-(1H-tetrazol-5-yl)-2,4,5,6-tetrahydro-cyclopentapyrazole; and 5-(5-Isopropyl-1H-pyrazol-3-yl)-1H-tetrazole; or a pharmaceutically acceptable salt thereof. In another embodiment, said method further comprises administering to said subject at least one agent selected from the group consisting of α-glucosidase inhibitor, aldose reductase inhibitor, biguanide, HMG-CoA reductase inhibitor, squalene synthesis inhibitor, fibrate, LDL catabolism enhancer, angiotensin converting enzyme inhibitor, insulin secretion enhancer and thiazolidinedione.

In addition, the invention provides a composition for administration of an effective lipid altering amount of niacin or a niacin analog having reduced capacity to provoke a flushing reaction in a subject, comprising (a) an effective lipid altering amount of niacin or a niacin analog, and (b) an effective flush reducing amount of a niacin receptor partial agonist. In one embodiment, said composition comprises an effective lipid altering amount of niacin and in another embodiment, said composition comprises an effective lipid altering amount of a niacin analog. In one embodiment, said niacin analog is a structural analog of niacin and in another embodiment, said niacin analog is a functional analog of niacin. In a further embodiment, said lipid altering amount of niacin or a niacin analog is at least 500 mg per day. In one embodiment, said niacin receptor partial agonist comprises a compound of Formula (I):

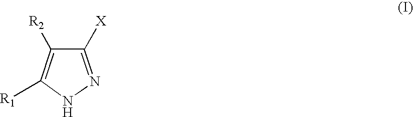

or a pharmaceutically acceptable salt thereof, wherein: X is a carboxyl or a tetrazol-5-yl group; R₁ is iso-propyl, 3-fluoro-benzyl, 3-chloro-benzyl, or 3-bromo-benzyl; and R₂ is H; or R₁ and R₂ together with the two pyrazole ring carbons to which they are bonded form a 5-membered carbocyclic ring optionally substituted with ethyl or a 5-membered heterocyclic ring optionally substituted with methyl. For example, said niacin receptor partial agonist can comprise a compound selected from the group consisting of: 3-(1H-Tetrazol-5-yl)-1,4,5,6-tetrahydro-cyclopentapyrazole; 5-(3-Fluoro-benzyl)-1H-pyrazole-3-carboxylic acid; 5-(3-Chloro-benzyl)-1H-pyrazole-3-carboxylic acid; 5-(3-Bromo-benzyl)-1H-pyrazole-3-carboxylic acid; 6-Methyl-3-(1H-tetrazol-5-yl)-4,6-dihydro-1H-furo[3,4-c]pyrazole; 3-(1H-Tetrazol-5-yl)-4,6-dihydro-1H-thieno[3,4-c]pyrazole; 3-(1H-Tetrazol-5-yl)-1,4-dihydro-cyclopentapyrazole; 3-(1H-Tetrazol-5-yl)-1,6-dihydro-cyclopentapyrazole; 3-(1H-Tetrazol-5-yl)-2,6-dihydro-4H-furo[3,4-c]pyrazole; 5-Ethyl-3-(1H-tetrazol-5-yl)-2,4,5,6-tetrahydro-cyclopentapyrazole; and 5-(5-Isopropyl-1H-pyrazol-3-yl)-1H-tetrazole; or a pharmaceutically acceptable salt thereof. In another embodiment, said composition further comprises at least one agent selected from the group consisting of α-glucosidase inhibitor, aldose reductase inhibitor, biguanide, HMG-CoA reductase inhibitor, squalene synthesis inhibitor, fibrate, LDL catabolism enhancer, angiotensin converting enzyme inhibitor, insulin secretion enhancer and thiazolidinedione.

The invention also provides a kit for preventing or treating a lipid-associated disorder comprising at least one dosage unit of a niacin receptor partial agonist and at least one dosage unit of niacin or a niacin analog, wherein said niacin receptor partial agonist is present in an amount effective to reduce flushing induced by niacin or a niacin analog in said subject and wherein said niacin or niacin analog is present in a lipid altering amount. In one embodiment, said kit comprises a dosage unit of niacin and in another embodiment, said kit comprises a dosage unit of a niacin analog. In one embodiment, said niacin analog is a structural analog of niacin and in another embodiment, said niacin analog is a functional analog of niacin. In a further embodiment, said dosage unit of niacin or a niacin analog is at least 500 mg per day. In one embodiment, said niacin receptor partial agonist comprises a compound of Formula (I):

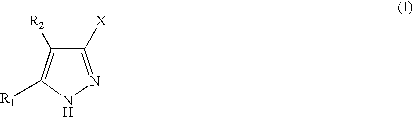

or a pharmaceutically acceptable salt thereof, wherein: X is a carboxyl or a tetrazol-5-yl group; R₁ is iso-propyl, 3-fluoro-benzyl, 3-chloro-benzyl, or 3-bromo-benzyl; and R₂ is H; or R₁ and R₂ together with the two pyrazole ring carbons to which they are bonded form a 5-membered carbocyclic ring optionally substituted with ethyl or a 5-membered heterocyclic ring optionally substituted with methyl. For example, said niacin receptor partial agonist can comprise a compound selected from the group consisting of: 3-(1H-Tetrazol-5-yl)-1,4,5,6-tetrahydro-cyclopentapyrazole; 5-(3-Fluoro-benzyl)-1H-pyrazole-3-carboxylic acid; 5-(3-Chloro-benzyl)-1H-pyrazole-3-carboxylic acid; 5-(3-Bromo-benzyl)-1H-pyrazole-3-carboxylic acid; 6-Methyl-3-(1H-tetrazol-5-yl)-4,6-dihydro-1H-furo[3,4-c]pyrazole; 3-(1H-Tetrazol-5-yl)-4,6-dihydro-1H-thieno[3,4-c]pyrazole; 3-(1H-Tetrazol-5-yl)-1,4-dihydro-cyclopentapyrazole; 3-(1H-Tetrazol-5-yl)-1,6-dihydro-cyclopentapyrazole; 3-(1H-Tetrazol-5-yl)-2,6-dihydro-4H-furo[3,4-c]pyrazole; 5-Ethyl-3-(1H-tetrazol-5-yl)-2,4,5,6-tetrahydro-cyclopentapyrazole; and 5-(5-Isopropyl-1H-pyrazol-3-yl)-1H-tetrazole; or a pharmaceutically acceptable salt thereof. In another embodiment, said kit further comprises at least one agent selected from the group consisting of α-glucosidase inhibitor, aldose reductase inhibitor, biguanide, HMG-CoA reductase inhibitor, squalene synthesis inhibitor, fibrate, LDL catabolism enhancer, angiotensin converting enzyme inhibitor, insulin secretion enhancer and thiazolidinedione.

The invention further provides a kit for preventing or treating a lipid-associated disorder comprising at least one dosage unit of a niacin receptor partial agonist and at least one separate dosage unit of niacin or a niacin analog, wherein said niacin receptor partial agonist is present in an amount effective to reduce flushing induced by niacin or a niacin analog in said subject and wherein said niacin or niacin analog is present in a lipid altering amount. In one embodiment, said kit comprises a dosage unit of niacin and in another embodiment, said kit comprises a dosage unit of a niacin analog. In one embodiment, said niacin analog is a structural analog of niacin and in another embodiment, said niacin analog is a functional analog of niacin. In a further embodiment, said dosage unit of niacin or a niacin analog is at least 500 mg per day. In one embodiment, said niacin receptor partial agonist comprises a compound of Formula (I):

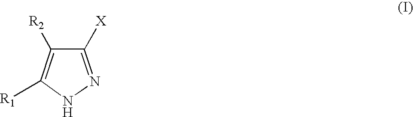

or a pharmaceutically acceptable salt thereof, wherein: X is a carboxyl or a tetrazol-5-yl group; R₁ is iso-propyl, 3-fluoro-benzyl, 3-chloro-benzyl, or 3-bromo-benzyl; and R₂ is H; or R₁ and R₂ together with the two pyrazole ring carbons to which they are bonded form a 5-membered carbocyclic ring optionally substituted with ethyl or a 5-membered heterocyclic ring optionally substituted with methyl. For example, said niacin receptor partial agonist can comprise a compound selected from the group consisting of: 3-(1H-Tetrazol-5-yl)-1,4,5,6-tetrahydro-cyclopentapyrazole; 5-(3-Fluoro-benzyl)-1H-pyrazole-3-carboxylic acid; 5-(3-Chloro-benzyl)-1H-pyrazole-3-carboxylic acid; 5-(3-Bromo-benzyl)-1H-pyrazole-3-carboxylic acid; 6-Methyl-3-(1H-tetrazol-5-yl)-4,6-dihydro-1H-furo[3,4-c]pyrazole; 3-(1H-Tetrazol-5-yl)-4,6-dihydro-1H-thieno[3,4-c]pyrazole; 3-(1H-Tetrazol-5-yl)-1,4-dihydro-cyclopentapyrazole; 3-(1H-Tetrazol-5-yl)-1,6-dihydro-cyclopentapyrazole; 3-(1H-Tetrazol-5-yl)-2,6-dihydro-4H-furo[3,4-c]pyrazole; 5-Ethyl-3-(1H-tetrazol-5-yl)-2,4,5,6-tetrahydro-cyclopentapyrazole; and 5-(5-Isopropyl-1H-pyrazol-3-yl)-1H-tetrazole; or a pharmaceutically acceptable salt thereof. In another embodiment, said kit further comprises at least one agent selected from the group consisting of α-glucosidase inhibitor, aldose reductase inhibitor, biguanide, HMG-CoA reductase inhibitor, squalene synthesis inhibitor, fibrate, LDL catabolism enhancer, angiotensin converting enzyme inhibitor, insulin secretion enhancer and thiazolidinedione.

In addition, the invention provides a kit for preventing or treating a lipid-associated disorder comprising at least one pre-dosage unit of a niacin receptor partial agonist and at least one separate dosage unit of niacin or a niacin analog, wherein said niacin receptor partial agonist is present in an amount effective to reduce flushing induced by niacin or a niacin analog in said subject and wherein said niacin or niacin analog is present in a lipid altering amount. In one embodiment, said kit comprises a dosage unit of niacin and in another embodiment, said kit comprises a dosage unit of a niacin analog. In one embodiment, said niacin analog is a structural analog of niacin and in another embodiment, said niacin analog is a functional analog of niacin. In a further embodiment, said dosage unit of niacin or a niacin analog is at least 500 mg per day. In one embodiment, said niacin receptor partial agonist comprises a compound of Formula (I):

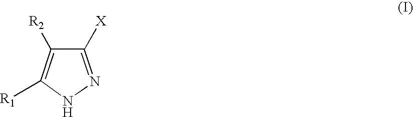

or a pharmaceutically acceptable salt thereof, wherein: X is a carboxyl or a tetrazol-5-yl group; R₁ is iso-propyl, 3-fluoro-benzyl, 3-chloro-benzyl, or 3-bromo-benzyl; and R₂ is H; or R₁ and R₂ together with the two pyrazole ring carbons to which they are bonded form a 5-membered carbocyclic ring optionally substituted with ethyl or a 5-membered heterocyclic ring optionally substituted with methyl. For example, said niacin receptor partial agonist can comprise a compound selected from the group consisting of: 3-(1H-Tetrazol-5-yl)-1,4,5,6-tetrahydro-cyclopentapyrazole; 5-(3-Fluoro-benzyl)-1H-pyrazole-3-carboxylic acid; 5-(3-Chloro-benzyl)-1H-pyrazole-3-carboxylic acid; 5-(3-Bromo-benzyl)-1H-pyrazole-3-carboxylic acid; 6-Methyl-3-(1H-tetrazol-5-yl)-4,6-dihydro-1H-furo[3,4-c]pyrazole; 3-(1H-Tetrazol-5-yl)-4,6-dihydro-1H-thieno[3,4-c]pyrazole; 3-(1H-Tetrazol-5-yl)-1,4-dihydro-cyclopentapyrazole; 3-(1H-Tetrazol-5-yl)-1,6-dihydro-cyclopentapyrazole; 3-(1H-Tetrazol-5-yl)-2,6-dihydro-4H-furo[3,4-c]pyrazole; 5-Ethyl-3-(1H-tetrazol-5-yl)-2,4,5,6-tetrahydro-cyclopentapyrazole; and 5-(5-Isopropyl-1H-pyrazol-3-yl)-1H-tetrazole; or a pharmaceutically acceptable salt thereof. In another embodiment, said kit further comprises at least one agent selected from the group consisting of α-glucosidase inhibitor, aldose reductase inhibitor, biguanide, HMG-CoA reductase inhibitor, squalene synthesis inhibitor, fibrate, LDL catabolism enhancer, angiotensin converting enzyme inhibitor, insulin secretion enhancer and thiazolidinedione.

### DETAILED DESCRIPTION

Applicants have discovered that niacin receptor partial agonists can significantly reduce flushing induced by niacin or a niacin analog. As disclosed herein, administration of niacin receptor partial agonists to mice significantly reduced flushing induced by niacin (see examples 1 and 2). These mice still had the ability to flush as shown by administration of PGD₂ (see Example 2). In addition, as disclosed herein, a niacin receptor partial agonist which reduced flushing did not interfere with niacin-induced reduction of free fatty acid release (see Example 3).

Although niacin has been used as a therapy for lipid-associated disorders for several years, the receptor through which niacin acted was not known until recently. Initially, it was suggested that niacin may act through a specific GPCR (Lorenzen A, et al. (2001) Molecular Pharmacology 59:349-357). Eventually, a known orphan GPCR called HM74a was identified as the nicotinic acid receptor (see, for example, U.S. application Ser. No. 10/314,048). The nucleotide sequence of the human niacin receptor can be found at GenBank Accession No. NM_{—}177551 and herein as SEQ ID NO:1.

The invention provides a method of reducing flushing induced by niacin or a niacin analog in a subject, comprising administering to said subject an effective flush reducing amount of a niacin receptor partial agonist. In one embodiment, said flushing is induced by niacin and in another embodiment, said flushing is induced by a niacin analog. In one embodiment, said niacin analog is a structural analog of niacin and in another embodiment, said niacin analog is a functional analog of niacin. In a further embodiment, flushing is completely reduced or eliminated. In one embodiment, said niacin receptor partial agonist comprises a compound of Formula (I):

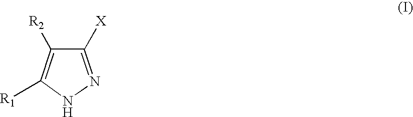

or a pharmaceutically acceptable salt thereof, wherein: X is a carboxyl or a tetrazol-5-yl group; R₁ is iso-propyl, 3-fluoro-benzyl, 3-chloro-benzyl, or 3-bromo-benzyl; and R₂ is H; or R₁ and R₂ together with the two pyrazole ring carbons to which they are bonded form a 5-membered carbocyclic ring optionally substituted with ethyl or a 5-membered heterocyclic ring optionally substituted with methyl. For example, said niacin receptor partial agonist can comprise a compound selected from the group consisting of: 3-(1H-Tetrazol-5-yl)-1,4,5,6-tetrahydro-cyclopentapyrazole; 5-(3-Fluoro-benzyl)-1H-pyrazole-3-carboxylic acid; 5-(3-Chloro-benzyl)-1H-pyrazole-3-carboxylic acid; 5-(3-Bromo-benzyl)-1H-pyrazole-3-carboxylic acid; 6-Methyl-3-(1H-tetrazol-5-yl)-4,6-dihydro-1H-furo[3,4-c]pyrazole; 3-(1H-Tetrazol-5-yl)-4,6-dihydro-1H-thieno[3,4-c]pyrazole; 3-(1H-Tetrazol-5-yl)-1,4-dihydro-cyclopentapyrazole; 3-(1H-Tetrazol-5-yl)-1,6-dihydro-cyclopentapyrazole; 3-(1H-Tetrazol-5-yl)-2,6-dihydro-4H-furo[3,4-c]pyrazole; 5-Ethyl-3-(1H-tetrazol-5-yl)-2,4,5,6-tetrahydro-cyclopentapyrazole; and 5-(5-Isopropyl-1H-pyrazol-3-yl)-1H-tetrazole; or a pharmaceutically acceptable salt thereof.

In general, flushing can be caused by several means, for example, flushing can be induced by social stress or anxiety, hormonal changes, heat, or holding one's breath, all of which can result in a transient flushing of the face. The subject application is related to flushing induced by niacin or a niacin analog.

As used herein, the term “flushing induced by niacin or a niacin analog” means a detectable cutaneous flushing reaction caused by administration of a sufficient dose of niacin or a niacin analog. A flushing reaction is characterized by redness of the skin and can also include other symptoms, for example, cutaneous itching, tingling, a feeling of warmth, or headache. The flushing reaction can occur anywhere on the skin, for example, on the face, neck or trunk, and can occur in one location or at more than one location. In humans, the flushing reaction can last from several minutes to a several hours. Generally, in humans a flushing reaction caused by oral administration of sufficient doses of niacin or a niacin analog can last anywhere from 20 minutes to 8 hours or more. In a mouse or rat, the flushing reaction usually peaks at about 3 minutes post administration (by injection) of niacin and has declined significantly after about 30 minutes.

In any of the embodiments of the invention, when niacin or a niacin analog induces flushing, it is present in a dose sufficient to cause detectable flushing. The amount of niacin or a niacin analog required to produce a detectable flushing reaction depends on several variables, for example, the formulation of the compound and the individual subject. In particular, the amount of niacin or a niacin analog required to produce a detectable flushing reaction can be dependent on, for example, the body weight of the individual, genetic makeup of the individual or general health of the individual. Amounts of niacin or a niacin analog that can cause a flushing reaction in a human can be less than those required to lower the amount of atherosclerosis associated serum lipids and can include, for example, at least 175 mg per day, at least 200 mg per day, at least 250 mg per day, at least 500 mg per day, at least 750 mg per day, at least 1 g per day, at least 1.5 g per day, at least 2 g per day, at least 2.5 g per day, at least 3 g per day, at least 3.5 g per day, at least 4 g per day, at least 4.5 g per day, at least 5 g per day, at least 5.5 g per day, at least 6 g per day, at least 6.5 g per day, at least 7 g per day, at least 7.5 g per day, at least 8 g per day, at least 8.5 g per day, at least 9 g per day, or more. For example, 500 mg to 2 g or more per day of niacin can cause a flushing reaction in most humans.

As used herein a “subject” means any animal, including mammals, for example, mice, rats, other rodents, rabbits, dogs, cats, swine, cattle, sheep, horses, or primates, for example, humans. In one embodiment, a subject is a human.

As used herein, “niacin” means nicotinic acid which has the following chemical formula:

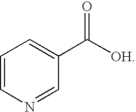

As understood by one skilled in the art, niacin can be formulated with other compounds such that its pharmacologic properties are modified. For example, niacin can be formulated as an immediate release (IR) form or as an extended or sustained release (SR) form depending on other compounds that are added to the niacin. In one embodiment, niacin is the IR form. In one embodiment, niacin is not a single dose once a day extended release form of niacin.

Extended or sustained release formulations are designed to slowly release the active ingredient from the tablet or capsule, which allows a reduction in dosing frequency as compared to the typical dosing frequency associated with conventional or immediate dosage forms. The slow drug release is designed to reduce and prolong blood levels of the drug and, thus, minimize or lessen the flushing side effects that are associated with conventional or immediate release niacin products. However, studies in patients with lipid-associated disorders have demonstrated that some extended or sustained release products do not have the same advantageous lipid-altering effects as immediate release niacin, and in fact have a worse side effect profile compared to the immediate release product. For example, extended or sustained release niacin formulations are known to cause greater incidences of liver toxicity, as described in Henken et al.: Am J Med, 91:1991 (1991) and Dalton et al.: Am J Med, 93: 102 (1992). Extended or sustained release formulations of niacin have been developed, such as Nicobid® capsules (Rhone-Poulenc Rorer), Endur-acin® (Innovite Corporation), and the formulations described in U.S. Pat. Nos. 5,126,145 and 5,268,181, which describe sustained release niacin formulations containing two different types of hydroxy propyl methylcelluloses and a hydrophobic component.

As used herein, “niacin analog” means a compound structurally or functionally related to, but distinct from, niacin. For example, a niacin analog can be structurally related to niacin. Several structural analogs of niacin are known in the art and examples are described herein. In some embodiments, structural analogues of niacin contain at least one functional acidic group, such as carboxyl, tetrazolyl, and the like. In some embodiments, structural analogues of niacin contain at least one nitrogen ring atom, such as the nitrogen present in pyridinyl, pyrazolyl, isoxazolyl, and the like. In some embodiments, structural analogues of niacin contain at least one functional acidic group and at least one nitrogen ring atom. These groups include pro-drug groups that are transformed in vivo to yield the functional acidic group or ring nitrogen, for example, by hydrolysis in blood. A thorough discussion is provided in T. Higuchi and V. Stella, “Pro-drugs as Novel Delivery Systems,” Vol. 14 of the A.C.S. Symposium Series, and in “Bioreversible Carriers in Drug Design,” ed. Edward B. Roche, American Pharmaceutical Association and Pergamon Press, 1987, both of which are hereby incorporated by reference.

A niacin analog can be functionally related to niacin, for example, a niacin analog can have a function of niacin such as specifically binding to the niacin receptor or initiating an intracellular signal in response to binding at the niacin receptor. For example, in any of the embodiments of the invention, a niacin analog can be a niacin receptor agonist. A niacin analog can be either a structural or functional analog of niacin, or a niacin analog can be both a structural and functional analog of niacin.

Several analogs or derivatives of niacin are known in the art and can be found, for example, in Merck Index, An Encyclopedia of Chemicals, Drugs, and Biologicals, Tenth Edition (1983). Niacin analogs can include, for example, nicotinyl alcohol tartrate, d-glucitol hexanicotinate, aluminum nicotinate, niceritrol, d, 1-alpha-tocopheryl nicotinate, 6-OH-nicotinic acid, nicotinaria acid, nicotinamide, nicotinamide-N-oxide, 6-OH-nicotinamide, NAD, N-methyl-2-pyriidine-8-carboxamide, N-methyl-nicotinamide, N-ribosyl-2-pyridone-5-carboxide, N-methyl-4-pyridone-5-carboxamide, bradilian, sorbinicate, hexanicite, ronitol, and lower alcohol esters of nicotinic acid. As described above for niacin, niacin analogs can be formulated in different ways to modify their pharmacologic properties.

As described above, niacin analogs include niacin receptor agonists (other than niacin). Several niacin receptor agonists are known in the art and can be found, for example, in Merck Index, An Encyclopedia of Chemicals, Drugs, and Biologicals, Tenth Edition (1983). Specific examples of niacin agonists, which are considered niacin analogs herein, are listed in the embodiments below and in the following patent applications: 60/418,057 and 60/478,664, which are incorporated herein in their entirety.

In some embodiments, a niacin analog of the present invention is of the following chemical formula:

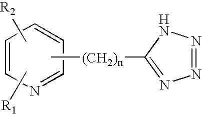

wherein:
R₁ is selected from the group consisting of halogen, hydroxyl, acetylamino, amino, alkoxy, carboalkoxy, alkylthio, monoalkylamino, dialkylamino, N-alkylcarbamyl, N,N-dialkylcarbamyl, alkylsulfonyl, said alkyl groups containing from 1 to 4 carbons, trifluoromethyl, trifluoromethoxy, trifluoromethylthio, methoxymethyl, carboxy, carbamyl, alkanoyloxy containing up to 4 carbon atoms, phenyl, p-chlorophenyl, p-methylphenyl and p-aminophenyl;
R₂ is selected from the group consisting of halogen, alkannoyloxy containing from 1-4 carbon atoms, carboalkoxy containing from 2 to 5 carbon atoms, carbamyl, N-alkyl carbamyl and N,N-dialkylcarbamyl wherein said alkyl groups contain from 1-4 carbon atoms and trifluoromethyl; and
n is a whole number from 0 to 4; or
N-oxides thereof.

In some embodiments, a niacin analog of the present invention is of the following chemical formula:

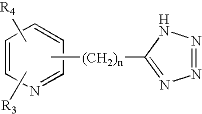

wherein:
R₃ and R₄ are hydrogen, alkyl containing from 1 to 4 carbon atoms or cycloalkyl containing from 3 to 7 carbon atoms; and
n is a whole number from 0 to 4; or
N-oxides thereof.

In some embodiments, a niacin analog of the present invention is of the following chemical formula:

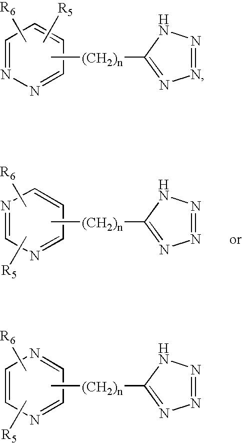

wherein:
R₅ and R₅ are each selected from the group consisting of H, halogen, hydroxyl, amino, alkyloxy, alkylthio, monoalkylamino, dialkylamino, N-alkylcarbamyl, N,N-dialkylcarbamyl, alkylsulfoxy, alkylsulfony, said alkyl groups containing from 1 to 4 carbons, trifluoromethyl, trifluoromethoxy, trifluoromethylthio, carboxy, carbamyl, alkanoyloxy containing up to 4 carbon atoms, phenyl, p-chlorophenyl, p-methylphenyl and p-aminophenyl; and
n is a whole number from 0 to 4; or
N-oxides thereof.

In some embodiments, a niacin analog of the present invention is of the following chemical formula:

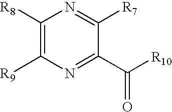

wherein:
at least one of R₇, R₈ and R₉ is C₁₋₆ alkyl and the others are hydrogen atoms; R₁₀ is hydroxy or C₁₋₆ alkoxy, or a salt of the compounds when R₁₀ is hydroxy with a pharmaceutically acceptable base; or a 4-N-oxide thereof. The position of the N-oxide is designated by the following numbering and a structure for a 4-N-oxide has the following structure:

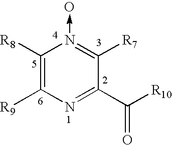

One particular 4-N-oxide is 5-Methylpyrazine-2-carboxylic acid-4-oxide (Acipimox™) and has the structure:

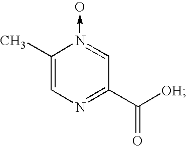

In some embodiments, a niacin analog of the present invention is of the following chemical formula:

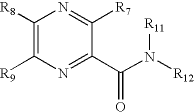

wherein:
at least one of R₇, R₈ and R₉ is C₁₋₆ alkyl and the others are hydrogen atoms; and each of R₁₁ and R₁₂, which may be the same or different, is hydrogen or C₁₋₆ alkyl; or a 4-N-oxide thereof; the position of the N-oxide is the same as described above herein;

In some embodiments, a niacin analog of the present invention is of the following chemical formula:

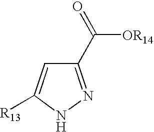

wherein:
at least one of R₁₃ represents an alkyl group of 7-11 carbon atoms and R₁₄ represents H or a lower alkyl group of up to two carbon atoms, and a pharmaceutically acceptable carrier;

In some embodiments, a niacin analog of the present invention is Pyrazine-2-carboxylic acid amide and has the structure:

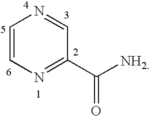

In some embodiments, a niacin analog of the present invention is 5-chloro-pyrazine-2-carboxylic acid amide and has the structure:

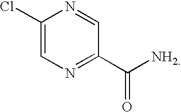

In some embodiments, a niacin analog of the present invention is 5-amino-pyrazine-2-carboxylic acid amide and has the structure:

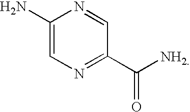

In some embodiments, a niacin analog of the present invention is 5-benzyl-pyrazine-2-carboxylic acid amide and has the structure:

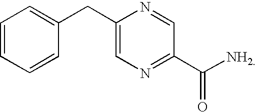

In some embodiments, a niacin analog of the present invention is 6-chloro-pyrazine-2-carboxylic acid amide and has the structure:

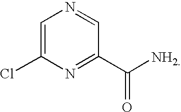

In some embodiments, a niacin analog of the present invention is 6-methoxy-pyrazine-2-carboxylic acid amide and has the structure:

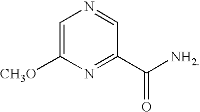

In some embodiments, a niacin analog of the present invention is 3-chloro-pyrazine-2-carboxylic acid amide and has the structure:

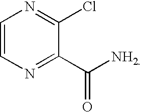

In some embodiments, a niacin analog of the present invention is 3-methoxy-pyrazine-2-carboxylic acid amide and has the structure:

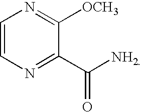

In some embodiments, a niacin analog of the present invention is pyrazine-2-carboxylic acid ethylamide and has the structure:

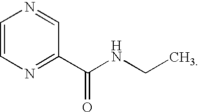

In some embodiments, a niacin analog of the present invention is morpholin-4-yl-pyrzine-2-ylmethanone and has the structure:

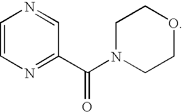

In some embodiments, a niacin analog of the present invention is 5-methyl-pyrazine-2-carboxylic acid (6-methyl-pyrazin-2-yl)-amide and has the structure:

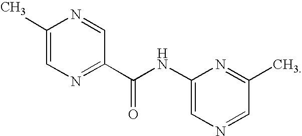

In some embodiments, a niacin analog of the present invention is 5-methyl-pyrazine-2-carboxylic acid (5-methyl-pyrazin-2-yl)-amide and has the structure:

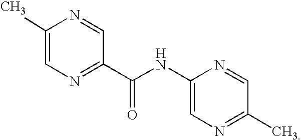

In some embodiments, a niacin analog of the present invention is 5-methyl-pyrazine-2-carboxylic acid (3-methyl-pyrazin-2-yl)-amide and has the structure:

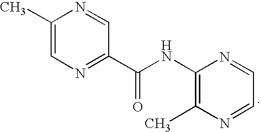

In some embodiments, a niacin analog of the present invention is (5-methyl-pyrazin-2-yl)-morpholin-4-yl-methanone and has the structure:

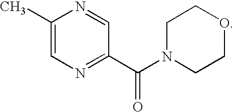

In some embodiments, a niacin analog of the present invention is 5-methyl-pyrazine-2-carboxylic acid hydroxyamide and has the structure:

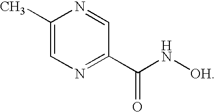

In some embodiments, a niacin analog of the present invention is pyrazine-2-carboxylic acid and has the structure:

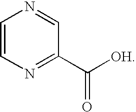

In some embodiments, a niacin analog of the present invention is 5-amino-pyrazine-2-carboxylic acid and has the structure:

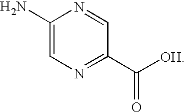

In some embodiments, a niacin analog of the present invention is 5-benzyl-pyrazine-2-carboxylic acid and has the structure:

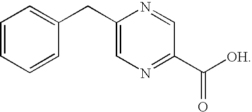

In some embodiments, a niacin analog of the present invention is 6-chloro-pyrazine-2-carboxylic acid and has the structure:

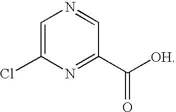

In some embodiments, a niacin analog of the present invention is 6-methoxy-pyrazine-2-carboxylic acid and has the structure:

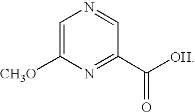

In some embodiments, a niacin analog of the present invention is 3-hydroxy-pyrazine-2-carboxylic acid and has the structure:

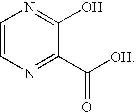

In some embodiments, a niacin analog of the present invention is 5-methyl-pyrazine-2-carboxylic acid 2-hydroxy-ethyl ester and has the structure:

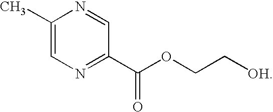

In some embodiments, a niacin analog of the present invention is 5-methyl-pyrazine-2-carboxylic acid allyl ester and has the structure:

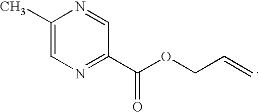

In some embodiments, a niacin analog of the present invention is 5-methyl-pyrazine-2-carboxylic acid phenyl ester and has the structure:

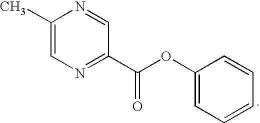

In some embodiments, a niacin analog of the present invention is 5-methyl-pyrazine-2-carboxylic acid ethoxycarbonylmethyl ester and has the structure:

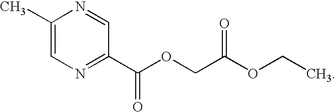

In some embodiments, a niacin analog of the present invention is pyrazine-2-carboxylic acid methyl ester and has the structure:

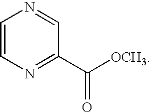

In some embodiments, a niacin analog of the present invention is 2-methyl-5-(1H-tetrazol-5-yl)-pyrazine and has the structure:

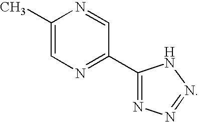

or 4-N-oxides thereof as described above herein.

In some embodiments, a niacin analog of the present invention is 5-(5-Methyl-isoxazol-3-yl)-1H-tetrazole and has the structure:

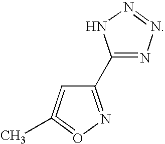

In some embodiments, a niacin analog of the present invention is 5-(3-Methyl-isoxazol-5-yl)-1H-tetrazole and has the structure:

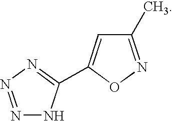

In some embodiments, a niacin analog of the present invention is 5-(3-Quinolyl)tetrazole and has the structure:

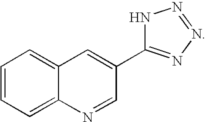

In some embodiments, a niacin analog of the present invention is Nicotinic acid and has the structure:

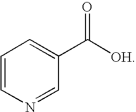

In some embodiments, a niacin analog of the present invention is Pyridazine-4-carboxylic acid and has the structure:

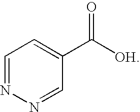

In some embodiments, a niacin analog of the present invention is 3-Pyridine acetic acid and has the structure:

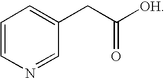

In some embodiments, a niacin analog of the present invention is 5-Methylnicotinic acid and has the structure:

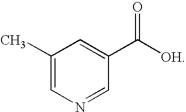

In some embodiments, a niacin analog of the present invention is 6-Methylnicotinic acid and has the structure:

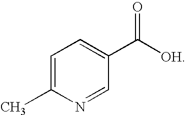

In some embodiments, a niacin analog of the present invention is Nicotinic acid-1-oxide and has the structure:

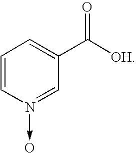

In some embodiments, a niacin analog of the present invention is 2-Hydroxynicotinic acid and has the structure:

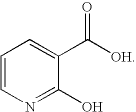

In some embodiments, a niacin analog of the present invention is Furane-3-carboxylic acid and has the structure:

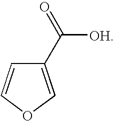

In some embodiments, a niacin analog of the present invention is 3-Methylisoxazole-5-carboxylic acid and has the structure:

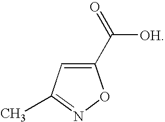

In some embodiments, niacin analogs of the present invention are of the following chemical formula:

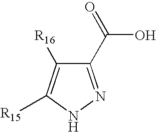

wherein:
R₁₅ is selected from the group consisting of isopropyl, n-propyl, n-butyl, n-undecyl, phenyl, 3-chlorophenyl, 4-chlorophenyl, benzyl, 4-benyzyl, 4-methoxybenzyl, 2-phenylethyl, and 3-phenylpropyl; and
R₁₆ is H; or
R₁₅ and R₁₆ together form a —OCH₂CH₂—, —C₃H₆—, or —C₄H₈— group provided that the oxygen atom of said —OCH₂CH₂— group is bonded to the 5 position of the pyrazole ring.

Since a niacin analog includes a functional analog of niacin, a niacin analog includes a niacin receptor agonist. Therefore, the invention also provides a method of reducing flushing induced by niacin or a niacin receptor agonist in a subject, comprising administering to said subject an effective flush reducing amount of a niacin receptor partial agonist.

Generally, when a ligand binds with its receptor, often referred to as activation of the receptor, there is a change in the conformation of the receptor that facilitates coupling between the intracellular region and an intracellular G-protein. Although other G proteins exist, currently, Gq, Gs, Gi, Gz and Go are G proteins that have been identified. There are also promiscuous G proteins, which appear to couple several classes of GPCRs to the phospholipase C pathway, such as Gα15 or Gα16 [Offermanns & Simon, J Biol Chem (1995) 270:15175-80], or chimeric G proteins designed to couple a large number of different GPCRs to the same pathway [Milligan & Rees, Trends in Pharmaceutical Sciences (1999) 20:118-24]. Ligand-activated GPCR coupling with the G-protein initiates a signaling cascade process referred to as signal transduction. Under normal conditions, signal transduction ultimately results in cellular activation or cellular inhibition.

Under physiological conditions, GPCRs exist in the cell membrane in equilibrium between two different conformations: an inactive state and an active state. A receptor in an inactive state is unable to link to the intracellular signaling transduction pathway to initiate signal transduction leading to a biological response. Changing the receptor conformation to the active state allows linkage to the transduction pathway (via the G-protein) and produces a biological response. A receptor may be stabilized in an active state by a ligand or a compound such as a drug. In addition, recent discoveries provide means other than ligands or drugs to promote and stabilize the receptor in the active state conformation. These means effectively stabilize the receptor in an active state by simulating the effect of a ligand binding to the receptor. Stabilization by such ligand-independent means is termed constitutive receptor activation.

The initiation of an intracellular signal can be determined, for example, through the measurement of the level of a second messenger such as cyclic AMP (cAMP), cyclic GMP (cGMP), inositol triphosphate (IP3), diacylglycerol (DAG), and calcium. Several assays are well known in the art for measuring these second messengers, for example, the FLIPR assay, the melanophore assay, or CRE-reporter assay (see for example, Examples 7, 10, 11, and 12 herein).

An agonist is material, for example, a ligand or candidate compound, that activates an intracellular response when it binds to the receptor. An intracellular response can be, for example, enhancement of GTP binding to membranes or modulation of the level of a second messenger such as cAMP or IP3. In some embodiments, an agonist is material not previously known to activate the intracellular response when it binds to the receptor (for example, to enhance GTPγS binding to membranes or to lower intracellular cAMP level). A partial agonist is material, for example, a ligand or candidate compound, which activate an intracellular response when it binds to the receptor but to a lesser degree or extent than do full agonists.

As used herein, a “niacin receptor partial agonist” is material that activates an intracellular response when it binds to a niacin receptor, but to a lesser degree than niacin which is a full agonist at the niacin receptor. Technically, the term partial agonist is a relative term because a partial agonist generates a partial response compared to a full agonist. Since new compounds are being discovered with time, the full agonist can change and a formerly full agonist can become a partial agonist. For clarity, a niacin receptor partial agonist as used herein is compared to niacin as the full agonist. A niacin receptor partial agonist has a detectably lesser degree of activation of an intracellular response compared to the niacin, i.e. a niacin receptor partial agonist elicits less than a maximal response. Thus, a niacin receptor partial agonist has less efficacy than niacin. For example, a niacin receptor partial agonist has 90% or less efficacy compared to niacin, 85% or less efficacy compared to niacin, 80% or less efficacy compared to niacin, 75% or less efficacy compared to niacin, 70% or less efficacy compared to niacin, 65% or less efficacy compared to niacin, 60% or less efficacy compared to niacin, 55% or less efficacy compared to niacin, 50% or less efficacy compared to niacin, 45% or less efficacy compared to niacin, 40% or less efficacy compared to niacin, 35% or less efficacy compared to niacin, 30% or less efficacy compared to niacin, 25% or less efficacy compared to niacin, 20% or less efficacy compared to niacin, 15% or less efficacy compared to niacin, or 10% efficacy compared to niacin. For example, a niacin receptor partial agonist can have 10% to 90% efficacy compared to niacin, 20% to 80% efficacy compared to niacin, 30% to 70% efficacy compared to niacin, 40% to 60% efficacy compared to niacin, or 45% to 55% efficacy compared to niacin. Efficacy, which is the magnitude of the measured response, is different from potency which is the amount of compound it takes to elicit a defined response. Therefore, a niacin receptor partial agonist can be more, less, or equally potent when compared to an agonist, antagonist, or inverse agonist.

A niacin receptor partial agonist can be determined using assays well known in the art and disclosed herein. For example, a niacin receptor partial agonist can be determined using a cAMP assay.

Representative niacin receptor partial agonists are shown in Table A:

TABLE A

| Compound No. | Structure | Chemical Name |
|---|---|---|
| 1 | 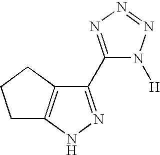 | 3-(1H-Tetrazol-5-yl)-1,4,5,6-tetrahydro-cyclopentapyrazole |
| 2 | 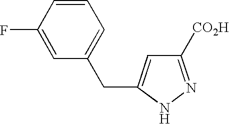 | 5-(3-Fluoro-benzyl)-1H-pyrazole-3-carboxylic acid |
| 3 | 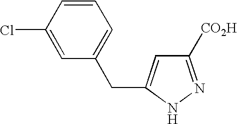 | 5-(3-Chloro-benzyl)-1H-pyrazole-3-carboxylic acid |
| 4 | 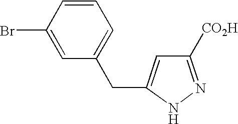 | 5-(3-Bromo-benzyl)-1H-pyrazole-3-carboxylic acid |
| 5 | 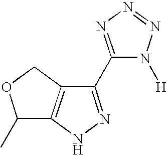 | 6-Methyl-3-(1H-tetrazol-5-yl)-4,6-dihydro-1H-furo[3,4-c]pyrazole |
| 6 | 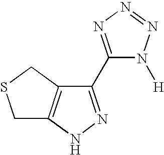 | 3-(1H-Tetrazol-5-yl)-4,6-dihydro-1H-thieno[3,4-c]pyrazole |
| 7 | 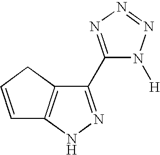 | 3-(1H-Tetrazol-5-yl)-1,4-dihydro-cyclopentapyrazole |
| 8 | 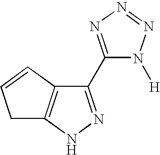 | 3-(1H-Tetrazol-5-yl)-1,6-dihydro-cyclopentapyrazole |
| 9 | 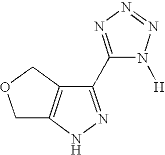 | 3-(1H-Tetrazol-5-yl)-2,6-dihydro-4H-furo[3,4-c]pyrazole |
| 10 | 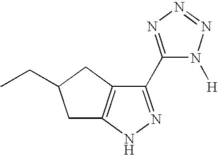 | 5-Ethyl-3-(1H-tetrazol-5-yl)-2,4,5,6-tetrahydro-cyclopentapyrazole |
| 11 | 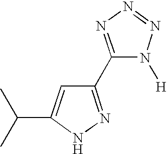 | 5-(5-Isopropyl-1H-pyrazol-3-yl)-1H-tetrazole |

Compounds of the present invention may exist in various tautomeric forms. It is well appreciated to those of skill in the art that pyrazoles can exist in at least two tautomeric forms and although Formula (I) represents one form it is understood that all tautomeric forms are embraced by the present invention. By way of illustration, two possible tautomers for the pyrazole in Formula (I) are shown below:

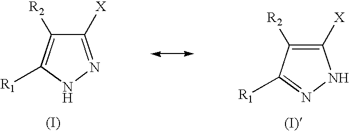

In addition, for Formula (I) when X is a tetrazol-5-yl group, it is also well appreciated to those of skill in the art that tetrazoles can exist in at least two tautomeric forms, it is understood that all tautomeric forms for the tetrazole group are embraced by the present invention. By way of illustration, two possible tautomers for Formula (I) when X is a tetrazol-5-yl group are shown below:

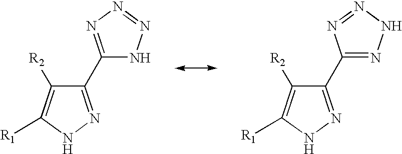

Further, it is understood that when X is a tetrazol-5-yl group then tautomers can exist for both pyrazole ring and also the tetrazole ring in combination. It is understood that all tautomers that can exist for the compounds disclosed herein are within the scope of the invention.

The term “carboxy” or “carboxyl” denotes the group —CO₂H and the corresponding conjugate base —CO₂⁻; also referred to as a carboxylic acid group.

The term “5-membered carbocyclic ring” denotes a non-aromatic ring containing 5 ring carbons and optionally one or two endocyclic ring double bonds, in some embodiments two ring carbons of the 5-membered carbocyclic ring are shared with the pyrazole ring; for example, but not limited to, when R₁ and R₂ together with the two pyrazole ring carbons to which they are bonded form a 5-membered carbocyclic ring with the following chemical structures:

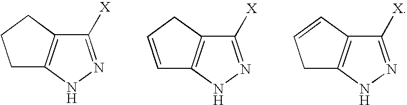

The term “5-membered heterocyclic ring” denotes a non-aromatic ring containing 4 ring carbons and one heteroatom selected from oxygen and sulfur, and optionally one endocyclic ring double bond. In some embodiments two ring carbons of the 5-membered carbocyclic ring are shared with the pyrazole ring; for example, but not limited to, when R₁ and R₂ together with the two pyrazole ring carbons to which they are bonded form a 5-membered heterocyclic ring with the following chemical structures:

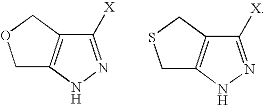

The term “tetrazol-5-yl” refers to the group as shown below and the corresponding tautomers:

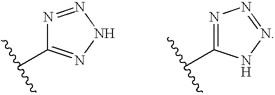

Regarding the niacin receptor, several niacin receptor sequences are known in the art. For example, a human niacin receptor nucleotide sequence can be found at GenBank Accession No. NM_{—}177551 and is listed herein as SEQ ID NO:1. It is also understood that limited modifications to the niacin receptor can be made without destroying the ability of a niacin receptor to bind niacin. For example, niacin receptor is intended to include other niacin receptor polypeptides, for example, species homologues of the human niacin receptor polypeptide (SEQ ID NO: 2). The sequence of species homologs of the human niacin receptor are present in the database, for example, a rat homolog of the niacin receptor can be found in GenBank at Accession No. BAC58009. In addition, a niacin receptor includes splice variants and allelic variants of niacin receptors that retain substantially the niacin receptor binding function of the entire niacin receptor polypeptide.

Further, a niacin receptor can contain amino acid changes, for example, conservative amino acid changes, compared to the wild-type receptor so long as the mutated receptor retains substantially the niacin receptor binding function of the wild-type niacin receptor polypeptide. Conservative and non-conservative amino acid changes, gaps, and insertions to an amino acid sequence can be compared to a reference sequence using available algorithms and programs such as the Basic Local Alignment Search Tool (“BLAST”) using default settings (See, e.g., Karlin and Altschul, Proc Natl Acad Sci USA (1990) 87:2264-8; Altschul et al., J Mol Biol (1990) 215:403-410; Altschul et al., Nature Genetics (1993) 3:266-72; and Altschul et al., Nucleic Acids Res (1997) 25:3389-3402).

The niacin receptor specifically binds to niacin. The term specifically binds is intended to mean the polypeptide will have an affinity for a target polypeptide that is measurably higher than its affinity for an un-related polypeptide. Several methods for detecting or measuring receptor binding are well known in the art, for example, radio-ligand binding assays, or assays with a functional read-out such as a FLIPR assay.

It is understood that a fragment of a niacin receptor which retains substantially the niacin receptor binding function of the entire polypeptide can be used in lieu of the entire polypeptide. For example, a ligand binding domain of a niacin receptor can be used in lieu of the entire polypeptide in order to determine binding of a partial agonist to a niacin receptor.

As used herein, an “effective flush reducing amount” of a niacin receptor partial agonist means an amount sufficient to cause a reduction in flushing induced by niacin or a niacin analog.

As used herein “reducing” means a decrease in a measurable quantity or a particular activity and is used synonymously with the terms “decreasing”, “diminishing”, “lowering”, and “lessening.” In reference to an amount of flushing, a reduction in flushing can be, for example, a decrease in flushing or the elimination of flushing. For example, flushing can be reduced at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%. In addition, flushing can be reduced 100% or eliminated such that no flushing is detectable. In one embodiment, flushing is reduced at least about 80%. In another embodiment, the reduction of flushing is a complete reduction or elimination of flushing.

Several methods can be used to detect and quantify flushing. For example, flushing can be visually detected and quantified. One method for detecting and quantifying flushing is by Laser Doppler, for example using a Pirimed PimII Laser Dopler. In addition, surveys of subjects can be taken to assess flushing and the severity of symptoms that can be associated with flushing such as tingling or a feeling of warmth. Another method for detecting and quantifying flushing can include measurement of the level of prostaglandin D₂ (PGD₂) or prostaglandin F₂ (PGF₂) in a biological sample from a subject such as blood or urine. In addition, for example, the level of PGD-M, the major urinary metabolite of PGD₂ can be measured from the urine of subjects. Assays for measuring prostaglandin levels are commercially available, for example, an enzyme immunoassay for PGD₂ is available from Cayman Chemical (Ann Arbor, Mich.).

As understood by one skilled in the art, the amount of niacin or niacin analog required to achieve a reduction in flushing will vary, for example, with the specific compound, its formulation, route of administration, and the individual subject.

Suitable routes of administration to a subject include oral, topical, nasal, rectal, transmucosal, or intestinal administration, parenteral delivery, including intra-muscular, subcutaneous, intramedullary injections, as well as intrathecal, direct intra-ventricular, intravenous, intraperitoneal, intranasal, intrapulmonary (inhaled) or intra-ocular injections using methods known in the art. Other routes of administration are aerosol and depot formulation. In one embodiment, route of administration is oral.

The invention further provides a method of reducing flushing induced by niacin or a niacin analog in a subject, comprising administering to said subject an effective flush reducing amount of a niacin receptor partial agonist and an effective lipid altering amount of niacin or a niacin analog. In one embodiment, said flushing is induced by niacin and in another embodiment, said flushing is induced by a niacin analog. In a further embodiment, flushing is completely reduced or eliminated. In one embodiment, said niacin analog is a structural analog of niacin and in another embodiment, said niacin analog is a functional analog of niacin. In a further embodiment, said lipid altering amount of niacin or a niacin analog is at least 500 mg per day. In one embodiment, said niacin receptor partial agonist comprises a compound of Formula (I):

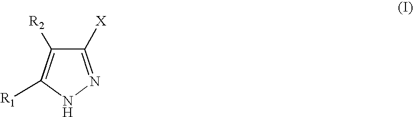

or a pharmaceutically acceptable salt thereof, wherein: X is a carboxyl or a tetrazol-5-yl group; R₁ is iso-propyl, 3-fluoro-benzyl, 3-chloro-benzyl, or 3-bromo-benzyl; and R₂ is H; or R₁ and R₂ together with the two pyrazole ring carbons to which they are bonded form a 5-membered carbocyclic ring optionally substituted with ethyl or a 5-membered heterocyclic ring optionally substituted with methyl. For example, said niacin receptor partial agonist can comprise a compound selected from the group consisting of: 3-(1H-Tetrazol-5-yl)-1,4,5,6-tetrahydro-cyclopentapyrazole; 5-(3-Fluoro-benzyl)-1H-pyrazole-3-carboxylic acid; 5-(3-Chloro-benzyl)-1H-pyrazole-3-carboxylic acid; 5-(3-Bromo-benzyl)-1H-pyrazole-3-carboxylic acid; 6-Methyl-3-(1H-tetrazol-5-yl)-4,6-dihydro-1H-furo[3,4-c]pyrazole; 3-(1H-Tetrazol-5-yl)-4,6-dihydro-1H-thieno[3,4-c]pyrazole; 3-(1H-Tetrazol-5-yl)-1,4-dihydro-cyclopentapyrazole; 3-(1H-Tetrazol-5-yl)-1,6-dihydro-cyclopentapyrazole; 3-(1H-Tetrazol-5-yl)-2,6-dihydro-4H-furo[3,4-c]pyrazole; 5-Ethyl-3-(1H-tetrazol-5-yl)-2,4,5,6-tetrahydro-cyclopentapyrazole; and 5-(5-Isopropyl-1H-pyrazol-3-yl)-1H-tetrazole; or a pharmaceutically acceptable salt thereof.

As used herein the term “effective lipid altering amount” in reference to an amount of niacin or a niacin analog means an amount of these compounds sufficient to detectably alter the amount of an atherosclerosis associated serum lipid, for example, a decrease in the amount of LDL-cholesterol, VLDL-cholesterol, or triglycerides or an increase in HDL-cholesterol in a subject. For example, an effective lipid altering amount of niacin can increase the amount of HDL-cholesterol or lower the amount of LDL-cholesterol. In addition, for example, an effective lipid altering amount of niacin can both increase the amount of HDL-cholesterol and lower the amount of LDL-cholesterol. Standard laboratory assays for measuring the amount of these lipids in the blood are well known in the art. (see, for example, Example 6 herein).

Cholesterol is transported in the blood by lipoprotein complexes, such as VLDL-cholesterol, LDL-cholesterol, and high density lipoprotein-cholesterol (HDL-cholesterol). LDL carries cholesterol in the blood to the subendothelial spaces of blood vessel walls. It is believed that peroxidation of LDL-cholesterol within the subendothelial space of blood vessel walls leads to atherosclerosis plaque formation. HDL-cholesterol, on the other hand, is believed to counter plaque formation and delay or prevent the onset of cardiovascular disease and atherosclerotic symptoms. Several subtypes of HDL-cholesterol, such as HDL₁-cholesterol, HDL₂-cholesterol and HDL₃-cholesterol, have been identified to date.

There are several mechanisms by which HDL may protect against the progression of atherosclerosis. Studies in vitro have shown that HDL is capable of removing cholesterol from cells [Picardo et al., (1986) Arteriosclerosis, 6, 434-441]. Data of this nature suggest that one antiatherogenic property of HDL may lie in its ability to deplete tissue of excess free cholesterol and eventually lead to the delivery of this cholesterol to the liver [Glomset, (1968) J. Lipid Res., 9, 155-167]. This has been supported by experiments showing efficient transfer of cholesterol from HDL to the liver [Glass et al., (1983) J. Biol. Chem., 258 7161-7167; McKinnon et al., (1986) J. Biol. Chem., 26, 2548-2552]. In addition, HDL may serve as a reservoir in the circulation for apoproteins necessary for the rapid metabolism of triglyceride-rich lipoproteins (Grow and Fried, (1978) J. Biol. Chem., 253, 1834-1841; Lagocki and Scanu, (1980) J. Biol. Chem., 255, 3701-3706; Schaefer et al., J. Lipid Res., (1982) 23, 1259-1273].

Generally, the total cholesterol/HDL-cholesterol (i.e., TC/HDL) ratio can represent a useful predictor as to the risk of an individual in developing a condition, such as atherosclerosis, heart disease or stroke. The current classification of plasma lipid levels is shown in Table B:

TABLE B
CLASSIFICATION OF PLASMA LIPID LEVELS

| | | | |
|---|---|---|---|
| TOTAL CHOLESTEROL | <200 | mg/dl | Desirable |
| | 200-239 | mg/dl | Borderline High |
| | >240 | mg/dl | High |
| HDL-CHOLESTEROL | <40 | mg/dl | Low (Men) |
| | <50 | mg/dl | Low (Women) |
| | >60 | mg/dl | High |

From: 2001 National Cholesterol Education Program Guidelines

Accordingly, the recommended total cholesterol/HDL-C (i.e., TC/HDL) ratio indicates that a ratio of less than or equal to 3.5 is ideal and a ratio of greater than 4.5 is considered “at risk.” The value of determining the TC/HDL ratio is clearly evident in the circumstance where an individual presents with “normal” LDL and total cholesterol but possesses low HDL-cholesterol. Based on LDL and total cholesterol the individual may not qualify for treatment, however, when factoring in the HDL-cholesterol level, a more accurate risk assessment can be obtained. Thus, if the individual's level of HDL-cholesterol is such that the ratio is greater than 4.5 then therapeutic or prophylactic intervention can be warranted.

Regarding LDL-cholesterol levels, the American Heart Association currently considers an LDL-cholesterol level of less than 100 mg/dL as optimal, 100-129 mg/dL is near optimal, 130-159 mg/dL is borderline high, 160-189 mg/dL is high and 190 mg/dL is considered a very high level of LDL-cholesterol. Regarding triglyceride levels, the American Heart Association currently considers less than 150 mg/L as normal, 150-199 mg/dL is borderline-high, 200-499 mg/dL is high and 500 mg/dL is considered a very high level of triglycerides.

The amount of niacin or niacin analog required in order to alter the amount of atherosclerosis associated serum lipids will vary with the formulation of the compound and the individual. In particular, the amount of niacin or niacin analog required to alter the amount of atherosclerosis associated serum lipids can be dependent on, for example, the body weight of the individual, genetic makeup of the individual, or the general health of the individual. Amounts of niacin or a niacin analog that can alter the amount of atherosclerosis associated serum lipids can include, for example, at least 500 mg per day, at least 750 mg per day, at least 1 g per day, at least 1.5 g per day, at least 2 g per day, at least 2.5 g per day, at least 3 g per day, at least 3.5 g per day, at least 4 g per day, at least 4.5 g per day, at least 5 g per day, at least 5.5 g per day, at least 6 g per day, at least 6.5 g per day, at least 7 g per day, at least 7.5 g per day, at least 8 g per day, at least 8.5 g per day, at least 9 g per day, or more. In one embodiment, said lipid altering amount of niacin or a niacin analog is at least 500 mg of niacin per day. In another embodiment, said lipid altering amount of niacin or a niacin analog is 1 to 3 grams per day.

In addition, the invention provides a method of reducing flushing induced by niacin or a niacin analog in a subject, comprising administering to said subject an effective flush reducing amount of a niacin receptor partial agonist and subsequently administering to said subject an effective lipid altering amount of niacin or a niacin analog. In one embodiment, said flushing is induced by niacin and in another embodiment, said flushing is induced by a niacin analog. In a further embodiment, flushing is completely reduced or eliminated. In a yet further embodiment, said lipid altering amount of niacin or a niacin analog is at least 500 mg per day. In one embodiment, said niacin receptor partial agonist comprises a compound of Formula (I):

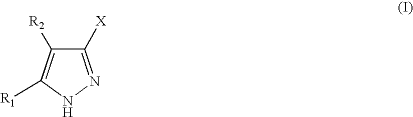

or a pharmaceutically acceptable salt thereof, wherein: X is a carboxyl or a tetrazol-5-yl group; R₁ is iso-propyl, 3-fluoro-benzyl, 3-chloro-benzyl, or 3-bromo-benzyl; and R₂ is H; or R₁ and R₂ together with the two pyrazole ring carbons to which they are bonded form a 5-membered carbocyclic ring optionally substituted with ethyl or a 5-membered heterocyclic ring optionally substituted with methyl. For example, said niacin receptor partial agonist can comprise a compound selected from the group consisting of: 3-(1H-Tetrazol-5-yl)-1,4,5,6-tetrahydro-cyclopentapyrazole; 5-(3-Fluoro-benzyl)-1H-pyrazole-3-carboxylic acid; 5-(3-Chloro-benzyl)-1H-pyrazole-3-carboxylic acid; 5-(3-Bromo-benzyl)-1H-pyrazole-3-carboxylic acid; 6-Methyl-3-(1H-tetrazol-5-yl)-4,6-dihydro-1H-furo[3,4-c]pyrazole; 3-(1H-Tetrazol-5-yl)-4,6-dihydro-1H-thieno[3,4-c]pyrazole; 3-(1H-Tetrazol-5-yl)-1,4-dihydro-cyclopentapyrazole; 3-(1H-Tetrazol-5-yl)-1,6-dihydro-cyclopentapyrazole; 3-(1H-Tetrazol-5-yl)-2,6-dihydro-4H-furo[3,4-c]pyrazole; 5-Ethyl-3-(1H-tetrazol-5-yl)-2,4,5,6-tetrahydro-cyclopentapyrazole; and 5-(5-Isopropyl-1H-pyrazol-3-yl)-1H-tetrazole; or a pharmaceutically acceptable salt thereof.

For the methods disclosed herein, the niacin receptor partial agonist and niacin or niacin analog can be administered together or separately, at same time or different times. For example, niacin or a niacin analog can be combined in the same formulation as a niacin receptor partial agonist or can be a separate formulation. If the niacin or niacin analog and niacin receptor partial agonist are separate formulations they can be administered together or separately, for example, separated by less than a minute such as if taken at the same sitting or separated by a greater amount of time such as if taken at different sittings.

In the method of the invention where the niacin receptor partial agonist is administered to the subject and then subsequently the niacin or a niacin analog is administered, the time between administration of the niacin receptor partial agonist and the subsequent administration of the niacin or niacin analog can be, for example, at least about 1 minute, at least about 5 minutes, at least about 10 minutes, at least about 20 minutes, at least about 30 minutes, at least about 45 minutes, at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 12 hours, at least about 14 hours, at least about 20 hours or at least about 24 hours or more.

The invention also provides a method for preventing or treating a lipid-associated disorder in a subject, comprising administering to said subject an effective flush reducing amount of a niacin receptor partial agonist and an effective lipid altering amount of niacin or a niacin analog. In one embodiment, said flushing is induced by niacin and in another embodiment, said flushing is induced by a niacin analog. In one embodiment, said niacin analog is a structural analog of niacin and in another embodiment, said niacin analog is a functional analog of niacin. In a further embodiment, flushing is completely reduced or eliminated. In a yet further embodiment, said lipid altering amount of niacin or a niacin analog is at least 500 mg per day. In one embodiment, said niacin receptor partial agonist comprises a compound of Formula (I):

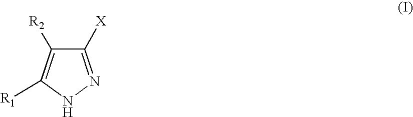

or a pharmaceutically acceptable salt thereof, wherein: X is a carboxyl or a tetrazol-5-yl group; R₁ is iso-propyl, 3-fluoro-benzyl, 3-chloro-benzyl, or 3-bromo-benzyl; and R₂ is H; or R₁ and R₂ together with the two pyrazole ring carbons to which they are bonded form a 5-membered carbocyclic ring optionally substituted with ethyl or a 5-membered heterocyclic ring optionally substituted with methyl. For example, said niacin receptor partial agonist can comprise a compound selected from the group consisting of: 3-(1H-Tetrazol-5-yl)-1,4,5,6-tetrahydro-cyclopentapyrazole; 5-(3-Fluoro-benzyl)-1H-pyrazole-3-carboxylic acid; 5-(3-Chloro-benzyl)-1H-pyrazole-3-carboxylic acid; 5-(3-Bromo-benzyl)-1H-pyrazole-3-carboxylic acid; 6-Methyl-3-(1H-tetrazol-5-yl)-4,6-dihydro-1H-furo[3,4-c]pyrazole; 3-(1H-Tetrazol-5-yl)-4,6-dihydro-1H-thieno[3,4-c]pyrazole; 3-(1H-Tetrazol-5-yl)-1,4-dihydro-cyclopentapyrazole; 3-(1H-Tetrazol-5-yl)-1,6-dihydro-cyclopentapyrazole; 3-(1H-Tetrazol-5-yl)-2,6-dihydro-4H-furo[3,4-c]pyrazole; 5-Ethyl-3-(1H-tetrazol-5-yl)-2,4,5,6-tetrahydro-cyclopentapyrazole; and 5-(5-Isopropyl-1H-pyrazol-3-yl)-1H-tetrazole; or a pharmaceutically acceptable salt thereof. In another embodiment, said method further comprises administering to said subject at least one agent selected from the group consisting of α-glucosidase inhibitor, aldose reductase inhibitor, biguanide, HMG-CoA reductase inhibitor, squalene synthesis inhibitor, fibrate, LDL catabolism enhancer, angiotensin converting enzyme inhibitor, insulin secretion enhancer and thiazolidinedione.

As used herein the term “treating” in reference to a disorder means a reduction in severity of one or more symptoms associated with a particular disorder. Therefore, treating a disorder does not necessarily mean a reduction in severity of all symptoms associated with a disorder and does not necessarily mean a complete reduction in the severity of one or more symptoms associated with a disorder. Similarly, the term “preventing” means prevention of the occurrence or onset of one or more symptoms associated with a particular disorder and does not necessarily mean the complete prevention of a disorder. The methods of the invention can be used to treat a niacin-responsive disorder including, for example, a lipid-associated disorder as described below.

As used herein the term “lipid-associated disorder” means any disorder related to a non-optimal level of an atherosclerosis associated serum lipid, for example, LDL-cholesterol, VLDL-cholesterol, HDL-cholesterol or triglycerides in a subject. Therefore, a lipid-associated disorder can be, for example, an elevated level of LDL-cholesterol, a reduced level of HDL-cholesterol, or disorders that are caused, at least in part, by a non-optimal level of an atherosclerosis associated serum lipid such as atherosclerosis, heart attack (myocardial infarction), or stroke. Optimal levels of atherosclerosis associated serum lipids were discussed above and non-optimal levels of these lipids or less than optimal ratios of these lipids are considered to be lipid-associated disorders.

Hyperlipidemia, which is a general term for elevated concentrations of any or all of the lipids in the plasma such as cholesterol, triglycerides and lipoproteins, is a lipid-associated disorder. Hypelipidemia can be acquired or can be congenital. Specific forms of hyperlipidemia can include, for example, hypercholesteremia, familial dysbetalipoproteinemia, diabetic dyslipidemia, nephrotic dyslipidemia and familial combined hyperlipidemia. Hypercholesteremia is characterized by an elevation in serum low density lipoprotein-cholesterol and serum total cholesterol. Familial dysbetalipoproteinemia, also known as Type III hyperlipidemia, is characterized by an accumulation of very low density lipoprotein-cholesterol (VLDL-cholesterol) particles called beta-VLDLs in the serum. Also associated with this condition, is a replacement of normal apolipoprotein E3 with abnormal isoform apolipoprotein E2. Diabetic dyslipidemia is characterized by multiple lipoprotein abnormalities, such as an overproduction of VLDL-cholesterol, abnormal VLDL triglyceride lipolysis, reduced LDL-cholesterol receptor activity and, on occasion, Type III hyperlipidemia. Nephrotic dyslipidemia is difficult to treat and frequently includes hypercholesteremia and hypertriglyceridemia. Familial combined hyperlipidemia is characterized by multiple phenotypes of hyperlipidemia, i.e., Type IIa, IIb, IV, V or hyperapobetalipoproteinemia.

Disorders that are caused, at least in part, by a non-optimal level of an atherosclerosis associated serum lipid are included in the definition of a lipid-associated disorder. Such disorders include, for example, coronary artery disease (CAD) or coronary heart disease, congestive heart failure, angina, aneurysm, ischemic heart disease, myocardial infarction and stroke. A lipid-associated disorder can include heart disease such as coronary heart disease, which are disorders comprising a narrowing of the small blood vessels that supply blood to the heart and congestive heart failure where the heart loses its ability to pump blood efficiently. A lipid-associated disorder can include a disorder caused by reduced blood flow to a tissue or organ due to partial or complete blockage of a blood vessel. Such disorders include, for example, angina, ischemic heart disease, myocardial infarction and stroke. A lipid-associated disorder can include a disorder caused by weakened blood vessels such as, for example, an aneurysm, which is a weakened area in a blood vessel often caused by atherosclerosis.

The methods, compositions and kits of the invention can be used to prevent or treat a lipid-associated disorder in a subject. When used to prevent a lipid-associated disorder, the subject can have optimal levels of lipids but may be at risk for a lipid-associated disorder for other reason, for example, a family history of a lipid-associated disorder. The methods, composition and kits of the invention can be used prophylactically to prevent a lipid-associated disorder in a subject of any age, for example, in a child or adult with obesity or diabetes which are risk factors for developing a lipid-associated disorder.

The invention also provides methods for combination therapy which includes another therapeutic compound or compounds in addition to a niacin receptor partial agonist and niacin or a niacin analog. Other therapeutic compounds can include, for example, compounds that can be used to further reduce flushing or compounds that can be used to further lower the amount of atherosclerosis associated serum lipids in a subject.

Therapeutic compounds that can be combined with a niacin receptor partial agonist and niacin or a niacin analog can include, for example, compounds that reduce prostaglandin synthesis, such as PGD₂ synthesis. Such compounds can include, for example, non-steroidal anti-inflammatory drugs (NSAIDs). Examples of NSAIDS include: aspirin, salicylate salts, ibuprofen, indomethacin, naproxen, sodium naproxen, ketoprofen, fenoprofen, oxaprozin, sulindac, flurbiprofen, etodolac, diclofenac, ketorolac, tolmetin, nabumetone, suprofen, benoxaprofen, carprofen, aclofenac, fenclofenac, zomepirac, meclofenamate, mefanamic acid, oxyphenbutazone, phenylbutazone and piroxicam. In addition, to combinations with COX-1 inhibitors, the therapeutic compounds can be combined with selective COX-2 inhibitors such as Celecoxib or Rofecoxib.

Therapeutic compounds that can be combined with a niacin receptor partial agonist and niacin or a niacin analog can include, for example, compounds that lower the amount of atherosclerosis associated serum lipids in subjects. Such compounds include, for example, a α-glucosidase inhibitor, aldose reductase inhibitor, biguanide, HMG-CoA reductase inhibitor, squalene synthesis inhibitor, fibrate, LDL catabolism enhancer, angiotensin converting enzyme (ACE) inhibitor, insulin secretion enhancer and thiazolidinedione.

α-Glucosidase inhibitors belong to the class of drugs which competitively inhibit digestive enzymes such as α-amylase, maltase, α-dextrinase, sucrase, etc. in the pancreas and or small intesting. The reversible inhibition by α-glucosidase inhibitors retard, diminish or otherwise reduce blood glucose levels by delaying the digestion of starch and sugars. Some representative examples of α-glucosidase inhibitors include acarbose, N-(1,3-dihydroxy-2-propyl)valiolamine (generic name; voglibose), miglitol, and α-glucosidase inhibitors known in the art.

Aldose reductase inhibitors are drugs which inhibit the first-stage rate-limiting enzyme in the polyol pathway. Examples of the aldose reductase inhibitors include tolurestat; epalrestat; 3,4-dihydro-2,8-diisopropyl-3-thioxo-2H-1,4-benzoxazine-4-acetic acid; 2,7-difluorospiro(9H-fluorene-9,4′-imidazolidine)-2′,5′-dione (generic name: imirestat); 3-[(4-bromo-2-fluorophenyl)methy]-7-chloro-3,4-dihydro-2,4-dioxo-1(2H)-quinazoline acetic acid (generic name: zenarestat); 6-fluoro-2,3-dihydro-2′,5′-dioxo-spiro[4H-1-benzopyran-4,4′-imidazolidine]-2-carboxamide (SNK-860); zopolrestat; sorbinil; and 1-[(3-bromo-2-benzofuranyl)sulfonyl]-2,4-imidazolidinedione (M-16209), and aldose reductase inhibitors known in the art.

The biguanides are a class of drugs that stimulate anaerobic glycolysis, increase the sensitivity to insulin in the peripheral tissues, inhibit glucose absorption from the intestine, suppress of hepatic gluconeogenesis, and inhibit fatty acid oxidation. Examples of biguanides include phenformin, metformin, buformin, and biguanides known in the art.

Statin compounds belong to a class of drugs that lower blood cholesterol levels by inhibiting hydroxymethylglutalyl CoA (HMG-CoA) reductase. HMG-CoA reductase is the rate-limiting enzyme in cholesterol biosynthesis. A statin that inhibits this reductase lowers serum LDL concentrations by upregulating the activity of LDL receptors and responsible for clearing LDL from the blood. Examples of the statin compounds include rosuvastatin, pravastatin and its sodium salt, simvastatin, lovastatin, atorvastatin, fluvastatin, cerivastatin, and HMG-CoA reductase inhibitors known in the art.

Squalene synthesis inhibitors belong to a class of drugs that lower blood cholesterol levels by inhibiting synthesis of squalene. Examples of the squalene synthesis inhibitors include (S)-α-[Bis[2,2-dimethyl-1-oxopropoxy)methoxy]phosphinyl]-3-phenoxybenzenebutanesulfonic acid, mono potassium salt (BMS-188494) and squalene synthesis inhibitors known in the art.

Fibrate compounds belong to a class of drugs that lower blood cholesterol levels by inhibiting synthesis and secretion of triglycerides in the liver and activating a lipoprotein lipase. Fibrates have been known to activate peroxisome proliferators-activated receptors and induce lipoprotein lipase expression. Examples of fibrate compounds include bezafibrate, beclobrate, binifibrate, ciplofibrate, clinofibrate, clofibrate, clofibric acid, etofibrate, fenofibrate, gemfibrozil, nicofibrate, pirifibrate, ronifibrate, simfibrate, theofibrate, and fibrates known in the art.

LDL (low-density lipoprotein) catabolism enhancers belong to a class of drugs that lower blood cholesterol levels by increasing the number of LDL receptors, examples include LDL catabolism enhancers known in the art.

Angiotensin converting enzyme (ACE) inhibitors belong to the class of drugs that partially lower blood glucose levels as well as lowering blood pressure by inhibiting angiotensin converting enzymes. Examples of the angiotensin converting enzyme inhibitors include captopril, enalapril, alacepril, delapril; ramipril, lisinopril, imidapril, benazepril, ceronapril, cilazapril, enalaprilat, fosinopril, moveltopril, perindopril, quinapril, spirapril, temocapril, trandolapril, and angiotensin converting enzyme inhibitors known in the art.

Insulin secretion enhancers belong to the class of drugs having the property to promote secretion of insulin from pancreatic β cells. Examples of the insulin secretion enhancers include sulfonylureas (SU). The sulfonylureas (SU) are drugs which promote secretion of insulin from pancreatic β cells by transmitting signals of insulin secretion via SU receptors in the cell membranes. Examples of the sulfonylureas include tolbutamide; chlorpropamide; tolazamide; acetohexamide; 4-chloro-N-[(1-pyrrolidinylamino)carbonyl]-benzenesulfonamide (generic name: glycopyramide) or its ammonium salt; glibenclamide (glyburide); gliclazide; 1-butyl-3-metanilylurea; carbutamide; glibonuride; glipizide; gliquidone; glisoxepid; glybuthiazole; glibuzole; glyhexamide; glymidine; glypinamide; phenbutamide; tolcyclamide, glimepiride, and other insulin secretion enhancers known in the art. Other insulin secretion enhancers include N-[[4-(1-methylethyl)cyclohexyl)carbonyl]-D-phenylalanine (Nateglinide); calcium (2S)-2-benzyl-3-(cis-hexahydro-2-isoindolinylcarbonyl)propionate dihydrate (Mitiglinide, KAD-1229); and other insulin secretion enhancers known in the art. Thiazolidinediones belong to the class of drugs more commonly known as TZDs. Examples of thiazolidinediones include rosiglitazone, pioglitazone, and thiazolidinediones known in the art.

The methods, kits and compositions of the invention can be useful, for example, to reduce flushing induced by niacin or a niacin analog. Niacin or a niacin analog can be administered to a subject, for example, in order to prevent or treat a niacin-responsive disorder. A niacin-responsive disorder is a disorder or disease that can be prevented or treated by niacin or a niacin analog. A niacin-responsive disorder can include, for example, a lipid-associated disorder as described herein. For example, a lipid-associated disorder can be a low amount of high density lipoprotein (HDL)-cholesterol, an elevated amount of low density lipoprotein (LDL)-cholesterol, an elevated amount of triglycerides, or a disorder that is caused, at least in part, by a non-optimal level of an atherosclerosis associated serum lipid such as atherosclerosis, heart disease or stroke.

Another example of a niacin responsive disorder is dysmenorrhea or painful menstruation. In one report, a group of 80 women suffering from painful menstrual cramps were supplemented with 100 mg of niacin twice daily, beginning 7 to 10 days before the onset of menses and then every 2 to 3 hours during heavy cramps [Hudgins, (1952) Am Pract Dig Treat 3:892-893; Hudgins (1954) West J Surg Obstet Gynecol 62:610-611]. About 90% of subjects experienced significant relief. The dosage required during heavy cramping (100 mg every 2 to 3 hours) is high enough to cause flushing in some women. In this case, the methods, kits and compositions of the invention can be of use to reduce flushing induced by niacin.

The invention further provides a method for preventing or treating a lipid-associated disorder in a subject, comprising administering to said subject an effective flush reducing amount of a niacin receptor partial agonist and subsequently administering to said subject an effective lipid altering amount of niacin or a niacin analog. In one embodiment, said flushing is induced by niacin and in another embodiment, said flushing is induced by a niacin analog. In one embodiment, said niacin analog is a structural analog of niacin and in another embodiment, said niacin analog is a functional analog of niacin. In a further embodiment, flushing is completely reduced or eliminated. In a yet further embodiment, said lipid altering amount of niacin or a niacin analog is at least 500 mg per day. In one embodiment, said niacin receptor partial agonist comprises a compound of Formula (I):

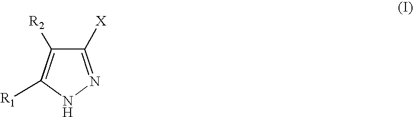

or a pharmaceutically acceptable salt thereof, wherein: X is a carboxyl or a tetrazol-5-yl group; R₁ is iso-propyl, 3-fluoro-benzyl, 3-chloro-benzyl, or 3-bromo-benzyl; and R₂ is H; or R₁ and R₂ together with the two pyrazole ring carbons to which they are bonded form a 5-membered carbocyclic ring optionally substituted with ethyl or a 5-membered heterocyclic ring optionally substituted with methyl. For example, said niacin receptor partial agonist can comprise a compound selected from the group consisting of: 3-(1H-Tetrazol-5-yl)-1,4,5,6-tetrahydro-cyclopentapyrazole; 5-(3-Fluoro-benzyl)-1H-pyrazole-3-carboxylic acid; 5-(3-Chloro-benzyl)-1H-pyrazole-3-carboxylic acid; 5-(3-Bromo-benzyl)-1H-pyrazole-3-carboxylic acid; 6-Methyl-3-(1H-tetrazol-5-yl)-4,6-dihydro-1H-furo[3,4-c]pyrazole; 3-(1H-Tetrazol-5-yl)-4,6-dihydro-1H-thieno[3,4-c]pyrazole; 3-(1H-Tetrazol-5-yl)-1,4-dihydro-cyclopentapyrazole; 3-(1H-Tetrazol-5-yl)-1,6-dihydro-cyclopentapyrazole; 3-(1H-Tetrazol-5-yl)-2,6-dihydro-4H-furo[3,4-c]pyrazole; 5-Ethyl-3-(1H-tetrazol-5-yl)-2,4,5,6-tetrahydro-cyclopentapyrazole; and 5-(5-Isopropyl-1H-pyrazol-3-yl)-1H-tetrazole; or a pharmaceutically acceptable salt thereof. In another embodiment, said method further comprises administering to said subject at least one agent selected from the group consisting of α-glucosidase inhibitor, aldose reductase inhibitor, biguanide, HMG-CoA reductase inhibitor, squalene synthesis inhibitor, fibrate, LDL catabolism enhancer, angiotensin converting enzyme inhibitor, insulin secretion enhancer and thiazolidinedione.

As described above, the time between administration of the niacin receptor partial agonist and the subsequent administration of the niacin or niacin analog can be, for example, at least about 1 minute, at least about 5 minutes, at least about 10 minutes, at least about 20 minutes, at least about 30 minutes, at least about 45 minutes, at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 12 hours, at least about 14 hours, at least about 20 hours or at least about 24 hours or more.

In addition, the invention provides a composition for administration of an effective lipid altering amount of niacin or a niacin analog having reduced capacity to provoke a flushing reaction in a subject, comprising (a) an effective lipid altering amount of niacin or a niacin analog, and (b) an effective flush reducing amount of a niacin receptor partial agonist. In one embodiment, said composition comprises an effective lipid altering amount of niacin and in another embodiment, said composition comprises an effective lipid altering amount of a niacin analog. In one embodiment, said niacin analog is a structural analog of niacin and in another embodiment, said niacin analog is a functional analog of niacin. In a further embodiment, said lipid altering amount of niacin or a niacin analog is at least 500 mg per day. In one embodiment, said niacin receptor partial agonist comprises a compound of Formula (I):

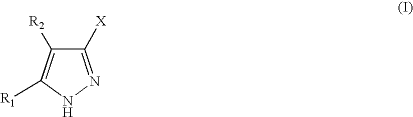

or a pharmaceutically acceptable salt thereof, wherein: X is a carboxyl or a tetrazol-5-yl group; R₁ is iso-propyl, 3-fluoro-benzyl, 3-chloro-benzyl, or 3-bromo-benzyl; and R₂ is H; or R₁ and R₂ together with the two pyrazole ring carbons to which they are bonded form a 5-membered carbocyclic ring optionally substituted with ethyl or a 5-membered heterocyclic ring optionally substituted with methyl. For example, said niacin receptor partial agonist can comprise a compound selected from the group consisting of: 3-(1H-Tetrazol-5-yl)-1,4,5,6-tetrahydro-cyclopentapyrazole; 5-(3-Fluoro-benzyl)-1H-pyrazole-3-carboxylic acid; 5-(3-Chloro-benzyl)-1H-pyrazole-3-carboxylic acid; 5-(3-Bromo-benzyl)-1H-pyrazole-3-carboxylic acid; 6-Methyl-3-(1H-tetrazol-5-yl)-4,6-dihydro-1H-furo[3,4-c]pyrazole; 3-(1H-Tetrazol-5-yl)-4,6-dihydro-1H-thieno[3,4-c]pyrazole; 3-(1H-Tetrazol-5-yl)-1,4-dihydro-cyclopentapyrazole; 3-(1H-Tetrazol-5-yl)-1,6-dihydro-cyclopentapyrazole; 3-(1H-Tetrazol-5-yl)-2,6-dihydro-4H-furo[3,4-c]pyrazole; 5-Ethyl-3-(1H-tetrazol-5-yl)-2,4,5,6-tetrahydro-cyclopentapyrazole; and 5-(5-Isopropyl-1H-pyrazol-3-yl)-1H-tetrazole; or a pharmaceutically acceptable salt thereof. In another embodiment, said composition further comprises at least one agent selected from the group consisting of α-glucosidase inhibitor, aldose reductase inhibitor, biguanide, HMG-CoA reductase inhibitor, squalene synthesis inhibitor, fibrate, LDL catabolism enhancer, angiotensin converting enzyme inhibitor, insulin secretion enhancer and thiazolidinedione.

As used herein “composition” means a material comprising at least one component. A pharmaceutical composition is an example of a composition. A pharmaceutical composition means a composition comprising at least one active ingredient, whereby the composition is amenable to investigation for a specified, efficacious outcome in a mammal (for example, a human). Those of ordinary skill in the art will understand and appreciate the techniques appropriate for determining whether an active ingredient has a desired efficacious outcome based upon the needs of the artisan.

Compositions described herein can include a pharmaceutically or physiologically acceptable carrier. Suitable pharmaceutically-acceptable carriers are available to those in the art; for example, see Remington: The Science and Practice or Pharmacy, 20^{th} Edition, 2000, Lippincott, Williams & Wilkons, (Gennaro et al., eds.). While it is possible that, for use in prophylaxis or treatment, a compound of the invention can in an alternative use be administered as a raw or pure chemical, it can also be desirable to present the compound or active ingredient as a pharmaceutical formulation or composition.

The invention thus further provides pharmaceutical formulations comprising a compound of the invention or a pharmaceutically acceptable salt or derivative thereof together with one or more pharmaceutically acceptable carriers thereof and/or prophylactic ingredients. The carrier(s) are “acceptable” in the sense of being compatible with the other ingredients of the formulation and not overly deleterious to the recipient thereof.

Pharmaceutical formulations include those suitable for oral, rectal, nasal, topical (including buccal and sub-lingual), vaginal or parenteral (including intramuscular, sub-cutaneous and intravenous) administration or in a form suitable for administration by inhalation or insufflation.

The compounds of the invention, together with a conventional adjuvant, carrier, or diluent, can be placed into the form of pharmaceutical formulations and unit dosages thereof, and in such form can be employed as solids, such as tablets or filled capsules, or liquids such as solutions, suspensions, emulsions, elixirs, gels or capsules filled with the same, all for oral use, in the form of suppositories for rectal administration; in the form of liquids, gels, lotions or ointments for topical use, or in the form of sterile injectable solutions for parenteral (including subcutaneous) use. Such pharmaceutical compositions and unit dosage forms thereof can comprise conventional ingredients in conventional proportions, with or without additional active compounds or principles, and such unit dosage forms can contain any suitable effective amount of the active ingredient commensurate with the intended daily dosage range to be employed.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances which can also act as diluents, flavouring agents, solubilizers, lubricants, suspending agents, binders, preservatives, tablet disintegrating agents, or an encapsulating material. In powders, the carrier can be a finely divided solid which is in a mixture with the finely divided active component. In tablets, the active component can be mixed with the carrier having the necessary binding capacity in suitable proportions and compacted to the desire shape and size.

Powders and tablets can contain varying percentage amounts of the active compound. A representative amount in a powder or tablet can contain from 0.5 to about 90 percent of the active compound; however, an artisan would know when amounts outside of this range are necessary. Suitable carriers for powders and tablets are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as an admixture of fatty acid glycerides or cocoa butter, can be first melted and the active component can be dispersed homogeneously therein, as by stirring. The molten homogenous mixture can then poured into convenient sized molds, allowed to cool, and thereby to solidify. Formulations suitable for vaginal administration can be presented as pessaries, tampons, creams, gels, pastes, foams or sprays containing in addition to the active ingredient such carriers as are known in the art to be appropriate.

Liquid form preparations include solutions, suspensions, and emulsions, for example, water or water-propylene glycol solutions. For example, parenteral injection liquid preparations can be formulated as solutions in aqueous polyethylene glycol solution. Injectable preparations, for example, sterile injectable aqueous or oleaginous suspensions can be formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol. Among the acceptable vehicles and solvents that can be employed are water, Ringer's solution, and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables.

The compositions according to the present invention can thus be formulated for parenteral administration (that is, by injection, for example, bolus injection or continuous infusion) and can be presented in unit dose form in ampoules, pre-filled syringes, small volume infusion or in multi-dose containers with an added preservative. The compositions can take such forms as suspensions, solutions, or emulsions in oily or aqueous vehicles, and can contain formulatory agents such as suspending, stabilizing and/or dispersing agents. Alternatively, the active ingredient can be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavors, stabilizing and thickening agents, as desired. Aqueous suspensions suitable for oral use can be made by dispersing the finely divided active component in water with viscous material, such as natural or synthetic gums, resins, methylcellulose, sodium carboxymethylcellulose, or other well known suspending agents. Also included are solid form preparations which are intended to be converted, shortly before use, to liquid form preparations for oral administration. Such liquid forms include solutions, suspensions, and emulsions. These preparations can contain, in addition to the active component, colorants, flavors, stabilizers, buffers, artificial and natural sweeteners, dispersants, thickeners, solubilizing agents, and the like.

For topical administration to the epidermis the compositions according to the invention can be formulated as ointments, creams or lotions, or as a transdermal patch. Ointments and creams can, for example, be formulated with an aqueous or oily base with the addition of suitable thickening and/or gelling agents. Lotions can be formulated with an aqueous or oily base and can, in general, also contain one or more emulsifying agents, stabilizing agents, dispersing agents, suspending agents, thickening agents, or coloring agents.

Formulations suitable for topical administration in the mouth include lozenges comprising active agent in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions can be applied directly to the nasal cavity by conventional means, for example with a dropper, pipette or spray. The formulations can be provided in single or multi-dose form. In the latter case of a dropper or pipette, this can be achieved by the individual administering an appropriate, predetermined volume of the solution or suspension. In the case of a spray, this can be achieved for example by means of a metering atomizing spray pump. Administration to the respiratory tract can also be achieved by means of an aerosol formulation in which the active ingredient is provided in a pressurized pack with a suitable propellant. If a pharmaceutical composition is administered as an aerosol, for example a nasal aerosols or by inhalation, this can be carried out, for example, using a spray, a nebulizer, a pump nebulizer, an inhalation apparatus, a metered inhaler or a dry powder inhaler. Pharmaceutical forms for administration of the compositions of the invention as an aerosol can be prepared by processes well-known to the person skilled in the art. For their preparation, for example, solutions or dispersions of the compounds of the invention in water, water/alcohol mixtures or suitable saline solutions can be employed using customary additives, for example benzyl alcohol or other suitable preservatives, absorption enhancers for increasing the bioavailability, solubilizers, dispersants and others, and, if appropriate, customary propellants, for example include carbon dioxide, CFC's, such as, dichlorodifluoromethane, trichlorofluoromethane, or dichlorotetrafluoroethane; and the like. The aerosol can conveniently also contain a surfactant such as lecithin. The dose of drug can be controlled by provision of a metered valve.

In formulations intended for administration to the respiratory tract, including intranasal formulations, the compound will generally have a small particle size, for example, of the order of 10 microns or less. Such a particle size can be obtained by means known in the art, for example by micronization. When desired, formulations adapted to give sustained release of the active ingredient can be employed.

Alternatively the active ingredients can be provided in the form of a dry powder, for example, a powder mix of the compound in a suitable powder base such as lactose, starch, starch derivatives such as hydroxypropylmethyl cellulose and polyvinylpyrrolidone (PVP). Conveniently the powder carrier can form a gel in the nasal cavity. The powder composition can be presented in unit dose form, for example, in capsules or cartridges of, for example, gelatin, or blister packs from which the powder may be administered by means of an inhaler.

In addition to the formulations described previously, the compounds can also be formulated as a depot preparation. Such long acting formulations can be administered by implantation (for example, subcutaneously or intramuscularly) or by intramuscular injection. Thus, for example, the compounds can be formulated with suitable polymeric or hydrophobic materials (for example, as an emulsion in an acceptable oil) or ion exchange resins, or as sparingly soluble derivatives, for example, as a sparingly soluble salt. In addition, a composition can be delivered via a controlled release system such as a pump.

Additionally, the compositions can be delivered using a sustained-release system, such as semipermeable matrices of solid hydrophobic polymers containing the therapeutic agent. Various sustained release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for modulator stabilization can be employed.

The present invention provides kits for use by a consumer to prevent or treat a lipid-associated disorder. A kit can comprise a pharmaceutical composition of the invention and instructions describing a method of using the pharmaceutical composition to prevent or treat a lipid-associated disorder. For example, a kit can contain at least one dosage unit of a niacin receptor partial agonist and at least one dosage unit of niacin or a niacin analog. In addition, a kit can include other therapeutic agents used in combination with the compositions of the invention.

The invention provides a kit for preventing or treating a lipid-associated disorder comprising at least one dosage unit of a niacin receptor partial agonist and at least one dosage unit of niacin or a niacin analog, wherein said niacin receptor partial agonist is present in an amount effective to reduce flushing induced by niacin or a niacin analog in said subject and wherein said niacin or niacin analog is present in a lipid altering amount. In one embodiment, said kit comprises a dosage unit of niacin and in another embodiment, said kit comprises a dosage unit of a niacin analog. In one embodiment, said niacin analog is a structural analog of niacin and in another embodiment, said niacin analog is a functional analog of niacin. In a further embodiment, flushing is completely reduced or eliminated. In a yet further embodiment, said dosage unit of niacin or a niacin analog is at least 500 mg per day. In one embodiment, said niacin receptor partial agonist comprises a compound of Formula (I):

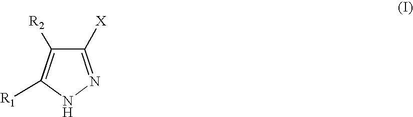

or a pharmaceutically acceptable salt thereof, wherein: X is a carboxyl or a tetrazol-5-yl group; R₁ is iso-propyl, 3-fluoro-benzyl, 3-chloro-benzyl, or 3-bromo-benzyl; and R₂ is H; or R₁ and R₂ together with the two pyrazole ring carbons to which they are bonded form a 5-membered carbocyclic ring optionally substituted with ethyl or a 5-membered heterocyclic ring optionally substituted with methyl. For example, said niacin receptor partial agonist can comprise a compound selected from the group consisting of: 3-(1H-Tetrazol-5-yl)-1,4,5,6-tetrahydro-cyclopentapyrazole; 5-(3-Fluoro-benzyl)-1H-pyrazole-3-carboxylic acid; 5-(3-Chloro-benzyl)-1H-pyrazole-3-carboxylic acid; 5-(3-Bromo-benzyl)-1H-pyrazole-3-carboxylic acid; 6-Methyl-3-(1H-tetrazol-5-yl)-4,6-dihydro-1H-furo[3,4-c]pyrazole; 3-(1H-Tetrazol-5-yl)-4,6-dihydro-1H-thieno[3,4-c]pyrazole; 3-(1H-Tetrazol-5-yl)-1,4-dihydro-cyclopentapyrazole; 3-(1H-Tetrazol-5-yl)-1,6-dihydro-cyclopentapyrazole; 3-(1H-Tetrazol-5-yl)-2,6-dihydro-4H-furo[3,4-c]pyrazole; 5-Ethyl-3-(1H-tetrazol-5-yl)-2,4,5,6-tetrahydro-cyclopentapyrazole; and 5-(5-Isopropyl-1H-pyrazol-3-yl)-1H-tetrazole; or a pharmaceutically acceptable salt thereof. In another embodiment, said kit further comprises at least one agent selected from the group consisting of α-glucosidase inhibitor, aldose reductase inhibitor, biguanide, HMG-CoA reductase inhibitor, squalene synthesis inhibitor, fibrate, LDL catabolism enhancer, angiotensin converting enzyme inhibitor, insulin secretion enhancer and thiazolidinedione.

The compositions of the invention can be administrated in a wide variety of oral, topical or parenteral dosage forms. It will be obvious to those skilled in the art that the dosage forms can comprise, as the active component, either a compound of the invention or a pharmaceutically acceptable salt of a compound of the invention.

The dosage of active ingredient, or an active salt or derivative thereof, required for use in prophylaxis or treatment will vary not only with the particular salt selected but also with the route of administration, the nature of the condition being treated and the age and condition of the individual and will ultimately be at the discretion of the attendant physician or clinician. In general, one skilled in the art understands how to extrapolate in vivo data obtained in a model system, typically an animal model, to another, such as a human. An illustrative but not intended to be limiting in vivo animal model is provided as an Example infra. In some circumstances, these extrapolations can merely be based on the weight of the animal model in comparison to another, such as a mammal, preferably a human, however, more often, these extrapolations are not simply based on weights, but rather incorporate a variety of factors. Representative factors include the type, age, weight, sex, diet and medical condition of the individual, the severity of the disease, the route of administration, pharmacological considerations such as the activity, efficacy, pharmacokinetic and toxicology profiles of the particular compound employed, whether a drug delivery system is utilized, on whether an acute or chronic disease state is being treated or prophylaxis is conducted or on whether combination therapy is used. The dosage regimen for preventing or treating a disease condition with the compounds and/or compositions of this invention is selected in accordance with a variety factors as cited above. Thus, the actual dosage regimen employed can vary widely and therefore can deviate from a preferred dosage regimen and one skilled in the art will recognize that dosage and dosage regimen outside these typical ranges can be tested and, where appropriate, can be used in the methods of this invention.

The desired dose can conveniently be presented in a single dose or as divided doses administered at appropriate intervals, for example, as two, three, four or more sub-doses per day. The sub-dose itself may be further divided, for example, into a number of discrete loosely spaced administrations. The daily dose can be divided, especially when relatively large amounts are administered as deemed appropriate, into several, for example 2, 3 or 4, part administrations. If appropriate, depending on individual behavior, it can be necessary to deviate upward or downward from the daily dose indicated.

A kit as used in the instant application includes a container for containing a pharmaceutical composition of the invention and can also include divided containers such as a divided bottle or a divided foil packet. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Generally, the strength of the sheet is such that the tablets or capsules can be removed from the blister pack by manually applying pressure on the recesses whereby an opening is formed in the sheet at the place of the recess. The tablet or capsule can then be removed via said opening.

It can be desirable to provide a written memory aid, where the written memory aid is of the type containing information and/or instructions for the physician, pharmacist or subject, for example, in the form of numbers next to the tablets or capsules whereby the numbers correspond with the days of the regimen which the tablets or capsules so specified should be ingested or a card which contains the same type of information. Another example of such a memory aid is a calendar printed on the card for example, as follows “First Week, Monday, Tuesday,” . . . etc. . . . “Second Week, Monday, Tuesday” etc. Other variations of memory aids will be readily apparent.

Another specific embodiment of a kit is a dispenser designed to dispense the daily doses one at a time. The dispenser can be equipped with a memory-aid, so as to further facilitate compliance with the regimen. An example of such a memory-aid is a mechanical counter which indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

The invention further provides a kit for preventing or treating a lipid-associated disorder comprising at least one dosage unit of a niacin receptor partial agonist and at least one separate dosage unit of niacin or a niacin analog, wherein said niacin receptor partial agonist is present in an amount effective to reduce flushing induced by niacin or a niacin analog in said subject and wherein said niacin or niacin analog is present in a lipid altering amount. In one embodiment, said kit comprises a dosage unit of niacin and in another embodiment, said kit comprises a dosage unit of a niacin analog. In one embodiment, said niacin analog is a structural analog of niacin and in another embodiment, said niacin analog is a functional analog of niacin. In a further embodiment, flushing is completely reduced or eliminated. In a yet further embodiment, said dosage unit of niacin or a niacin analog is at least 500 mg per day. In one embodiment, said niacin receptor partial agonist comprises a compound of Formula (I):

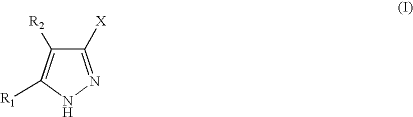

or a pharmaceutically acceptable salt thereof, wherein: X is a carboxyl or a tetrazol-5-yl group; R₁ is iso-propyl, 3-fluoro-benzyl, 3-chloro-benzyl, or 3-bromo-benzyl; and R₂ is H; or R₁ and R₂ together with the two pyrazole ring carbons to which they are bonded form a 5-membered carbocyclic ring optionally substituted with ethyl or a 5-membered heterocyclic ring optionally substituted with methyl. For example, said niacin receptor partial agonist can comprise a compound selected from the group consisting of: 3-(1H-Tetrazol-5-yl)-1,4,5,6-tetrahydro-cyclopentapyrazole; 5-(3-Fluoro-benzyl)-1H-pyrazole-3-carboxylic acid; 5-(3-Chloro-benzyl)-1H-pyrazole-3-carboxylic acid; 5-(3-Bromo-benzyl)-1H-pyrazole-3-carboxylic acid; 6-Methyl-3-(1H-tetrazol-5-yl)-4,6-dihydro-1H-furo[3,4-c]pyrazole; 3-(1H-Tetrazol-5-yl)-4,6-dihydro-1H-thieno[3,4-c]pyrazole; 3-(1H-Tetrazol-5-yl)-1,4-dihydro-cyclopentapyrazole; 3-(1H-Tetrazol-5-yl)-1,6-dihydro-cyclopentapyrazole; 3-(1H-Tetrazol-5-yl)-2,6-dihydro-4H-furo[3,4-c]pyrazole; 5-Ethyl-3-(1H-tetrazol-5-yl)-2,4,5,6-tetrahydro-cyclopentapyrazole; and 5-(5-Isopropyl-1H-pyrazol-3-yl)-1H-tetrazole; or a pharmaceutically acceptable salt thereof. In another embodiment, said kit further comprises at least one agent selected from the group consisting of α-glucosidase inhibitor, aldose reductase inhibitor, biguanide, HMG-CoA reductase inhibitor, squalene synthesis inhibitor, fibrate, LDL catabolism enhancer, angiotensin converting enzyme inhibitor, insulin secretion enhancer and thiazolidinedione.

In addition, the invention provides a kit for preventing or treating a lipid-associated disorder comprising at least one pre-dosage unit of a niacin receptor partial agonist and at least one separate dosage unit of niacin or a niacin analog, wherein said niacin receptor partial agonist is present in an amount effective to reduce flushing induced by niacin or a niacin analog in said subject and wherein said niacin or niacin analog is present in a lipid altering amount. A pre-dosage unit is a dose of a niacin receptor partial agonist which is intended to be administered prior to some other dosage unit. In one embodiment, said kit comprises a dosage unit of niacin and in another embodiment, said kit comprises a dosage unit of a niacin analog. In one embodiment, said niacin analog is a structural analog of niacin and in another embodiment, said niacin analog is a functional analog of niacin. In a further embodiment, flushing is completely reduced or eliminated. In a yet further embodiment, said dosage unit of niacin or a niacin analog is at least 500 mg per day. In one embodiment, said niacin receptor partial agonist comprises a compound of Formula (I):

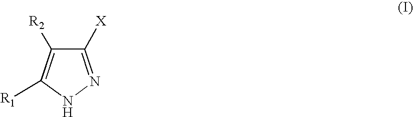

or a pharmaceutically acceptable salt thereof, wherein: X is a carboxyl or a tetrazol-5-yl group; R₁ is iso-propyl, 3-fluoro-benzyl, 3-chloro-benzyl, or 3-bromo-benzyl; and R₂ is H; or R₁ and R₂ together with the two pyrazole ring carbons to which they are bonded form a 5-membered carbocyclic ring optionally substituted with ethyl or a 5-membered heterocyclic ring optionally substituted with methyl. For example, said niacin receptor partial agonist can comprise a compound selected from the group consisting of: 3-(1H-Tetrazol-5-yl)-1,4,5,6-tetrahydro-cyclopentapyrazole; 5-(3-Fluoro-benzyl)-1H-pyrazole-3-carboxylic acid; 5-(3-Chloro-benzyl)-1H-pyrazole-3-carboxylic acid; 5-(3-Bromo-benzyl)-1H-pyrazole-3-carboxylic acid; 6-Methyl-3-(1H-tetrazol-5-yl)-4,6-dihydro-1H-furo[3,4-c]pyrazole; 3-(1H-Tetrazol-5-yl)-4,6-dihydro-1H-thieno[3,4-c]pyrazole; 3-(1H-Tetrazol-5-yl)-1,4-dihydro-cyclopentapyrazole; 3-(1H-Tetrazol-5-yl)-1,6-dihydro-cyclopentapyrazole; 3-(1H-Tetrazol-5-yl)-2,6-dihydro-4H-furo[3,4-c]pyrazole; 5-Ethyl-3-(1H-tetrazol-5-yl)-2,4,5,6-tetrahydro-cyclopentapyrazole; and 5-(5-Isopropyl-1H-pyrazol-3-yl)-1H-tetrazole; or a pharmaceutically acceptable salt thereof. In another embodiment, said kit further comprises at least one agent selected from the group consisting of α-glucosidase inhibitor, aldose reductase inhibitor, biguanide, HMG-CoA reductase inhibitor, squalene synthesis inhibitor, fibrate, LDL catabolism enhancer, angiotensin converting enzyme inhibitor, insulin secretion enhancer and thiazolidinedione.

General terms have been used herein such as “for example” and “comprising,” and are defined herein according to their standard meaning. The terms “for example” and “such as” are intended to exemplify without limitation.

One aspect of the present invention pertains to a niacin receptor partial agonist and niacin or a niacin analog, as described herein, for use in a method of treatment of the human or animal body by therapy.

Another aspect of the present invention pertains to a niacin receptor partial agonist, as described herein, for use in a method of treatment of flushing induced by niacin or a niacin analog, of the human or animal body by therapy. Another aspect of the present invention pertains to a method for the treatment of flushing induced by niacin or a niacin analog comprising administering to a subject suffering from said condition a therapeutically-effective amount of a niacin receptor partial agonist, as described herein, preferably in the form of a pharmaceutical composition.

One aspect of the present invention pertains to a method for the treatment of a lipid-associated disorder comprising administering to a subject suffering from said condition a therapeutically-effective amount of a niacin receptor partial agonist and niacin or a niacin analog, as described herein, preferably in the form of a pharmaceutical composition. Another aspect of the present invention pertains to a niacin receptor partial agonist and niacin or a niacin analog, as described herein, for use in a method of treatment of a lipid-associated disorder of the human or animal body by therapy.

One aspect of the present invention pertains to use of a niacin receptor partial agonist and niacin or a niacin analog, as described herein, for the manufacture of a medicament for use in the treatment of flushing induced by and niacin or a niacin analog. Another aspect of the present invention pertains to use of a niacin receptor partial agonist, as described herein, for the manufacture of a medicament for use in the treatment of flushing induced by and niacin or a niacin analog. In addition, one aspect of the present invention pertains to use of a niacin receptor partial agonist and niacin or a niacin analog, as described herein, for the manufacture of a medicament for use in the treatment of a lipid-associated disorder.

### EXAMPLES

The following Examples are provided for illustrative purposes and not as a means of limitation. One of ordinary skill in the art would be able to design equivalent assays and methods based on the disclosure herein, all of which form part of the present invention.

### Example 1

### Niacin Receptor Partial Agonists Block Flushing Induced by Niacin

This example shows that niacin receptor partial agonists can block flushing induced by niacin. Several niacin receptor partial agonists from Table A were tested for ability to block niacin-induced flushing in mice. Flushing was measured using a Laser Dopler.

In these experiments, the control group contained anesthetized mice that were administered niacin alone and flushing above baseline was measured over time. The experimental group contained anesthetized mice that were administered a niacin receptor partial agonist about 10 minutes before administration of niacin. Flushing above baseline after niacin administration was then measured over time and compared to mice treated with niacin alone.

### Representative Data:

Control mice treated with niacin alone began to flush after 1.5 minutes with flush peaking at about 150% of baseline at 3 minutes and returning to about 30% of baseline within about 15 minutes

Compound 2: Mice treated with niacin alone began to flush after 1.5 minutes with flush peaking at about 100% to 150% of baseline at 3 minutes and returning to about 30% to 45% of baseline within about 15 minutes. Treatment of mice with Compound 2 prior to niacin administration resulted in 0% change from baseline at 3 minutes with change from baseline slowly increasing to about 15% above baseline within 15 minutes. Treatment of mice with Compound 3 prior to niacin administration resulted in about 18% change from baseline at 3 minutes with change from baseline slowly decreasing to about 13% above baseline at 15 minutes. Treatment of mice with Compound 4 prior to niacin administration resulted in 15% change from baseline at 3 minutes with change from baseline slowly increasing to about 30% above baseline within 15 minutes.

### Example 2

### Mice Treated with Niacin Receptor Partial Agonists Retain the Ability to Flush in Response to PGD2 Administration

As shown in Example 1, treatment of mice with a niacin receptor partial agonist blocks flushing induced by niacin. This example shows that mice treated with a niacin receptor partial agonist retain the ability to flush when given a PGD2, a known flush-inducing agent.

In this experiment mice were treated with Compound 1 about 10 minutes prior to niacin administration and the experiment was performed as in Example 1. After re-establishment of baseline, PGD2 was administered and flushing was recorded. Specifically, in this experiment mice treated with niacin alone began to flush after 1.5 minutes with flush peaking at about 60% of baseline at 3 minutes and returning to about 20% of baseline within about 15 minutes. Treatment of mice with Compound 1 about 10 minutes prior to niacin administration resulted in 10% change from baseline at 3 minutes with change from baseline slowly increasing to about 20% above baseline within 15 minutes. Baseline was then re-established at 20% above baseline and PGD2 was administered. Flushing began about 1.5 minutes later and peaked about 6 minutes after PGD2 administration at about 70% of the original baseline. This experiment shows that the ability of mice to flush when given PGD2 was not reduced by the niacin receptor partial agonist, while flushing induced by niacin was significantly reduced.

### Example 3

### NEFA Competition

This example shows that a niacin receptor partial agonist, Compound 1, does not interfere with the reduction in free fatty acids induced by niacin.

Mice were given either: vehicle, vehicle plus niacin, or Compound 1 plus niacin. After 10 minutes the mice were euthanized and blood was collected. The blood samples were processed and tested for free fatty acid release using the non-esterified fatty-acid (NEFA) assay (the NEFA-C assay kit from Waco Chemicals USA, Richmond, Va.). The NEFA assay was done as per manufacturer suggested protocol. The concentration of free fatty acid measured for the vehicle sample was 0.9 mM, vehicle plus niacin was 0.4 mM, and Compound 1 plus niacin was 0.38 mM. Therefore, the niacin receptor partial agonist did not interfere with the reduction in free fatty acids induced by niacin.

### Example 4

### Measurement of Free Fatty Acid Levels in Rats and Lipolysis in Human Adipocytes

This example shows that free fatty acid levels can be measured in rats. This example also shows that free fatty acid levels can be measured in human adipocytes.

### Rat Assay

Catheters are surgically implanted into the jugular veins of male Sprague Dawley rats. Rats are given a few days to recover from catheter implantation surgery and then the following day rats are deprived of food and approximately 16 hours later are given interperitoneal (IP) injections of either vehicle, or niacin [NA] at 15 mg/kg, 30 mg/kg or 45 mg/kg body weight. A niacin analog can be tested in the same manner. Blood is drawn (˜200 ml) at various time points and plasma is isolated following centrifugation. Plasma FFAs are then measured via the NEFA C kit according to manufacturer specifications (Wako Chemicals USA, Inc).

### Human Adipocyte Lipolysis Assay:

Adipocytes are obtained from ZenBio (Research Triangle, North Carolina) and the lipolysis assay is performed according to manufacturer's protocol. An elevation of intracellular cAMP levels and concomitant activation of lipolysis via hormone sensitive lipase is accomplished using isoproterenol at concentrations and times determined empirically. Lipolysis is allowed to continue for the desired time in the presence or absence of a compound of interest (for example, niacin or a niacin analog). At least five compound concentrations are tested allowing for non-linear regression analysis and determination of an EC₅₀ value. The percent of glycerol production is measured calorimetrically and is compared to standards (ZenBio).

### Example 5

### Mouse Atherosclerosis Model

Adiponectin-deficient mice generated through knocking out the adiponectin gene have been shown to be predisposed to atherosclerosis and to be insulin resistant. The mice are also a suitable model for ischemic heart disease [Matsuda, M et al. J Biol Chem (2002) July, and references cited therein, the disclosures of which are incorporated herein by reference in their entirety].

Adiponectin knockout mice are housed (7-9 mice/cage) under standard laboratory conditions at 22° C. and 50% relative humidity. The mice are dosed by micro-osmotic pumps, inserted using isoflurane anesthesia, to provide compounds of the invention, saline, or an irrelevant compound to the mice subcutaneously (s.c.). Neointimal thickening and ischemic heart disease are determined for different groups of mice sacrificed at different time intervals. Significant differences between groups (comparing compounds of the invention to saline-treated) are evaluated using Student t-test.

The foregoing mouse model of atherosclerosis is provided by way of illustration and not limitation. By way of further example, Apolipoprotein E-deficient mice have also been shown to be predisposed to atherosclerosis [Plump A S et al., Cell (1992) 71:343-353; the disclosure of which is hereby incorporated by reference in its entirety].

Another model that can be used is that of diet-induced atherosclerosis in C57BL/6J mice, an inbred strain known to be susceptible to diet-induced atherosclerotic lesion formation. This model is well known to persons of ordinary skill in the art [Kamada N et al., J Atheroscler Thromb (2001) 8:1-6; Garber D W et al., J Lipid Res (2001) 42:545-52; Smith J D et al., J Intern Med (1997) 242:99-109; the disclosure of each of which is hereby incorporated by reference in its entirety].

### Example 6

### In Vivo Pig Model of HDL-Cholesterol and Atherosclerosis

The utility of a compound of the present invention as a medical agent in the prevention or treatment of a lipid-associated disorder is demonstrated, for example, by the activity of the compound in lowering the ratio of total cholesterol to HDL-cholesterol, in elevating HDL-cholesterol, or in protection from atherosclerosis in an in vivo pig model. Pigs are used as an animal model because they reflect human physiology, especially lipid metabolism, more closely than most other animal models. An illustrative in vivo pig model not intended to be limiting is presented here.

Yorkshire albino pigs (body weight 25.5±4 kg) are fed a saturated fatty acid rich and cholesterol rich (SFA-CHO) diet during 50 days (1 kg chow 35 kg-1 pig weight), composed of standard chow supplemented with 2% cholesterol and 20% beef tallow [Royo T et al., European Journal of Clinical Investigation (2000) 30:843-52; which disclosure is hereby incorporated by reference in its entirety]. Saturated to unsaturated fatty acid ratio is modified from 0.6 in normal pig chow to 1.12 in the SFA-CHO diet. Animals are divided into two groups, one group (n=8) fed with the SFA-CHO diet and treated with placebo and one group (n=8) fed with the SFA-CHO diet and treated with the modulator (3.0 mg kg-1). Control animals are fed a standard chow for a period of 50 days. Blood samples are collected at baseline (2 days after the reception of the animals), and 50 days after the initiation of the diet. Blood lipids are analyzed. The animals are sacrificed and necropsied.

Alternatively, the foregoing analysis comprises a plurality of groups each treated with a different dose of the compound of interest. Doses include, for example: 0.1 mg kg-1, 0.3 mg kg-1, 1.0 mg kg-1, 3.0 mg kg-1, 10 mg kg-1, 30 mg kg-1 and 100 mg kg-1. Alternatively, the foregoing analysis is carried out at a plurality of timepoints, for example, 10 weeks, 20 weeks, 30 weeks, 40 weeks, and 50 weeks.

### HDL-Cholesterol

Blood is collected in trisodium citrate (3.8%, 1:10). Plasma is obtained after centrifugation (1200 g 15 mm) and immediately processed. Total cholesterol, HDL-cholesterol, and LDL-cholesterol are measured using the automatic analyzer Kodak Ektachem DT System (Eastman Kodak Company, Rochester, N.Y., USA). Samples with value parameters above the range are diluted with the solution supplied by the manufacturer and then re-analyzed. The total cholesterol/HDL-cholesterol ratio is determined. Comparison is made of the level of HDL-cholesterol between groups. Comparison is made of the total cholesterol/HDL-cholesterol ratio between groups.

Elevation of HDL-cholesterol or reduction of the total cholesterol/HDL-cholesterol ratio on administration of the compound of interest is taken as indicative of the compound having the aforesaid utility.

### Atherosclerosis

The thoracic and abdominal aortas are removed intact, opened longitudinally along the ventral surface, and fixed in neutral-buffered formalin after excision of samples from standard sites in the thoracic and abdominal aorta for histological examination and lipid composition and synthesis studies. After fixation, the whole aortas are stained with Sudan IV and pinned out flat, and digital images are obtained with a TV camera connected to a computerized image analysis system (Image Pro Plus; Media Cybernetics, Silver Spring, Md.) to determine the percentage of aortic surface involved with atherosclerotic lesions [Gerrity R G et al, Diabetes (2001) 50:1654-65; Cornhill J F et al, Arteriosclerosis, Thrombosis, and Vascular Biology (1985) 5:415-26; which disclosures are hereby incorporated by reference in their entirety]. Comparison is made between groups of the percentage of aortic surface involved with atherosclerotic lesions.

Reduction of the percentage of aortic surface involved with atherosclerotic lesions on administration of the compound of interest is taken as indicative of the compound having the aforesaid utility.

### Plasma Free Fatty Acids

It would be readily apparent to anyone of ordinary skill in the art that the foregoing in vivo pig model is easily modified in order to address, without limitation, the activity of the compound in lowering plasma free fatty acids.

### Example 7

### Assays for Determination of GPCR Activation

A variety of approaches are available for assessment of activation of human GPCRs. The following are illustrative; those of ordinary skill in the art are credited with the ability to determine those techniques that are preferentially beneficial for the needs of the artisan.

1. Membrane Binding Assays: [³⁵S]GTPγS Assay

When a G protein-coupled receptor is in its active state, either as a result of ligand binding or constitutive activation, the receptor couples to a G protein and stimulates the release of GDP and subsequent binding of GTP to the G protein. The alpha subunit of the G protein-receptor complex acts as a GTPase and slowly hydrolyzes the GTP to GDP, at which point the receptor normally is deactivated. Activated receptors continue to exchange GDP for GTP. The non-hydrolyzable GTP analog, [³⁵S]GTPγS, can be utilized to demonstrate enhanced binding of [³⁵S]GTPγS to membranes expressing activated receptors. The advantage of using [³⁵S]GTPγS binding to measure activation is that: (a) it is generically applicable to all G protein-coupled receptors; (b) it is proximal at the membrane surface making it less likely to pick-up molecules which affect the intracellular cascade.

The assay utilizes the ability of G protein coupled receptors to stimulate [³⁵S]GTPγS binding to membranes expressing the relevant receptors. The assay can, therefore, be used in the direct identification method to screen candidate compounds to endogenous GPCRs and non-endogenous, constitutively activated GPCRs. The assay is generic and has application to drug discovery at all G protein-coupled receptors.

The [³⁵S]GTPγS assay is incubated in 20 mM HEPES and between 1 and about 20 mM MgCl₂ (this amount can be adjusted for optimization of results, although 20 mM is preferred) pH 7.4, binding buffer with between about 0.3 and about 1.2 nM [³⁵S]GTPγS (this amount can be adjusted for optimization of results, although 1.2 is preferred) and 12.5 to 75 μg membrane protein (e.g, 293 cells expressing the GPR35; this amount can be adjusted for optimization) and 10 μM GDP (this amount can be changed for optimization) for 1 hour. Wheatgerm agglutinin beads (25 μl; Amersham) are then added and the mixture incubated for another 30 minutes at room temperature. The tubes are then centrifuged at 1500×g for 5 minutes at room temperature and then counted in a scintillation counter.

2. Adenylyl Cyclase

A Flash Plate™ Adenylyl Cyclase kit (New England Nuclear; Cat. No. SMP004A) designed for cell-based assays can be modified for use with crude plasma membranes. The Flash Plate wells can contain a scintillant coating which also contains a specific antibody recognizing cAMP. The cAMP generated in the wells can be quantitated by a direct competition for binding of radioactive cAMP tracer to the cAMP antibody. The following serves as a brief protocol for the measurement of changes in cAMP levels in whole cells that express a receptor.

Transfected cells are harvested approximately twenty four hours after transient transfection. Media is carefully aspirated off and discarded. 10 ml of PBS is gently added to each dish of cells followed by careful aspiration. 1 ml of Sigma cell dissociation buffer and 3 ml of PBS are added to each plate. Cells are pipetted off the plate and the cell suspension is collected into a 50 ml conical centrifuge tube. Cells are then centrifuged at room temperature at 1,100 rpm for 5 minutes. The cell pellet is carefully re-suspended into an appropriate volume of PBS (about 3 ml/plate). The cells are then counted using a hemocytometer and additional PBS is added to give the appropriate number of cells (with a final volume of about 50 μl/well).

cAMP standards and Detection Buffer (comprising 1 μCi of tracer [¹²⁵I] cAMP (50 μl) to 11 ml Detection Buffer) is prepared and maintained in accordance with the manufacturer's instructions. Assay Buffer is prepared fresh for screening and contains 50 μl of Stimulation Buffer, 3 μl of candidate compound (12 μM final assay concentration) and 50 μl cells. Assay Buffer is stored on ice until utilized. The assay, preferably carried out, for example, in a 96-well plate, is initiated by addition of 50 μl of cAMP standards to appropriate wells followed by addition of 50 μl of PBSA to wells H11 and H12. 50 μl of Stimulation Buffer is added to all wells. DMSO (or selected candidate compounds) is added to appropriate wells using a pin tool capable of dispensing 3 μl of compound solution, with a final assay concentration of 12 μM candidate compound and 100 μl total assay volume. The cells are then added to the wells and incubated for 60 minutes at room temperature. 100 μl of Detection Mix containing tracer cAMP is then added to the wells. Plates are then incubated additional 2 hours followed by counting in a Wallac MicroBeta scintillation counter. Values of cAMP/well are then extrapolated from a standard cAMP curve which is contained within each assay plate.

3. Cell-Based cAMP for Gi Coupled Target GPCRs

TSHR is a Gs coupled GPCR that causes the accumulation of cAMP upon activation. TSHR can be constitutively activated by mutating amino acid residue 623 (i.e., changing an alanine residue to an isoleucine residue). A Gi coupled receptor is expected to inhibit adenylyl cyclase, and, therefore, decrease the level of cAMP production, which can make assessment of cAMP levels challenging. An effective technique for measuring the decrease in production of cAMP as an indication of activation of a Gi coupled receptor can be accomplished by co-transfecting, non-endogenous, constitutively activated TSHR (TSHR-A623I) (or an endogenous, constitutively active Gs coupled receptor) as a “signal enhancer” with a Gi linked target GPCR to establish a baseline level of cAMP. Upon creating an endogenous or non-endogenous version of the Gi coupled receptor, the target GPCR is then co-transfected with the signal enhancer, and it is this material that can be used for screening. In some embodiments, this approach is preferably used in the direct identification of candidate compounds against Gi coupled receptors. It is noted that for a Gi coupled GPCR, when this approach is used, an inverse agonist of the target GPCR will increase the cAMP signal and an agonist will decrease the cAMP signal.

On day one, 2×10⁴ 293 cells/well are plated out. On day two, two reaction tubes are prepared (the proportions to follow for each tube are per plate): tube A is prepared by mixing 2 μg DNA of each receptor transfected into the mammalian cells, for a total of 4 μg DNA (e.g., pCMV vector; pCMV vector with mutated THSR (TSHR-A623I); TSHR-A623I and GPCR, etc.) in 1.2 ml serum free DMEM (Irvine Scientific, Irvine, Calif.); tube B is prepared by mixing 120 μl lipofectamine (Gibco BRL) in 1.2 ml serum free DMEM. Tubes A and B are then admixed by inversions (several times), followed by incubation at room temperature for 30-45 minutes. The admixture is referred to as the “transfection mixture”. Plated 293 cells are washed with 1×PBS, followed by addition of 10 ml serum free DMEM. 2.4 ml of the transfection mixture is then added to the cells, followed by incubation for 4 hours at 37° C./5% CO₂. The transfection mixture is then removed by aspiration, followed by the addition of 25 ml of DMEM/10% Fetal Bovine Serum. Cells are then incubated at 37° C./5% CO₂. After 24 hours incubation, cells are harvested and utilized for analysis.

A Flash Plate™ Adenylyl Cyclase kit (New England Nuclear; Cat. No. SMP004A) is designed for cell-based assays, but can be modified for use with crude plasma membranes depending on the need of the skilled artisan. The Flash Plate wells contain a scintillant coating which also contains a specific antibody recognizing cAMP. The cAMP generated in the wells can be quantitated by a direct competition for binding of radioactive cAMP tracer to the cAMP antibody. The following serves as a brief protocol for the measurement of changes in cAMP levels in whole cells that express a receptor of interest.

Transfected cells are harvested approximately twenty four hours after transient transfection. Media is carefully aspirated off and discarded. 10 ml of PBS is gently added to each dish of cells followed by careful aspiration. 1 ml of Sigma cell dissociation buffer and 3 ml of PBS is added to each plate. Cells are pipetted off the plate and the cell suspension is collected into a 50 ml conical centrifuge tube. Cells are then centrifuged at room temperature at 1,100 rpm for 5 minutes. The cell pellet is carefully re-suspended into an appropriate volume of PBS (about 3 ml/plate). The cells are then counted using a hemocytometer and additional PBS is added to give the appropriate number of cells (with a final volume of about 50 μl/well).

cAMP standards and Detection Buffer (comprising 1 μCi of tracer [125I] cAMP (50 μl) to 11 ml Detection Buffer) is prepared and maintained in accordance with the manufacturer's instructions. Assay Buffer should be prepared fresh for screening and contain 50 μl of Stimulation Buffer, 3 μl of candidate compound (12 μM final assay concentration) and 50 μl cells. Assay Buffer can be stored on ice until utilized. The assay can be initiated by addition of 50 μl of cAMP standards to appropriate wells followed by addition of 50 μl of PBSA to wells H-11 and H12. Fifty μl of Stimulation Buffer is added to all wells. Selected compounds (e.g., TSH) are added to appropriate wells using a pin tool capable of dispensing 3 μl of compound solution, with a final assay concentration of 12 μM candidate compound and 100 μl total assay volume. The cells are then added to the wells and incubated for 60 minutes at room temperature. 100 μl of Detection Mix containing tracer cAMP is then added to the wells. Plates are then incubated additional 2 hours followed by counting in a Wallac MicroBeta scintillation counter. Values of cAMP/well are extrapolated from a standard cAMP curve which is contained within each assay plate.

4. Reporter-Based Assays

a. CRE-LUC Reporter Assay (Gs-Associated Receptors)

293 or 293T cells are plated-out on 96 well plates at a density of 2×10⁴ cells per well and are transfected using Lipofectamine Reagent (BRL) the following day according to manufacturer instructions. A DNA/lipid mixture is prepared for each 6-well transfection as follows: 260 ng of plasmid DNA in 100 μl of DMEM is gently mixed with 2 μl of lipid in 100 μl of DMEM (the 260 ng of plasmid DNA consists of 200 ng of a 8×CRE-Luc reporter plasmid, 50 ng of pCMV comprising endogenous receptor or non-endogenous receptor or pCMV alone, and 10 ng of a GPRS expression plasmid (GPRS in pcDNA3 (Invitrogen)). The 8×CRE-Luc reporter plasmid is prepared as follows: vector SRIF-β-gal is obtained by cloning the rat somatostatin promoter (−71/+51) at BglV-HindIII site in the pβgal-Basic Vector (Clontech). Eight (8) copies of cAMP response element are obtained by PCR from an adenovirus template AdpCF126CCRE8 (see, Suzuki et al., Hum Gene Ther 7:1883-1893 (1996); the disclosure of which is hereby incorporated by reference in its entirety) and cloned into the SRIF-β-gal vector at the Kpn-BglV site, resulting in the 8×CRE-β-gal reporter vector. The 8×CRE-Luc reporter plasmid is generated by replacing the beta-galactosidase gene in the 8×CRE-β-gal reporter vector with the luciferase gene obtained from the pGL3-basic vector (Promega) at the HindIII-BamHI site. Following 30 minutes incubation at room temperature, the DNA/lipid mixture is diluted with 400 μl of DMEM and 100 μl of the diluted mixture is added to each well. 100 μl of DMEM with 10% FCS are added to each well after a four hour incubation in a cell culture incubator. The following day the transfected cells are changed with 200 μl/well of DMEM with 10% FCS. Eight (8) hours later, the wells are changed to 100 μl/well of DMEM without phenol red, after one wash with PBS. Luciferase activity is measured the next day using the LucLite™ reporter gene assay kit (Packard) following manufacturer instructions and read on a 1450 MicroBeta™ scintillation and luminescence counter (Wallac).

b. AP1 Reporter Assay (Gq-Associated Receptors)

A method to detect Gq stimulation depends on the known property of Gq-dependent phospholipase C to cause the activation of genes containing AP1 elements in their promoter. A Pathdetect™ AP-1 cis-Reporting System (Stratagene, Catalogue No. 219073) can be utilized following the protocol set forth above with respect to the CREB reporter assay, except that the components of the calcium phosphate precipitate are 410 ng pAP1-Luc, 80 ng pCMV-receptor expression plasmid, and 20 ng CMV-SEAP.

c. SRF-LUC Reporter Assay (Gq-Associated Receptors)

One method to detect Gq stimulation depends on the known property of Gq-dependent phospholipase C to cause the activation of genes containing serum response factors in their promoter. A Pathdetect™ SRF-Luc-Reporting System (Stratagene) can be utilized to assay for Gq coupled activity in, for example, COS7 cells. Cells are transfected with the plasmid components of the system and the indicated expression plasmid encoding endogenous or non-endogenous GPCR using a Mammalian Transfection™ Kit (Stratagene, Catalogue #200285) according to the manufacturer's instructions. Briefly, 410 ng SRF-Luc, 80 ng pCMV-receptor expression plasmid and 20 ng CMV-SEAP (secreted alkaline phosphatase expression plasmid; alkaline phosphatase activity is measured in the media of transfected cells to control for variations in transfection efficiency between samples) are combined in a calcium phosphate precipitate as per the manufacturer's instructions. Half of the precipitate is equally distributed over 3 wells in a 96-well plate and kept on the cells in a serum free media for 24 hours. The last 5 hours the cells are incubated with, for example, 1 μM, candidate compound. Cells are then lysed and assayed for luciferase activity using a Luclite™ Kit (Packard, Cat. No. 6016911) and “Trilux 1450 Microbeta” liquid scintillation and luminescence counter (Wallac) as per the manufacturer's instructions. The data can be analyzed using GraphPad Prism™ 2.0a (GraphPad Software Inc.).

d. Intracellular IP3 Accumulation Assay (Gq-Associated Receptors)

On day 1, cells comprising the receptor of interest (endogenous or non-endogenous) can be plated onto 24 well plates, usually 1×10⁵ cells/well (although his number can be optimized). On day 2 cells can be transfected by first mixing 0.25 μg DNA in 50 μl serum free DMEM/well and 2 μl lipofectamine in 50 μl serum free DMEM/well. The solutions are gently mixed and incubated for 15-30 minutes at room temperature. Cells are washed with 0.5 ml PBS and 400 μl of serum free media is mixed with the transfection media and added to the cells. The cells are then incubated for 3-4 hours at 37° C./5% CO₂ and then the transfection media is removed and replaced with 1 ml/well of regular growth media. On day 3 the cells are labeled with ³H-myo-inositol. Briefly, the media is removed and the cells are washed with 0.5 ml PBS. Then 0.5 ml inositol-free/serum free media (GIBCO BRL) is added/well with 0.25 μCi of ³H-myo-inositol/well and the cells are incubated for 16-18 hours overnight at 37° C./5% CO₂. On Day 4 the cells are washed with 0.5 ml PBS and 0.45 ml of assay medium is added containing inositol-free/serum free media, 10 μM pargyline, 10 mM lithium chloride or 0.4 ml of assay medium and 50 μl of 10× ketanserin (ket) to final concentration of 10 μM, if using a control construct containing a serotonin receptor. The cells are then incubated for 30 minutes at 37° C. The cells are then washed with 0.5 ml PBS and 200 μl of fresh/ice cold stop solution (1M KOH; 18 mM Na-borate; 3.8 mM EDTA) is added/well. The solution is kept on ice for 5-10 minutes or until cells were lysed and then neutralized by 200 μl of fresh/ice cold neutralization sol. (7.5% HCL). The lysate is then transferred into 1.5 ml eppendorf tubes and 1 ml of chloroform/methanol (1:2) is added/tube. The solution is vortexed for 15 seconds and the upper phase is applied to a Biorad AG1-X8™ anion exchange resin (100-200 mesh). Firstly, the resin is washed with water at 1:1.25 W/V and 0.9 ml of upper phase is loaded onto the column. The column is washed with 10 mls of 5 mM myo-inositol and 10 ml of 5 mM Na-borate/60 mM Na-formate. The inositol tris phosphates are eluted into scintillation vials containing 10 ml of scintillation cocktail with 2 ml of 0.1 M formic acid/1 M ammonium formate. The columns are regenerated by washing with 10 ml of 0.1 M formic acid/3M ammonium formate and rinsed twice with dd H₂O and stored at 4° C. in water.

### Example 8

### Fusion Protein Preparation

a. GPCR:Gs Fusion Constuct

The design of the GPCR-G protein fusion construct can be accomplished as follows: both the 5′ and 3′ ends of the rat G protein Gsα (long form; Itoh, H. et al., Proc. Natl. Acad. Sci. 83:3776 (1986)) are engineered to include a HindIII sequence thereon. Following confirmation of the correct sequence (including the flanking HindIII sequences), the entire sequence is shuttled into pcDNA3.1 (−) (Invitrogen, cat. no. V795-20) by subcloning using the HindIII restriction site of that vector. The correct orientation for the Gsα sequence is determined after subcloning into pcDNA3.1 (−). The modified pcDNA3.1 (−) containing the rat Gsα gene at HindIII sequence is then verified; this vector is now available as a “universal” Gsα protein vector. The pcDNA3.1 (−) vector contains a variety of well-known restriction sites upstream of the HindIII site, thus beneficially providing the ability to insert, upstream of the Gs protein, the coding sequence of a receptor of interest. This same approach can be utilized to create other “universal” G protein vectors, and, of course, other commercially available or proprietary vectors known to the artisan can be utilized—the important criteria is that the sequence for the GPCR be upstream and in-frame with that of the G protein.

b. Gq(6 Amino Acid Deletion)/Gi Fusion Construct

The design of a Gq(del)/Gi fusion construct can be accomplished as follows: the N-terminal six (6) amino acids (amino acids 2 through 7, having the sequence of TLESIM (SEQ ID NO:3)) of Gαq-subunit is deleted and the C-terminal five (5) amino acids having the sequence EYNLV (SEQ ID NO:4) is replaced with the corresponding amino acids of the Gαi Protein, having the sequence DCGLF (SEQ ID NO:5). This fusion construct can be obtained by PCR using the following primers:

(SEQ ID NO:6)

5′-gatcAAGCTTCCATGGCGTGCTGCCTGAGCGAGGAG-3′
and

(SEQ ID NO:7)

5′-gatcGGATCCTTAGAACAGGCCGCAGTCCTTCAGGTTCAGCTGCAGG
ATGGTG-3′

and Plasmid 63313 which contains the mouse Gαq-wild type version with a hemagglutinin tag as template. Nucleotides in lower caps are included as spacers.

TaqPlus Precision DNA polymerase (Stratagene) can be utilized for the amplification by the following cycles, with steps 2 through 4 repeated 35 times: 95° C. for 2 min; 95° C. for 20 sec; 56° C. for 20 sec; 72° C. for 2 min; and 72° C. for 7 min. The PCR product can be cloned into a pCRII-TOPO vector (Invitrogen) and sequenced using the ABI Big Dye Terminator kit (P.E. Biosystems). Inserts from a TOPO clone containing the sequence of the fusion construct can be shuttled into the expression vector pcDNA3.1 (+) at the HindIII/BamHI site by a 2 step cloning process. Also see, PCT Application Number PCT/US02/05625 published as WO02068600 on 6 Sep. 2002, the disclosure of which is hereby incorporated by reference in its entirety.

### Example 9

### [³⁵S]GTPγS Assay

A. Membrane Preparation

In some embodiments membranes comprising the Target GPCR of interest for use in the identification of candidate compounds as, e.g., agonists, inverse agonists or antagonists, are prepared as follows:

a. Materials

“Membrane Scrape Buffer” is comprised of 20 mM HEPES and 10 nM EDTA, pH 7.4; “Membrane Wash Buffer” is comprised of 20 mM HEPES and 0.1 mM EDTA, pH 7.4; “Binding Buffer” is comprised of 20 mM HEPES, 100 mM NaCl, and 10 mM MgCl₂, pH 7.4.

b. Procedure

All materials are kept on ice throughout the procedure. Firstly, the media is aspirated from a confluent monolayer of cells, followed by rinsing with 10 ml cold PBS, followed by aspiration. Thereafter, 5 ml of Membrane Scrape Buffer is added to scrape cells; this is followed by transfer of cellular extract into 50 ml centrifuge tubes (centrifuged at 20,000 rpm for 17 minutes at 4° C.). Thereafter, the supernatant is aspirated and the pellet is resuspended in 30 ml Membrane Wash Buffer followed by centrifuge at 20,000 rpm for 17 minutes at 4° C. The supernatant is then aspirated and the pellet resuspended in Binding Buffer. This is then homogenized using a Brinkman Polytron™ homogenizer (15-20 second bursts until the all material is in suspension). This is referred to herein as “Membrane Protein”.

Bradford Protein Assay

Following the homogenization, protein concentration of the membranes is determined using the Bradford Protein Assay (protein can be diluted to about 1.5 mg/ml, aliquoted and frozen (−80° C.) for later use; when frozen, protocol for use will be as follows: on the day of the assay, frozen Membrane Protein is thawed at room temperature, followed by vortex and then homogenized with a Polytron at about 12×1,000 rpm for about 5-10 seconds; it is noted that for multiple preparations, the homogenizer should be thoroughly cleaned between homogenization of different preparations).

a. Materials

Binding Buffer (as per above); Bradford Dye Reagent; Bradford Protein Standard is utilized, following manufacturer instructions (Biorad, cat. no. 500-0006).

b. Procedure

Duplicate tubes are prepared, one including the membrane, and one as a control “blank”. Each tube contains 800 μl Binding Buffer. Thereafter, 10 μl of Bradford Protein Standard (1 mg/ml) is added to each tube, and 10 μl of membrane Protein is then added to just one tube (not the blank). Thereafter, 200 μl of Bradford Dye Reagent is added to each tube, followed by vortexing of each tube. After five (5) minutes, the tubes are re-vortexed and the material therein is transferred to cuvettes. The cuvettes are read using a CECIL 3041 spectrophotometer, at wavelength 595.

Identification Assay

a. Materials

GDP Buffer consists of 37.5 ml Binding Buffer and 2 mg GDP (Sigma, cat. no. G-7127), followed by a series of dilutions in Binding Buffer to obtain 0.2 μM GDP (final concentration of GDP in each well is 0.1 μM GDP); each well comprising a candidate compound has a final volume of 200 μl consisting of 100 μl GDP Buffer (final concentration, 0.1 μM GDP), 50 μl Membrane Protein in Binding Buffer, and 50 μl [³⁵S]GTPγS (0.6 nM) in Binding Buffer (2.5 μl [³⁵S]GTPγS per 10 ml Binding Buffer).

b. Procedure

Candidate compounds can be screened using a 96-well plate format (these can be frozen at −80° C.). Membrane Protein (or membranes with expression vector excluding the Target GPCR, as control), are homogenized briefly until in suspension. Protein concentration can be determined using the Bradford Protein Assay set forth above. Membrane Protein (and control) is diluted to 0.25 mg/ml in Binding Buffer (final assay concentration, 12.5 μg/well). Thereafter, 100 μl GDP Buffer is added to each well of a Wallac Scintistrip™ (Wallac). A 5 μl pin-tool is used to transfer 5 μl of a candidate compound into such well (i.e., 5 μl in total assay volume of 200 μl is a 1:40 ratio such that the final screening concentration of the candidate compound is 10 μM). Again, to avoid contamination, after each transfer step the pin tool should be rinsed in three reservoirs comprising water (1×), ethanol (1×) and water (2×)—excess liquid should be shaken from the tool after each rinse and dried with paper and kimwipes. Thereafter, 50 μl of Membrane Protein is added to each well (a control well comprising membranes without the Target GPCR is also utilized), and pre-incubated for 5-10 minutes at room temperature. Thereafter, 50 μl of [³⁵S]GTPγS (0.6 nM) in Binding Buffer is added to each well, followed by incubation on a shaker for 60 minutes at room temperature (plates are covered with foil). The assay is then stopped by spinning of the plates at 4000 RPM for 15 minutes at 22° C. The plates are aspirated with an 8 channel manifold and sealed with plate covers. The plates are read on a Wallac 1450 using setting “Prot. #37” (as per manufacturer's instructions).

### Example 10

### Cyclic AMP Assay

Another assay approach for identifying candidate compounds as, e.g., agonists, inverse agonist, or antagonists, can accomplished by utilizing a cyclase-based assay. In addition to direct identification, this assay approach can be utilized as an independent approach to provide confirmation of the results from the [³⁵S]GTPγS approach as set forth in the above example.

A modified Flash Plate™ Adenylyl Cyclase kit (New England Nuclear; Cat. No. SMP004A) can be utilized for direct identification of candidate compounds as inverse agonists and agonists to a receptor of interest in accordance with the following protocol.

Transfected cells are harvested approximately three days after transfection. Membranes are prepared by homogenization of suspended cells in buffer containing 20 mM HEPES, pH 7.4 and 10 mM MgCl₂. Homogenization is performed on ice using a Brinkman Polytron™ for approximately 10 seconds. The resulting homogenate is centrifuged at 49,000×g for 15 minutes at 4° C. The resulting pellet is then resuspended in buffer containing 20 mM HEPES, pH 7.4 and 0.1 mM EDTA, homogenized for 10 seconds, followed by centrifugation at 49,000×g for 15 minutes at 4° C. The resulting pellet is then stored at −80° C. until utilized. On the day of direct identification screening, the membrane pellet is slowly thawed at room temperature, resuspended in buffer containing 20 mM HEPES, pH 7.4 and 10 mM MgCl₂, to yield a final protein concentration of 0.60 mg/ml (the resuspended membranes are placed on ice until use).

cAMP standards and Detection Buffer (comprising 2 μCi of tracer [¹²⁵I]cAMP (100 μl) to 11 ml Detection Buffer] are prepared and maintained in accordance with the manufacturer's instructions. Assay Buffer is prepared fresh for screening and contains 20 mM HEPES, pH 7.4, 10 mM MgCl₂, 20 mM phosphocreatine (Sigma), 0.1 units/ml creatine phosphokinase (Sigma), 50 μM GTP (Sigma), and 0.2 mM ATP (Sigma); Assay Buffer is then stored on ice until utilized.

Candidate compounds are added to, for example, 96-well plate wells (3 μl/well; 12 μM final assay concentration), together with 40 μl Membrane Protein (30 μg/well) and 50 μl of Assay Buffer. This admixture is then incubated for 30 minutes at room temperature, with gentle shaking.

Following the incubation, 100 μl of Detection Buffer is added to each well, followed by incubation for 2-24 hours. Plates are then counted in a Wallac MicroBeta™ plate reader using “Prot. #31” (as per manufacturer's instructions).

### Example 11

### Fluorometric Imaging Plate Reader (FLIPR) Assay for the Measurement of Intracellular Calcium Concentration

Target Receptor (experimental) and pCMV (negative control) stably transfected cells from respective clonal lines are seeded into poly-D-lysine pretreated 96-well plates (Becton-Dickinson, #356640) at 5.5×10⁴ cells/well with complete culture medium (DMEM with 10% FBS, 2 mM L-glutamine, 1 mM sodium pyruvate) for assay the next day. Because the niacin receptor is Gi coupled, the cells comprising the niacin receptor can further comprise Gα15, Gα16, or the chimeric Gq/Gi alpha subunit. To prepare Fluo-4-AM (Molecular Probe, #F 14202) incubation buffer stock, 1 mg Fluo-4-AM is dissolved in 467 μl DMSO and 467 μl Pluoronic acid (Molecular Probe, #P3000) to give a 1 mM stock solution that can be stored at −20° C. for a month. Fluo-4-AM is a fluorescent calcium indicator dye.

Candidate compounds are prepared in wash buffer (1×HBSS/2.5 mM Probenicid/20 mM HEPES at pH 7.4).

At the time of assay, culture medium is removed from the wells and the cells are loaded with 100 μl of 4 μM Fluo-4-AM/2.5 mM Probenicid (Sigma, #P8761)/20 mM HEPES/complete medium at pH 7.4. Incubation at 37° C./5% CO₂ is allowed to proceed for 60 minutes.

After the 1 hour incubation, the Fluo-4-AM incubation buffer is removed and the cells are washed 2× with 100 μl wash buffer. In each well is left 100 μl wash buffer. The plate is returned to the incubator at 37° C./5% CO₂ for 60 minutes.

FLIPR (Fluorometric Imaging Plate Reader; Molecular Device) is programmed to add 50 μl candidate compound on the 30th second and to record transient changes in intracellular calcium concentration ([Ca2+]) evoked by the candidate compound for another 150 seconds. Total fluorescence change counts are used to determine agonist activity using the FLIPR software. The instrument software normalizes the fluorescent reading to give equivalent initial readings at zero.

Although the foregoing provides a FLIPR assay for agonist activity using stably transfected cells, a person of ordinary skill in the art would readily be able to modify the assay in order to characterize antagonist activity. Said person of ordinary skill in the art would also readily appreciate that, alternatively, transiently transfected cells could be used.

### Example 12

### Receptor Binding Assay

In addition to the methods described herein, another means for evaluating a candidate compound is by determining binding affinities to the niacin receptor. This type of assay generally requires a radiolabelled ligand to the niacin receptor.

A radiolabelled compound such as radiolabelled niacin can be used in a screening assay to identify/evaluate compounds. In general terms, a newly synthesized or identified compound (i.e., candidate compound) can be evaluated for its ability to reduce binding of the radiolabelled niacin to the niacin receptor. Accordingly, the ability to compete with the radiolabelled niacin for the binding to the niacin receptor directly correlates to the binding affinity of the candidate compound to the niacin receptor.

### Assay Protocol for Determining Receptor Binding for the Niacin Receptor:

A. Niacin Receptor Preparation

For example, HEK293 cells (human kidney, ATCC) can be transiently or stably transfected with the niacin receptor as described herein. For example, 293 cells can be transiently transfected with 10 μg human niacin receptor and 60 μl Lipofectamine (per 15-cm dish), and grown in the dish for 24 hours (75% confluency) with a media change. Cells are removed with 10 ml/dish of Hepes-EDTA buffer (20 mM Hepes+10 mM EDTA, pH 7.4). The cells are then centrifuged in a Beckman Coulter centrifuge for 20 minutes, 17,000 rpm (JA-25.50 rotor). Subsequently, the pellet is resuspended in 20 mM Hepes+1 mM EDTA, pH 7.4 and homogenized with a 50-ml Dounce homogenizer and again centrifuged. After removing the supernatant, the pellets are stored at −80° C., until used in binding assay. When used in the assay, membranes are thawed on ice for 20 minutes and then 10 mL of incubation buffer (20 mM Hepes, 1 mM MgCl₂, 100 mM NaCl, pH 7.4) is added. The membranes are then vortexed to resuspend the crude membrane pellet and homogenized with a Brinkmann PT-3100 Polytron homogenizer for 15 seconds at setting 6. The concentration of membrane protein is determined using the BRL Bradford protein assay.

B. Binding Assay

For total binding, a total volume of 50 μl of appropriately diluted membranes (diluted in assay buffer containing 50 mM Tris HCl (pH 7.4), 10 mM MgCl₂, and 1 mM EDTA; 5-50 μg protein) is added to 96-well polyproylene microtiter plates followed by addition of 100 μl of assay buffer and 501 of radiolabelled niacin. For nonspecific binding, 50 μl of assay buffer is added instead of 100 μl and an additional 50 μl of 10 μM cold niacin receptor is added before 50 μl of radiolabelled niacin is added. Plates are then incubated at room temperature for 60-120 minutes. The binding reaction is terminated by filtering assay plates through a Microplate Devices GF/C Unifilter filtration plate with a Brandell 96-well plate harvestor followed by washing with cold 50 mM Tris HCl, pH 7.4 containing 0.9% NaCl. Then, the bottom of the filtration plates are sealed, 50 μl of Optiphase Supermix is added to each well, the top of the plates are sealed, and plates are counted in a Trilux MicroBeta scintillation counter. For compound competition studies, instead of adding 100 μl of assay buffer, 100 μl of appropriately diluted candidate compound is added to appropriate wells followed by addition of 50 μl of radiolabelled niacin.

C. Calculations

The candidate compounds are initially assayed at 1 and 0.1 μM and then at a range of concentrations chosen such that the middle dose would cause about 50% inhibition of a radiolabelled niacin binding (i.e., IC₅₀). Specific binding in the absence of candidate compound (B_{O}) is the difference of total binding (B_{T}) minus non-specific binding (NSB) and similarly specific binding (in the presence of candidate compound) (B) is the difference of displacement binding (B_{D}) minus non-specific binding (NSB). IC₅₀ is determined from an inhibition response curve, logit-log plot of % B/B_{O} vs concentration of candidate compound.

Kᵢ is calculated by the Cheng and Prustoff transformation:

Kᵢ=IC₅₀/(1+[L]/K_{D}).

where [L] is the concentration of a radiolabelled niacin used in the assay and K_{D} is the dissociation constant of a radiolabelled niacin determined independently under the same binding conditions.

### Example 13

### Preparation of Compounds of the Invention

### Example 13.1

### Preparation of 3-(1H-Tetrazol-5-yl)-1,4,5,6-tetrahydro-cyclopentapyrazole (Compound 1)

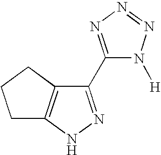

### Method A: Preparation of Compound 1.

1,4,5,6-Tetrahydro-cyclopentapyrazole-3-carbonitrile (0.022 g, 0.165 mmol) and sodium azide (0.086 g, 1.30 mmol) were taken up in DMF (3 cm³) at heated under microwave irradiation to 175° C. for 20 minutes. The solution was cooled to room temperature, filtered and the filtered solid washed with ethyl acetate. The combined solutions was added to saturated aqueous sodium bicarbonate (20 cm³) and washed with ethyl acetate. The aqueous layer was acidified to pH 1 with the addition of 1M aqueous hydrochloric acid and extracted into ethyl acetate. The ethyl acetate washes were combined and solvent removed under reduced pressure, the resulting solid purified by preparative HPLC to give 3-(2H-tetrazol-5-yl)-1,4,5,6-tetrahydro-cyclopentapyrazole as a white solid (0.012 g, 0.068 mmol, 41%). ¹H NMR δ (CD₃OD): 2.88 (t-like, 2H, J=7.0), 2.82 (t-like, 2H, J=7.3), 2.64 (quintet-like, 2H, J=7.1); m/z (ES⁺): 177 [M+H]⁺.

The intermediate 1,4,5,6-Tetrahydro-cyclopentapyrazole-3-carbonitrile was prepared using the following procedure.

### Step A: 1,4,5,6-Tetrahydro-cyclopentapyrazole-3-carboxylic acid ethyl ester

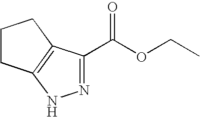

Cyclopentanone (10.0 g, 118.9 mmol) was taken up in absolute ethanol (30 cm³) and sodium ethoxide (53 cm³, 21% in ethanol, 143 mmol) was added. The resulting solution was stirred under argon for 10 minutes, then diethyl oxalate (19.1 g, 131 mmol) added. Further ethanol (10 cm³) was added and the solution heated at 75° C. for 3 hours and cooled to room temperature. Hydrazine hydrochloride (8.15 g, 119 mmol), taken up in water (20 cm³) was added and the solution heated to 75° C. overnight. Solvent was removed under reduced pressure and the resulting taken up in ethyl acetate (200 cm³) and washed with water (200 cm³), dried (Na₂SO₄), filtered and solvent removed under reduced pressure to give 1,4,5,6-tetrahydro-cyclopentapyrazole-3-carboxylic acid ethyl ester as an off white solid (16.16 g, 90.0 mmol, 76%). δ_{H} (CD₃OD): 4.34 (q, 2H, J=7.1, OCH₂CH₃), 2.78 (t like, 2H, J=7.0), 2.72 (br s, 2H), 2.49 (br s, 2H), 1.36 (t, 3H, J=7.1, OCH₂CH₃). m/z (ES⁺): 181 [M+H]⁺.

### Step B: 1,4,5,6-Tetrahydro-cyclopentapyrazole-3-carboxylic acid amide

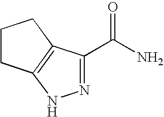

1,4,5,6-Tetrahydro-cyclopentapyrazole-3-carboxylic acid ethyl ester (0.808 g, 4.48 mmol) was taken up in methanolic ammonia (ca 7 M, 12 cm³) and stirred overnight at 95° C. The resulting solution was chilled and the precipitated 1,4,5,6-tetrahydro-cyclopentapyrazole-3-carboxylic acid amide collected by vacuum filtration as a white crystalline solid (0.438 g, 2.90 mmol, 65%). δ_{H} (CD₃OD): 2.79 (t like, 2H, J=6.9), 2.73 (t like, 2H, J=7.3), 2.55 (br s, 2H); m/z (ES⁺): 152 [M+H]⁺.

### Step C: 1,4,5,6-Tetrahydro-cyclopentapyrazole-3-carbonitrile

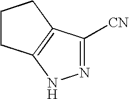

1,4,5,6-Tetrahydro-cyclopentapyrazole-3-carboxylic acid amide (0.210 g, 1.39 mmol) was added to anhydrous acetonitrile (12 cm³), heated to 80° C. and sodium chloride (2.0 g, 34 mmol) added. After 15 minutes phosphorus oxychloride (0.128 g, 0.83 mmol) was added and the solution heated to 80° C. overnight, cooled, filtered, and the collected solid washed with acetonitrile. Solvent was removed from the combined solutions under reduced pressure and the resulting solid purified by preparative HPLC to give 1,4,5,6-tetrahydro-cyclopentapyrazole-3-carbonitrile as a deep purple coloured solid (0.031 g, 0.23 mmol, 17%). δ_{H} (CD₃OD): 2.79 (t like, 2H, J=7.3), 2.73 (t like, 2H, J=7.1), 2.65-2.55 (m, 2H); m/z (ES): 134 [M+H]⁺.

### Method B: Preparation of Compound 1.

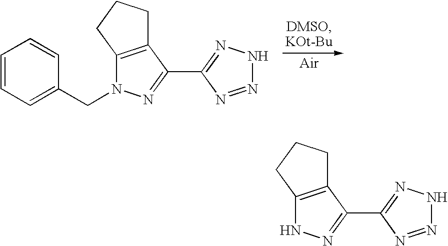

Air was bubbled through a stirring solution of 1-benzyl-3-(2H-tetrazol-5-yl)-1,4,5,6-tetrahydro-cyclopentapyrazole (1.92 g, 7.21 mmol) and KOt-Bu (65 mL of a 1M solution in THF) in DMSO (50 mL) for a period of 2.0 h. The reaction was acidified to pH=2 by the addition of HCl (3M aq). The mixture was filtered and the filtrate was concentrated in vacuo to remove volatiles. The material was purified by reverse-phase HPLC: Phenomenex® Luna C18 column (10μ, 250×50 mm), 5% (v/v) CH₃CN (containing 1% v/v TFA) in H₂O (containing 1% v/v TFA) gradient to 50% H₂O, 60 ml/min, λ=214 nm. The product was further purified by loading material on a Varian BondElut® 60 mL, 10 g SCX cartridge. MeOH (150 mL) was passed through the column to remove unbound impurities. The product was then eluted by passing a solution of 2N NH₃ in MeOH (150 mL) through the column. Concentration of the eluant yielded the ammonium salt of Compound 1 (947 mg, 5.38 mmol, 75% yield) as a white solid. ¹H NMR (ammonium salt, 400 MHz, CD₃OD): δ 2.88 (2H, t, J=6.8 Hz), 2.74 (2H, t, J=6.8 Hz), 2.52 (2H, quin, J=6.8 Hz). HPLC/MS: Discovery® C18 column (5μ, 50×2.1 mm), 5% v/v CH₃CN (containing 1% v/v TFA) in H₂O (containing 1% v/v TFA) gradient to 99% v/v CH₃CN in H₂O, 0.75 mL/min, tᵣ=1.22 min, ESI⁺=177.3 (M+H). Anal Calcd for C₇H₈N₆ (neutral compound): C, 47.72; H, 4.58. Found: C, 47.27; H, 4.16. Anal Calcd for C₇H₁₁N₇ (ammonium salt): C, 43.51; H, 5.74. Found: C, 42.94; H, 5.30.

The intermediate 1-benzyl-3-(2H-tetrazol-5-yl)-1,4,5,6-tetrahydro-cyclopentapyrazole was prepared using the following procedure.

### Step A: Preparation of 1-Benzyl-1,4,5,6-tetrahydro-cyclopentapyrazole-3-carboxylic acid amide and 2-Benzyl-2,4,5,6-tetrahydro-cyclopentapyrazole-3-carboxylic acid amide

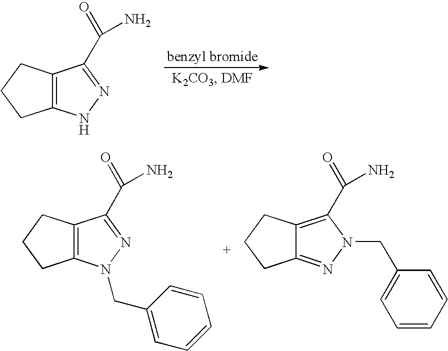

To a stirring solution of 1,4,5,6-tetrahydro-cyclopentapyrazole-3-carboxylic acid amide (2.57 g, 17.0 mmol) in DMF (34 mL) at 25° C. was added K₂CO₃ (5.87 g, 42.5 mmol) followed by benzyl bromide (4.36 g g, 25.5 mmol). The reaction was stirred at ambient temperature for 16 h at which time the mixture was diluted with EtOAc (75 mL) and filtered. The filtrate was washed with H₂O (100 mL) and the aqueous phase was back-extracted with EtOAc (75 mL) and CH₂Cl₂ (75 mL). The combined organic extracts were dried over MgSO₄, filtered, and concentrated in vacuo. Purification by silica gel chromatography (50% EtOAc in hexanes gradient to 95% EtOAc in hexanes) gave 2-benzyl-2,4,5,6-tetrahydro-cyclopentapyrazole-3-carboxylic acid amide (739 mg, 3.07 mmol, 18% yield) isolated as a white solid followed by 1-benzyl-1,4,5,6-tetrahydro-cyclopentapyrazole-3-carboxylic acid amide (3.24 g, 13.4 mmol, 79% yield) isolated as a white solid.

### 1-Benzyl-1,4,5,6-tetrahydro-cyclopentapyrazole-3-carboxylic acid amide

¹H NMR (400 MHz, CDCl₃): δ 7.37-7.30 (3H, m), 7.19 (2H, m), 6.67 (1H, bs), 5.34 (1H, bs), 5.19 (2H, s), 2.82 (2H, m), 2.51 (4H, m). ¹³C APT NMR (100 MHz, CDCl₃): δ up: 164.8, 155.2, 139.0, 136.0, 129.5, 55.3, 31.2, 24.1; down: 129.0, 128.3, 127.8. HPLC/MS: Alltech® Prevail C18 column (5μ, 50×4.6 mm), 5% v/v CH₃CN (containing 1% v/v TFA) in H₂O (containing 1% v/v TFA) gradient to 99% v/v CH₃CN in H₂O, 3.5 mL/min, tᵣ=2.13 min, ESI⁺=242.2 (M+H).

### 2-Benzyl-2,4,5,6-tetrahydro-cyclopentapyrazole-3-carboxylic acid amide

¹H NMR (400 MHz, CDCl₃): δ 7.34-7.21 (5H, m), 5.76 (2H, s), 5.70-5.38 (2H, bs), 2.78 (4H, m), 2.49 (2H, m). ¹³C APT NMR (100 MHz, CDCl₃): δ up: 161.9, 160.1, 138.3, 128.3, 127.1, 55.1, 29.9, 24.8, 24.7; down: 128.6, 128.0, 127.6. HPLC/MS: Alltech® Prevail C18 column (5μ, 50×4.6 mm), 5% v/v CH₃CN (containing 1% V/V TFA) in H₂O (containing 1% v/v TFA) gradient to 99% v/v CH₃CN in H₂O, 3.5 mL/min, tᵣ=1.98 min, ESI⁺=242.1 (M+H).

### Step B: Preparation of 1-Benzyl-3-(2H-tetrazol-5-yl)-1,4,5,6-tetrahydro-cyclopentapyrazole

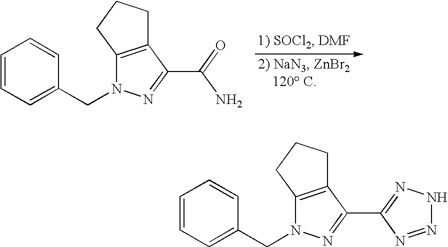

To a solution of 1-benzyl-1,4,5,6-tetrahydro-cyclopentapyrazole-3-carboxylic acid amide (3.02 g, 12.53 mmol) in DMF (25 mL) at rt was added thionyl chloride (1.94 g, 16.3 mmol). The reaction was stirred for 18 h at which time NaHCO₃ (sat. aq., 6 mL) was added to quench excess thionyl chloride. The mixture was diluted with EtOAc (150 mL) and washed sequentially with NaHCO₃ (sat. aq., 100 mL) and brine (100 mL). The aqueous washes were back-extracted with EtOAc (2×100 mL) and the combined organics were dried over MgSO₄, filtered, and concentrated in vacuo to yield a crude yellow oil.

The concentrate was dissolved in DMF (20 mL) and placed in a heavy walled sealed reaction vessel at which time to which ZnBr₂ (4.70 g, 18.0 mmol) and NaN₃ (2.73 g, 42.0 mmol) were added sequentially. The vessel was sealed and heated to 120° C. for 18 h. The mixture was cooled to rt and HCl (3M aq., 2 mL) was added and stirring was continued for 5 min. The mixture was diluted with EtOAc (150 mL) and washed with HCl (1M, aq., 100 mL). The organics were dried over MgSO₄, filtered, and concentrated. Purification by silica gel chromatography (50:50:0.2, hexanes: EtOAc:AcOH gradient to 100:0.2, EtOAc: AcOH) gave 1-benzyl-3-(2H-tetrazol-5-yl)-1,4,5,6-tetrahydro-cyclopentapyrazole (2.06 g, 7.74 mmol, 62% yield) as a white solid. ¹H NMR (400 MHz, CD₃OD): δ 7.36-7.25 (5H, m), 5.30 (2H, s), 2.84 (2H, t, J=6.4 Hz), 2.62-2.56 (4H, m). ¹³C APT NMR (100 MHz, CD₃OD): δ up: 153.8, 151.9, 137.6, 131.5, 128.9, 55.8, 31.9, 24.8, 24.6; down: 129.9, 129.1, 129.0. HPLC/MS: Discovery® C18 column (5μ, 50×2.1 mm), 5% v/v CH₃CN (containing 1% v/v TFA) in H₂O (containing 1% v/v TFA) gradient to 99% v/v CH₃CN in H₂O, 0.75 mL/min, tᵣ=2.18 min, ESI⁺=267.1 (M+H).

### Method C: Preparation of Compound 1.

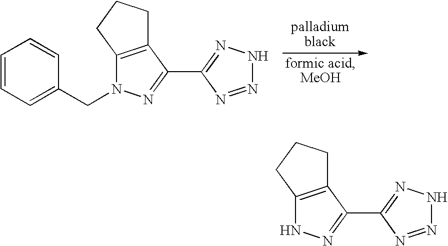

To a solution of 1-benzyl-3-(2H-tetrazol-5-yl)-1,4,5,6-tetrahydro-cyclopentapyrazole (59.4 g, 223 mmol) in 10% formic acid/MeOH (vol/vol, 900 mL) was added palladium black (39.8 g, 374 mmol). The mixture was mechanically stirred under N₂ atmosphere for 24 h. The reaction was filtered and concentrated. The product was further purified and converted to the ammonium salt by the following by loading material (as a solution in MeOH) on to a column containing Bondesil SCX SPE resin (750 g). The column was flushed with MeOH (2.0 L) to remove unbound impurities. The product was eluted using 2N NH₃/MeOH (approx. 1.5 L). Upon concentration the ammonium salt of the tetrazole (39.3 g, 203 mmol, 91% yield) was obtained as a white solid.

The intermediate 1-benzyl-3-(2H-tetrazol-5-yl)-1,4,5,6-tetrahydro-cyclopentapyrazole was prepared using the following procedure.

### Step A: Preparation of 1,4,5,6-Tetrahydro-cyclopentapyrazole-3-carboxylic acid ethyl ester

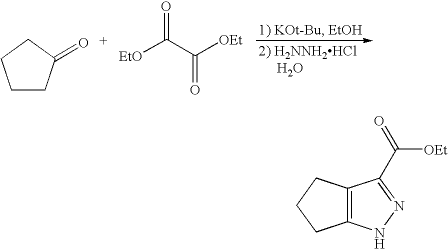

To a solution of cyclopentanone (42.0 g, 0.50 mol) and diethyl oxalate (73.1 g, 0.50 mol) in EtOH (2.5 L) at rt under N₂ was added a solution of KOt-Bu in THF (500 mL of a 1M solution, 0.50 mol) over 0.5 h via an addition funnel. The reaction was stirred for 3.5 h at which time the flask was cooled to 0° C. Hydrazine hydrochloride (37.6 g, 0.55 mol) in H₂O (250 mL) was added via addition funnel over 0.5 h. The reaction was warmed to rt and stirred for 16 h. The volatiles were removed in vacuo and the resulting solid was washed with NaHCO₃ (sat. aq., 500 mL) and H₂O (500 mL). Further concentration in vacuo gave pure 1,4,5,6-tetrahydro-cyclopentapyrazole-3-carboxylic acid ethyl ester (63.6 g, 0.35 mol, 71% yield) as a yellow solid.

### Step B: Preparation of 1,4,5,6-tetrahydro-cyclopentapyrazole-3-carboxylic acid amide

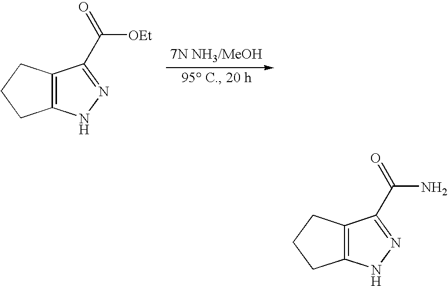

1,4,5,6-Tetrahydro-cyclopentapyrazole-3-carboxylic acid ethyl ester (63.5 g, 0.35 mmol) was dissolved in a solution of 7N NH₃/MeOH (1.0 L). The solution was divided into four equal portions each of which was transferred to 350 mL heavy-walled sealed reaction vessel. The vessels were heated to 95° C. and stirred for 20 h. The reactions were cooled to rt at which time a solid precipitated. The solution was filtered and the solid was washed with NaOH (1N aq., 200 mL) giving pure 1,4,5,6-tetrahydro-cyclopentapyrazole-3-carboxylic acid amide (42.0 g, 0.20 mol, 80% yield) as a white solid.

### Step C: Preparation of 1-Benzyl-1,4,5,6-tetrahydro-cyclopentapyrazole-3-carboxylic acid amide and 2-Benzyl-1,4,5,6-tetrahydro-cyclopentapyrazole-3-carboxylic acid amide

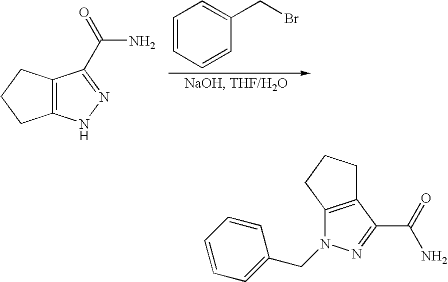

To a solution of 1,4,5,6-tetrahydro-cyclopentapyrazole-3-carboxylic acid amide (41.5 g, 275 mmol) in THF (460 mL) at rt was added a solution of NaOH (5N aq., 110 mL, 0.54 mol). After stirring for 5 min benzyl bromide (49.2 g, 0.29 mol) was added and the reaction was stirred for 16 h. The volatiles were removed in vacuo and the resulting solid was washed with H₂O (3×250 mL). Further concentration gave regioisomers of 1-benzyl-1,4,5,6-tetrahydro-cyclopentapyrazole-3-carboxylic acid amide and 2-benzyl-1,4,5,6-tetrahydro-cyclopentapyrazole-3-carboxylic acid amide (65.3 g, 270 mmol, 98% yield) as a 20:1 mixture and was used without separation).

### Step D: Preparation of 1-Benzyl-3-(2H-tetrazol-5-yl)-1,4,5,6-tetrahydro-cyclopentapyrazole

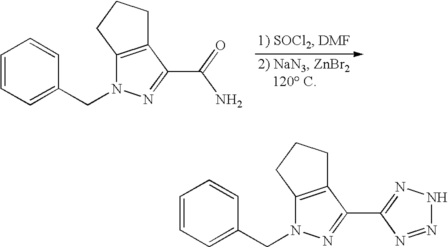

A flask equipped with a drying tube under N₂ atmosphere was charged with anhydrous DMF (250 mL). The flask was cooled to 0° C. and thionyl chloride (36.7 g, 309 mmol) was added via syringe over a period of 5 min. After stirring for an additional 10 min, a solution of 1-benzyl-1,4,5,6-tetrahydro-cyclopentapyrazole-3-carboxylic acid amide (67.7 g, 2811 mmol) in DMF (310 mL) was added over 5 min using an addition funnel. The mixture was slowly warmed to rt and stirred for 16 hr. NaHCO₃ (sat. aq., 100 mL) was added and the mixture was stirred for 10 min. The volatiles were removed in vacuo and the residue was diluted with EtOAc (700 mL) and NaHCO₃ (sat. aq., 700 mL). The layers were separated and the aqueous phase was back-extracted with EtOAc (400 mL). The combined organics were washed with NaHCO₃ (sat. aq., 600 mL) and brine (600 mL), dried over MgSO₄, filtered, and concentrated to give 63.1 g of nitrile as a brown solid.

To a solution of the nitrile (from above) in DMF (560 mL) was added ZnBr₂ (95.6 g, 425 mmol) followed by NaN₃ (55.2 g, 849 mmol). The mixture was heated to 120° C. and stirred for 14 h. The reaction was cooled to rt and the DMF was removed in vacuo. HCl (2N aq., 800 mL) was added and the mixture was stirred for 15 min followed by filtration. The solid was added to a biphasic mixture of EtOAc (500 mL) and HCl (5N aq., 300 mL) and stirred for 0.5 h. The solution was filtered and the layers separated. The remaining solid was again treated with EtOAc and HCl (5N aq.) as described above and this process (stir, filter, separate) was repeated until all solid material was dissolved. The combined organic filtrates were concentrated to give 1-benzyl-3-(2H-tetrazol-5-yl)-1,4,5,6-tetrahydro-cyclopentapyrazole (61.0 g, 229 mmol, 81% yield from the amide) as a light brown solid.

### Example 13.2

### General Procedure for the Preparation of Pyrazoles Carboxylic Acids of the Invention

To a corresponding ketone dissolved in ethanol (5 mL/mmol), is added diethyl oxalate (1.2 eq.) and 1M solution t-BuOK in THF(1.1 eq.). The mixture is heated at 75° C. for 30 minutes, then cooled to 4° C. in a ice bath. An aqueous solution of Hydrazine (2 eq., 2 mL/mmol) is added and the resulting mixture is heated at 75° C. for 1 hour. Ethanol is removed under reduced pressure and the crude is diluted with a saturated aqueous solution of NaHCO₃ and extracted with EtOAc. The organic layer is dried over Na₂SO₄ and concentrated to give the corresponding pyrazole ester derivative. Subsequently, the hydrolysis of the ester is performed under basic condition using 5N aqueous solution NaOH at 95° C. over a period of 2 h. The pH of the solution is adjusted to ˜1 using a 12N HCl and the mixture extracted with AcOEt, the organic layer is dried over Na₂SO₄ and concentrated. The crude material is purified by crystallization or HPLC to afford the pyrazole carboxylic acid derivative.

### Example 13.3

### Preparation of 5-(3-Fluoro-benzyl)-1H-pyrazole-3-carboxylic acid (Compound 2)

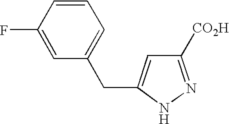

5-(3-Fluoro-benzyl)-1H-pyrazole-3-carboxylic acid was prepared using the general procedure as described in Example 13.2. ¹H NMR (DMSO, 400 MHz) δ (ppm): 7.34 (1H, m), 7.07 (2H, m), 6.50 (1H, s), 3.98 (2H, s). Mass Spectrum: m/z: 221 (M+1)⁺.

### Example 13.4

### Preparation of 5-(3-Chloro-benzyl)-1H-pyrazole-3-carboxylic acid (Compound 3)

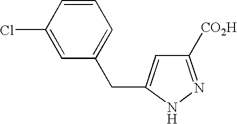

5-(3-Chloro-benzyl)-1H-pyrazole-3-carboxylic acid was prepared using the general procedure as described in Example 13.2.

### Example 13.5

### Preparation of 5-(3-Bromo-benzyl)-1H-pyrazole-3-carboxylic acid (Compound 4)

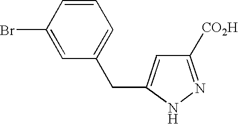

5-(3-Bromo-benzyl)-1H-pyrazole-3-carboxylic acid was prepared using the general procedure as described in Example 13.2. ¹H NMR (DMSO, 400 MHz) δ (ppm): 7.46 (1H, s), 7.42 (1H, m), 7.27 (2H, m), 6.51 (1H, s), 3.97 (2H, s). Mass Spectrum: m/z: 281 (M+1)+, 283 (M+1)⁺.

### Example 13.6

### Preparation of 6-Methyl-3-(1H-tetrazol-5-yl)-4,6-dihydro-1H-furo[3,4-c]pyrazole (Compound 5)

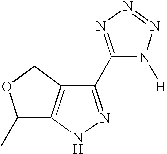

6-Methyl-3-(1H-tetrazol-5-yl)-4,6-dihydro-1H-furo[3,4-c]pyrazole was prepared in a similar manner as described in Example 13.1, a separation by column chromatography of the regioisomers was performed after the formation of the pyrazole. Compound 5 was characterized by NMR and MS; ¹H NMR (400 MHz, DMSO): δ 5.20 (m, 1H), 4.94 (dd, J=34.7, 10.3 Hz, 2H), 1.39 (d, J=4.4 Hz, 3H). HPLC/MS: Alltech® Prevail C18 column (5%, 50×4.6 mm), 5% v/v CH₃CN (containing 1% v/v TFA) in H₂O (containing 1% v/v TFA) gradient to 99% v/v CH₃CN in H₂O, 3.5 mL/min, tᵣ=1.03 min, ESI⁺=192 (M+H).

### Example 13.7

### Preparation of 3-(1H-Tetrazol-5-yl)-4,6-dihydro-1H-thieno[3,4-c]pyrazole (Compound 6)

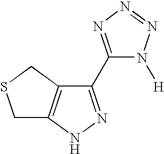

3-(1H-Tetrazol-5-yl)-4,6-dihydro-1H-thieno[3,4-c]pyrazole was prepared in a similar manner as described in Example 13.1, and was characterized by NMR and MS; ¹H NMR (400 MHz, MeOD): δ 4.00 (2H, m), 3.95 (2H, m). HPLC/MS: Waters® YMC ODS-A C18 column (5A, 50×4.6 mm), 5% v/v CH₃CN (containing 1% v/v TFA) in H₂O (containing 1% v/v TFA) gradient to 99% v/v CH₃CN in H₂O, 3.5 mL/min, tᵣ=1.27 min, ESI⁺=194 (M+H).

### Example 13.8

### Preparation of 3-(1H-Tetrazol-5-yl)-1,4-dihydro-cyclopentapyrazole (Compound 7) and 3-(1H-Tetrazol-5-yl)-1,6-dihydro-cyclopentapyrazole (Compound 8)

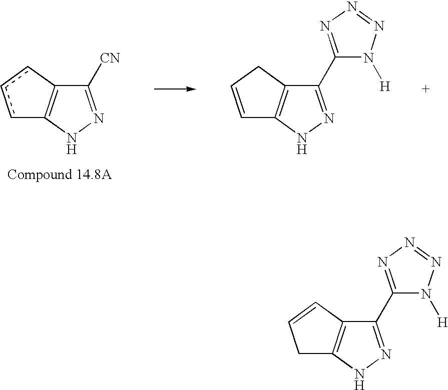

### Compound 13.8A

A solution of Compound 13.8A, as an isomeric mixture, (50 mg, 0.38 mmol), sodium azide (86.5 mg, 1.33 mmol) and zinc bromide (300 mg, 1.33 mmol) in DMF (2 mL) was irradiated under microwave at 200° C. for 6 hours. After cooling to room temperature, the reaction mixture was treated with a 2 N HCl solution, extracted with EtOAc, washed with H₂O and concentrated in vacuo. HPLC separation (C18 column, 5 to 99% CH₃CN in H₂O) afforded 40.3 mg (61%) of the desired products as a 2:1 mixture of olefinic isomers. LC-MS m/z 175 (M+1); ¹H NMR (400 MHz, DMSO-d₆) δ 6.94 (m, 0.5H), 6.87 (m, 1H), 6.76 (m, 1H), 6.40 (m, 0.5H), 3.35 (m, 3H).

The isomers were separated by reverse-phase HPLC: Phenomenex® Luna C18 column (10μ, 250×21.2 mm), 5% (v/v) CH₃CN (containing 1% v/v TFA) in H₂O (containing 1% v/v TFA) gradient to 70% H₂O, 20 ml/min, λ=280 nm.

Alternatively the isomers were separated by normal-phase HPLC: Dynamax Micorsorb Si (prep) column (8μ, 250×10 mm), 80% (v/v) EtOAc (containing 2% v/v AcOH) in hexanes (containing 2% v/v AcOH) gradient to 99% EtOAc, 7.5 ml/min, μ=280 nm.

### The Order of Isomer Elution is the Same for Both Normal- and Reverse-Phase Columns.

Isomer 1 (High Rf Isomer):

¹H NMR (400 MHz, MeOD): δ 6.79 (2H, m), 3.42 (2H, m). HPLC/MS: Discovery® C18 column (5μ, 50×2.1 mm), 5% v/v CH₃CN (containing 1% v/v TFA) in H₂O (containing 1% v/v TFA) gradient to 99% v/v CH₃CN in H₂O, 0.75 mL/min, tᵣ=1.10 min, ESI⁺=174.9 (M+H).

Isomer 2 (Low Rf Isomer):

¹H NMR (400 MHz, MeOD): δ 6.98 (1H, m), 6.44 (1H, m), 3.33 (2H, m). HPLC/MS: Discovery® C18 column (5μ, 50×2.1 mm), 5% v/v CH₃CN (containing 1% v/v TFA) in H₂O (containing 1% v/v TFA) gradient to 99% v/v CH₃CN in H₂O, 0.75 mL/min, tᵣ=1.11 min, ESI⁺=175.1 (M+H).

The intermediate Compound 13.8A, as an isomeric mixture, was prepared using the following steps:

### Step A: Preparation of 2,4-Dihydro-cyclopentapyrazole-3-carboxylic acid ethyl ester and 2,6-Dihydro-cyclopentapyrazole-3-carboxylic acid ethyl ester (mixture)

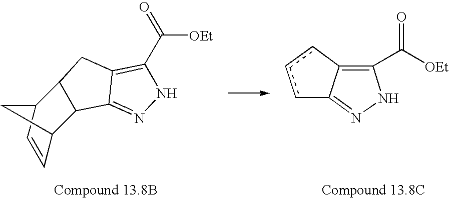

Compound 13.8B was prepared from the corresponding ketone using a similar method as described herein for the preparation of pyrazole esters (see Example 13.1 and 13.2). A solution of Compound 13.8B (2.0 g, 8.19 mmol) in phenyl ether (25 mL) was heated at reflux (250˜260° C.) under nitrogen for 2 hours.

After cooling down the solution to room temperature, it was loaded on a SiO₂ column, flushed with DCM to push out the phenyl ether, and eluted with EtOAc/Hex (1/3) to afford 1.05 g (72%) of Compound 13.8C as a mixture of olefinic isomers. LC-MS m/z 179 (M+1).

### Step B: Preparation of 2,4-Dihydro-cyclopentapyrazole-3-carboxylic acid amide and 2,6-Dihydro-cyclopentapyrazole-3-carboxylic acid amide (mixture)

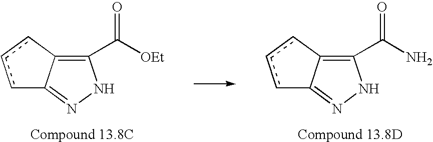

Compound 13.8C, as an isomeric mixture, (1.0 g, 5.61 mmol) was dissolved in smallest amount of dioxane (<5 mL) and mixed with 28% ammonium hydroxide solution (100 mL) in a tightly sealed container. The solution was stirred at room temperature for 24 hours and concentrated in vacuo to afford Compound 13.8D, as an isomeric mixture, as a solid in quantitative yield. LC-MS m/z 150 (M+1).

### Step C: Preparation of 2,4-Dihydro-cyclopentapyrazole-3-carbonitrile and 2,6-Dihydro-cyclopentapyrazole-3-carbonitrile (mixture)

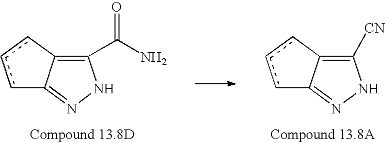

To a suspension of Compound 13.8D, as an isomer mixture, (0.80 g, 5.36 mmol) and potassium carbonate (0.445 g, 3.22 mmol) in acetonitrile (30 mL) was added POCl₃ (0.785 mL, 8.58 mmol) at room temperature. The reaction mixture was heated at reflux for 2 hours. After concentration in vacuo, the residue was diluted with EtOAc (150 mL), washed with H₂O and brine, dried (Na₂SO₄), and concentrated to afford 141 mg (20%) of Compound 13.8A as an isomer mixture. LC-MS m/z 132 (M+1).

### Example 13.9

### Preparation of 3-(1H-Tetrazol-5-yl)-4,6-dihydro-1H-furo[3,4-c]pyrazole (Compound 9)

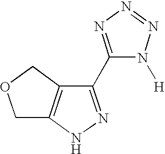

Compound 9 was prepared in a similar manner as described in Example 13.1, and was characterized by NMR and MS; LC-MS m/z 179 (M+1); ¹H NMR (400 MHz, CD₃OD) δ 5.07 (t, J=2.2 Hz, 2H), 4.92 (t, J=2.2 Hz, 2H).

### Example 13.10

### Preparation of 5-Ethyl-3-(1H-tetrazol-5-yl)-2,4,5,6-tetrahydro-cyclopentapyrazole (Compound 10)

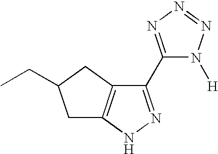

Compound 10 was prepared in a similar manner as described in Example 13.1, and was characterized by NMR and MS; ¹H NMR (MeOD, 400 MHz): δ 3.07 (1H, dd, J=14.8, 7.6 Hz), 2.94-2.82 (2H, m), 2.51 (1H, dd, J=15.2, 6.8 Hz) 2.41 (1H, dd, J=13.6, 5.6 Hz), 1.6 (2H, m), 1.02 (3H, t, J=7.2 Hz). HPLC/MS: Discovery® C18 column (5μ, 50×2.1 mm), 5% v/v CH₃CN (containing 1% v/v TFA) in H₂O (containing 1% v/v TFA) gradient to 99% v/v CH₃CN in H₂O, 0.75 mL/min, tᵣ=1.42 min, ESI⁺=205.2 (M+H).

### Example 13.11

### Preparation of 5-(5-Isopropyl-1H-pyrazol-3-yl)-1H-tetrazole (Compound 11)

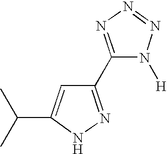

Compound 11 was prepared in a similar manner as described herein or by a method know in the art.

Applicants reserve the right to exclude any one or more compounds from any of the embodiments of the invention. Applicants also reserve the right to exclude, for example, any formulation or amount of niacin, a niacin analog or niacin receptor agonist, any niacin receptor partial agonist, or any combination therapy.

Throughout this application, various publications, patents and published patent applications are cited. The disclosures of these publications, patents and published patent applications referenced in this application are hereby incorporated by reference in their entirety into the present disclosure. Citation herein by Applicant of a publication, patent, or published patent application is not an admission by Applicant of said publication, patent, or published patent application as prior art.

Modifications and extension of the disclosed inventions that are within the purview of the skilled artisan are encompassed within the above disclosure and the claims that follow.

What is claimed is:

1. A method of reducing flushing induced by niacin or a niacin analog in a subject, comprising administering to said subject an effective flush reducing amount of a niacin receptor partial agonist.

2. The method of claim 1, wherein said flushing is induced by niacin.

3. The method of claim 1, wherein said flushing is induced by a niacin analog.

4. The method of claim 1, wherein said niacin analog is a structural analog of niacin.

5. The method of claim 1, wherein said niacin analog is a functional analog of niacin.

6. The method of claim 1, wherein said niacin receptor partial agonist comprises Formula (I): 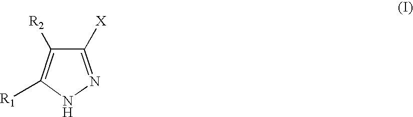
or a pharmaceutically acceptable salt thereof,
wherein:
X is a carboxyl or a tetrazol-5-yl group;
R₁ is iso-propyl, 3-fluoro-benzyl, 3-chloro-benzyl, or 3-bromo-benzyl; and
R₂ is H;
or
R₁ and R₂ together with the two pyrazole ring carbons to which they are bonded form a 5-membered carbocyclic ring optionally substituted with ethyl or a 5-membered heterocyclic ring optionally substituted with methyl.

7. The niacin receptor partial agonist of claim 6, wherein said niacin receptor partial agonist comprises a compound selected from the group consisting of:
3-(1H-Tetrazol-5-yl)-1,4,5,6-tetrahydro-cyclopentapyrazole;
5-(3-Fluoro-benzyl)-1H-pyrazole-3-carboxylic acid;
5-(3-Chloro-benzyl)-1H-pyrazole-3-carboxylic acid;
5-(3-Bromo-benzyl)-1H-pyrazole-3-carboxylic acid;
6-Methyl-3-(1H-tetrazol-5-yl)-4,6-dihydro-1H-furo[3,4-c]pyrazole;
3-(1H-Tetrazol-5-yl)-4,6-dihydro-1H-thieno[3,4-c]pyrazole;
3-(1H-Tetrazol-5-yl)-1,4-dihydro-cyclopentapyrazole;
3-(1H-Tetrazol-5-yl)-1,6-dihydro-cyclopentapyrazole;
3-(1H-Tetrazol-5-yl)-2,6-dihydro-4H-furo[3,4-c]pyrazole;
5-Ethyl-3-(1H-tetrazol-5-yl)-2,4,5,6-tetrahydro-cyclopentapyrazole; and
5-(5-Isopropyl-1H-pyrazol-3-yl)-1H-tetrazole;
or a pharmaceutically acceptable salt thereof.

8. A method of reducing flushing induced by niacin or a niacin analog in a subject, comprising administering to said subject an effective flush reducing amount of a niacin receptor partial agonist and an effective lipid altering amount of niacin or a niacin analog.

9. The method of claim 8, wherein said flushing is induced by niacin.

10. The method of claim 8, wherein said flushing is induced by a niacin analog.

11. The method of claim 8, wherein said niacin analog is a structural analog of niacin.

12. The method of claim 8, wherein said niacin analog is a functional analog of niacin.

13. The method of claim 8, wherein said lipid altering amount of niacin or a niacin analog is at least 500 mg per day.

14. The method of claim 8, wherein said niacin receptor partial agonist comprises Formula (I): 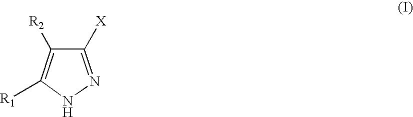
or a pharmaceutically acceptable salt thereof,
wherein:
X is a carboxyl or a tetrazol-5-yl group;
R₁ is iso-propyl, 3-fluoro-benzyl, 3-chloro-benzyl, or 3-bromo-benzyl; and
R₂ is H;
or
R₁ and R₂ together with the two pyrazole ring carbons to which they are bonded form a 5-membered carbocyclic ring optionally substituted with ethyl or a 5-membered heterocyclic ring optionally substituted with methyl.

15. The niacin receptor partial agonist of claim 14, wherein said niacin receptor partial agonist comprises a compound selected from the group consisting of:
3-(1H-Tetrazol-5-yl)-1,4,5,6-tetrahydro-cyclopentapyrazole;
5-(3-Fluoro-benzyl)-1H-pyrazole-3-carboxylic acid;
5-(3-Chloro-benzyl)-1H-pyrazole-3-carboxylic acid;
5-(3-Bromo-benzyl)-1H-pyrazole-3-carboxylic acid;
6-Methyl-3-(1H-tetrazol-5-yl)-4,6-dihydro-1H-furo[3,4-c]pyrazole;
3-(1H-Tetrazol-5-yl)-4,6-dihydro-1H-thieno[3,4-c]pyrazole;
3-(1H-Tetrazol-5-yl)-1,4-dihydro-cyclopentapyrazole;
3-(1H-Tetrazol-5-yl)-1,6-dihydro-cyclopentapyrazole;
3-(1H-Tetrazol-5-yl)-2,6-dihydro-4H-furo[3,4-c]pyrazole;
5-Ethyl-3-(1H-tetrazol-5-yl)-2,4,5,6-tetrahydro-cyclopentapyrazole; and
5-(5-Isopropyl-1H-pyrazol-3-yl)-1H-tetrazole;
or a pharmaceutically acceptable salt thereof.

16. A method for preventing or treating a lipid-associated disorder in a subject, comprising administering to said subject an effective flush reducing amount of a niacin receptor partial agonist and an effective lipid altering amount of niacin or a niacin analog.

17. The method of claim 16, wherein said flushing is induced by niacin.

18. The method of claim 16, wherein said flushing is induced by a niacin analog.

19. The method of claim 16, wherein said niacin analog is a structural analog of niacin.

20. The method of claim 16, wherein said niacin analog is a functional analog of niacin.

21. The method of claim 16, wherein said lipid altering amount of niacin or a niacin analog is at least 500 mg per day.

22. The method of claim 16, wherein said niacin receptor partial agonist comprises Formula (I): 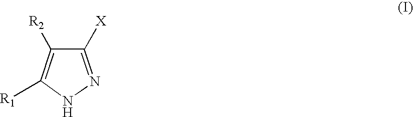
or a pharmaceutically acceptable salt thereof,
wherein:
X is a carboxyl or a tetrazol-5-yl group;
R₁ is iso-propyl, 3-fluoro-benzyl, 3-chloro-benzyl, or 3-bromo-benzyl; and
R₂ is H;
or
R₁ and R₂ together with the two pyrazole ring carbons to which they are bonded form a 5-membered carbocyclic ring optionally substituted with ethyl or a 5-membered heterocyclic ring optionally substituted with methyl.

23. The niacin receptor partial agonist of claim 22, wherein said niacin receptor partial agonist comprises a compound selected from the group consisting of:
3-(1H-Tetrazol-5-yl)-1,4,5,6-tetrahydro-cyclopentapyrazole;
5-(3-Fluoro-benzyl)-1H-pyrazole-3-carboxylic acid;
5-(3-Chloro-benzyl)-1H-pyrazole-3-carboxylic acid;
5-(3-Bromo-benzyl)-1H-pyrazole-3-carboxylic acid;
6-Methyl-3-(1H-tetrazol-5-yl)-4,6-dihydro-1H-furo[3,4-c]pyrazole;
3-(1H-Tetrazol-5-yl)-4,6-dihydro-1H-thieno[3,4-c]pyrazole;
3-(1H-Tetrazol-5-yl)-1,4-dihydro-cyclopentapyrazole;
3-(1H-Tetrazol-5-yl)-1,6-dihydro-cyclopentapyrazole;
3-(1H-Tetrazol-5-yl)-2,6-dihydro-4H-furo[3,4-c]pyrazole;
5-Ethyl-3-(1H-tetrazol-5-yl)-2,4,5,6-tetrahydro-cyclopentapyrazole; and
5-(5-Isopropyl-1H-pyrazol-3-yl)-1H-tetrazole;
or a pharmaceutically acceptable salt thereof.

24. The method of claim 16, further comprising administering to said subject at least one agent selected from the group consisting of α-glucosidase inhibitor, aldose reductase inhibitor, biguanide, HMG-CoA reductase inhibitor, squalene synthesis inhibitor, fibrate, LDL catabolism enhancer, angiotensin converting enzyme inhibitor, insulin secretion enhancer and thiazolidinedione.

25. A composition for administration of an effective lipid altering amount of niacin or a niacin analog having reduced capacity to provoke a flushing reaction in a subject, comprising
(a) an effective lipid altering amount of niacin or a niacin analog, and
(b) an effective flush reducing amount of a niacin receptor partial agonist.

26. A kit for preventing or treating a lipid-associated disorder comprising at least one dosage unit of a niacin receptor partial agonist and at least one dosage unit of niacin or a niacin analog, wherein said niacin receptor partial agonist is present in an amount effective to reduce flushing induced by niacin, or a niacin analog in said subject and wherein said niacin or niacin analog is present in a lipid altering amount.

27. A kit for preventing or treating a lipid-associated disorder comprising at least one dosage unit of a niacin receptor partial agonist and at least one separate dosage unit of niacin or a niacin analog, wherein said niacin receptor partial agonist is present in an amount effective to reduce flushing induced by niacin or a niacin analog in said subject and wherein said niacin or niacin analog is present in a lipid altering amount.

28. A kit for preventing or treating a lipid-associated disorder comprising at least one pre-dosage unit of a niacin receptor partial agonist and at least one separate dosage unit of niacin or a niacin analog, wherein said niacin receptor partial agonist is present in an amount effective to reduce flushing induced by niacin or a niacin analog in said subject and wherein said niacin or niacin analog is present in a lipid altering amount.


SEQUENCE LISTING (7 sequences)

SEQ ID NO: 1 - DNA, 1092 bp, Homo sapiens
atgaatcggcaccatctgcaggatcactttctggaaatagacaagaagaactgctgtgtgttccgagatgacttcattgtcaaggtgttgccgccggtgttggggctggagtttatcttcgggcttctgggcaatggccttgccctgtggattttctgtttccacctcaagtcctggaaatccagccggattttcctgttcaacctggcagtggctgactttctactgatcatctgcctgcccttcctgatggacaactatgtgaggcgttgggactggaagtttggggacatcccttgccggctgatgctcttcatgttggctatgaaccgccagggcagcatcatcttcctcacggtggtggcggtagacaggtatttccgggtggtccatccccaccacgccctgaacaagatctccaatcggacagcagccatcatctcttgccttctgtggggcatcactattggcctgacagtccacctcctgaagaagaagatgccgatccagaatggcggtgcaaatttgtgcagcagcttcagcatctgccataccttccagtggcacgaagccatgttcctcctggagttcttcctgcccctgggcatcatcctgttctgctcagccagaattatctggagcctgcggcagagacaaatggaccggcatgccaagatcaagagagccatcaccttcatcatggtggtggccatcgtctttgtcatctgcttccttcccagcgtggttgtgcggatccgcatcttctggctcctgcacacttcgggcacgcagaattgtgaagtgtaccgctcggtggacctggcgttctttatcactctcagcttcacctacatgaacagcatgctggaccccgtggtgtactacttctccagcccatcctttcccaacttcttctccactttgatcaaccgctgcctccagaggaagatgacaggtgagccagataataaccgcagcacgagcgtcgagctcacaggggaccccaacaaaaccagaggcgctccagaggcgttaatggccaactccggtgagccatggagcccctcttatctgggcccaacctctccttaa

SEQ ID NO: 2 - protein, 363 aa, Homo sapiens [sequence omitted]

SEQ ID NO: 3 - protein, 6 aa, Artificial sequence
  Synthetically generated peptide
TLESIM

SEQ ID NO: 4 - protein, 5 aa, Artificial sequence
  Synthetically generated peptide
EYNLV

SEQ ID NO: 5 - protein, 5 aa, Artificial sequence
  Synthetically generated peptide
DCGLF

SEQ ID NO: 6 - DNA, 36 bp, Artificial sequence
  primer
gatcaagcttccatggcgtgctgcctgagcgaggag

SEQ ID NO: 7 - DNA, 53 bp, Artificial sequence
  primer
gatcggatccttagaacaggccgcagtccttcaggttcagctgcaggatggtg